(19) United States
(12) Patent Application Publication
(10) Pub. No.: US 2024/0352446 A1
(43) Pub. Date: Oct. 24, 2024

(54) DETERMINING LEARNING PHENOTYPE AND GENOTYPE VIA MUTATIONAL RECORDING AND SEQUENCING

(71) Applicant: Massachusetts Institute of Technology, Cambridge, MA (US)

(72) Inventors: Kevin Michael Esvelt, Newton, MA (US); Boqiang Tu, Somerville, MA (US)

(73) Assignee: Massachusetts Institute of Technology, Cambridge, MA (US)

(21) Appl. No.: 18/685,590

(22) PCT Filed: Aug. 23, 2022
(86) PCT No.: PCT/US2022/075325

§ 371 Date: Feb. 22, 2024

Related U.S. Application Data
(60) Provisional application No. 63/235,907, filed on Aug. 23, 2021.

(51) Int. Cl. C12N 15/10

(52) U.S. Cl. CPC C12N 15/1058; C12N 15/1068

(57) ABSTRACT

The invention relates, in part, to methods and systems with which to combine methods of determining an activity of a gene of interest and the gene's sequence, thereby allowing the genotype and associated phenotype to be determined in a single sequencing step.

### RELATED APPLICATIONS

This application claims the benefit under 35 U.S.C. § 119(e) of U.S. Provisional application Ser. No. 63/235,907 filed Aug. 23, 2021, the disclosure of which is incorporated by reference herein in its entirety.

### FIELD OF THE INVENTION

The invention relates, in part, to methods of assessing gene activity and sequencing the gene to obtain phenotype and genotype information.

### BACKGROUND OF THE INVENTION

Current methods of measuring the molecular activity of a gene-encoded biomolecule typically links the activity to production of an optically active molecule such as luciferase or green fluorescent protein, then measures the resulting signal in a plate reader or flow cytometer to determine the phenotype. Sequencing to determine which nucleic acid sequence is present must be performed independently to ascertain the genotype.

### SUMMARY OF THE INVENTION

According to an aspect of the invention, a method of determining a sequence and activity of a preselected gene of interest is provided, the method including: (a) preparing a composition that includes a preselected gene of interest, a canvas polynucleotide sequence, and a polynucleotide sequence encoding a mutagenic protein, wherein the preselected gene of interest is contiguous with the canvas polynucleotide sequence and when expressed, activity of the encoded mutagenic protein accumulates a detectable mutation in the canvas polynucleotide sequence at a rate proportional to a molecular activity of the expression product of the preselected gene of interest; (b) positioning the prepared composition in a transcription/translation-suitable (TTS) environment; (c) expressing the preselected gene of interest and the encoded mutagenic protein in the TTS environment; (d) extracting DNA from the TTS environment at a time after the expressing; (e) sequencing the preselected gene of interest and the canvas polynucleotide sequence in the extracted DNA; and (t) counting a number of the detectable mutation in the canvas polynucleotide sequence, wherein the counted number of the detectable mutation is proportional to the activity of the sequenced preselected gene of interest, and the sequencing and counting determines the sequence and activity of the preselected gene of interest. In certain embodiments, the T'S environment is a transcription/translation (TT) reaction vessel. In some embodiments, the TTS environment is an in vitro cell. In some embodiments, the in vitro cell is a cultured cell. In certain embodiments, the cell is a bacterial cell or an archaeal cell. In some embodiments, the cell is a eukaryotic cell. In some embodiments, the cell is a mammalian cell, an insect cell, a plant cell, or a fungal cell. In certain embodiments, the cell is a non-human mammalian cell. In certain embodiments, the mutagenic protein includes an enzyme. In some embodiments, the activity of the mutagenic protein randomly introduces the detectable mutation in the canvas polynucleotide sequence. In some embodiments, the activity of the mutagenic protein introduces the detectable mutation at one or more specific sites in the canvas polynucleotide sequence. In some embodiments, the activity of the mutagenic protein introduces the detectable mutation at one or more specific sites contiguous with the polynucleotide encoding the preselected gene of interest. In certain embodiments, the detectable mutation is introduced 5′ of the polynucleotide encoding the preselected gene of interest. In some embodiments, the detectable mutation is introduced 3′ of the polynucleotide encoding the preselected gene of interest. In certain embodiments, the mutagenic protein introduces the detectable mutation within the polynucleotide sequence encoding the preselected gene of interest, wherein the introduction does not disrupt a genotypic information of the preselected gene of interest. In some embodiments, the detectable mutation is introduced into one or more of: (a) an intron in the polynucleotide encoding the preselected gene of interest, and (b) one or more synonymous bases of the polynucleotide encoding the preselected gene of interest. In some embodiments, the mutagenic protein introduces an epigenetic change in the polynucleotide encoding the preselected gene of interest, wherein the epigenetic change is detectable by sequencing, optionally by nanopore sequencing. In some embodiments, the enzyme is a deaminase, a terminal transferase, a nuclease, a recombinase, or a methylase. In certain embodiments, the enzyme is a base editor attached to a DNA-binding protein that binds to one or more sites adjacent to or within the polynucleotide encoding the preselected gene of interest. In some embodiments, the enzyme is a base editor and the canvas polynucleotide sequence includes one or more guide RNA target sites for the base editor. In some embodiments, the method also includes, expressing one or a plurality of guide RNAs capable of directing the base editor to one or more target polynucleotide sequences. In certain embodiments, the expressed one or the plurality of guide RNAs are expressed by at least one guide RNA-expressing array. In some embodiments, the enzyme is a CRISPR base editor, a CRISPR nuclease, a CRISPR prime editor, or a CRISPR spacer acquisition enzyme. In some embodiments, the enzyme is a mutagenic polymerase that moves along the polynucleotide encoding the preselected gene of interest or a sequence adjacent to the 5′ or 3′ end of the polynucleotide sequence encoding the preselected gene of interest. In some embodiments, the enzyme is a retron. In certain embodiments, the method also includes multiplexing the mutagenic protein and mutagenizing multiple nucleic acid sequences contiguous with the polynucleotide sequence of the preselected gene of interest. In some embodiments, the method also includes increasing a length of time before extracting the DNA, wherein the increased length of time increases the accumulation of mutations in the canvas polynucleotide sequence. In certain embodiments, the preselected gene of interest is a gene encoding a: oxidoreductase, transferase, hydrolase, lyase, isomerase, ligase (all six major enzyme classes), DNA-binding protein, RNA-binding protein, protein-binding protein, lipid-binding protein. In some embodiments, the preselected gene of interest is a gene encoding a recombinase, an integrase, a protease, a polymerase, a reverse transcriptase, a nuclease, a nickase, a tRNA, aminoacyl tRNA synthetase, or a ribosome. In some embodiments, the canvas polynucleotide sequence includes one or more predetermined polynucleotide sequences. In certain embodiments, the predetermined polynucleotide sequence includes a repeated nucleic acid sequence. In some embodiments, the repeated nucleic acid sequence includes 2, 3, 4, 5, 6, 7, 8, 9, 10 or more repeats of a preselected nucleic acid sequence. In some embodiments, the repeated nucleic acid sequence includes a TetO array. In some embodiments, the predetermined polynucleotide sequence includes one or more of a gI, gIV, and gVI sequence of an M13 bacteriophage. In certain embodiments, the method also includes (a) extracting DNA from the TIS environment two or more times after the expressing: (b) counting a number of the detectable mutation in the canvas polynucleotide sequence in the two or more DNA extractions; and (c) comparing the sequence of the preselected gene of interest and the number of counted detectable mutations in at least two of the two or more DNA extractions. In some embodiments, the two or more DNA extractions are separated by one or more of: at least 1 min., 5 min., 10 min., 20 min., 30 min., 40 min., 50 min., 60 min., 120 min., 180 min., 240 min., 300 min., 360 min., 420 min., 480 min., 540 min., 10 hr., 12 hr., 15 hr. 20 hr. 24 hr., 36 hr., 48 hr., 60 hr., 72 hr., 96 hr., 192 hr. 384 hr., and 800 hr. In some embodiments, a length of time between any two of the two or more DNA extractions is independently selected. In certain embodiments, a means for one or more of the extracting, sequencing, and counting methods includes a microfluidics method. In some embodiments, the composition also includes a polynucleotide sequence encoding a detectable protein; the detectable protein is expressed in the TTS environment; and the level of detectable protein expressed is relative to the level of the expression product of the preselected gene of interest. In some embodiments, the detectable protein is a fluorescent or luminescent protein. In some embodiments, the TTS reaction vessel includes a plurality of the compositions each including an independently selected preselected gene sequence of interest. In certain embodiments, the method also includes determining a pattern of the detectable mutation in the canvas polynucleotide sequence, wherein the determining occurs following the sequencing step.

According to another aspect of the invention, a method of determining sequences and activities of a plurality of independently preselected genes of interest are provided, the method including: (a) preparing a plurality of compositions, each including an independently preselected gene of interest adjacent to a canvas polynucleotide sequence and a polynucleotide sequence encoding a mutagenic protein, wherein, when expressed, activity of the encoded mutagenic protein in each composition accumulates the detectable mutation in the canvas polynucleotide sequence in the composition at a rate proportional to the molecular activity of the expression product of the independently selected preselected gene of interest in the composition; (b) positioning the plurality of the prepared compositions in a transcription/translation-suitable (TTS) environment; (c) expressing the preselected genes of interest and the encoded mutagenic proteins in the TTS environment; (d) extracting DNA from the TTS environment at a time after the expressing; (e) sequencing the preselected genes of interest and the canvas polynucleotide sequences in the extracted DNA; and (f) counting a number of the detectable mutation in the canvas polynucleotide sequences, wherein the counted numbers of the detectable mutations are proportional to the activity of the sequenced preselected gene of interest in each of the plurality of compositions, and the sequencing and counting determines the sequences and activities of the independently preselected genes of interest. In some embodiments, the method also includes physically separating the compositions before expressing the preselected genes of interest and the encoded mutagenic proteins. In some embodiments, the physically separating occurs before extracting DNA from the TTS environment. In certain embodiments, a means of the sequencing includes one or more of: a high-throughput sequencing method, a Sanger sequencing method, and a barcoded high-throughput sequencing method. In some embodiments, the extracted DNA is pooled together and sequenced. In some embodiments, a means of the sequencing the pooled DNA includes a high-throughput sequencing method. In some embodiments, a means for the sequencing includes a nanopore, a PacBio, or an Illumina sequencing method. In some embodiments, the method also includes physically isolating each encoding polynucleotide sequence or a plurality of identical encoding polynucleotide sequences within a cell. In certain embodiments, the cell is a bacterial or archaeal cell. In some embodiments, the cell is a eukaryotic cell. In some embodiments, the cell is a mammalian cell. In certain embodiments, the cell is a non-human mammalian cell. In some embodiments, the method also includes physically isolating each encoding polynucleotide sequence or a plurality of identical encoding polynucleotide sequences within an emulsion. In certain embodiments, the method also includes (a) encoding the polynucleotide sequence(s) on phages or viruses; (b) infecting a reporter cell or plurality of reporter cells with the phages or viruses, wherein the infection includes approximately one virus per reporter cell, wherein the reporter cell or plurality of cells each encode a recording machinery targeting a contiguous sequence in the phage or virus genome. In some embodiments, the method also includes subjecting the polynucleotide sequence(s) to one or more of screening, selection, and directed evolution prior to the encoding of the polynucleotide sequence(s) on the phages or viruses. In some embodiments, the method also includes subjecting the phages or viruses encoding the polynucleotide sequence to one or more of screening, selection, and directed evolution prior to infection of the reporter cell or plurality of reporter cells. In certain embodiments, the method also includes detecting an activity of the reporter cell or plurality of reporter cells, wherein the detected activity of the reporter cell or each of the plurality of reporter cells informs an activity of all members of the evolving population. In certain embodiments, the method also includes detecting an activity of each reporter cell, wherein the detected activity of the reporter cell informs an activity of an individual member of the evolving population. In some embodiments, the method also includes generating or identifying the plurality of independently preselected genes of interest. In some embodiments, the plurality of independently preselected genes of interest encode a corresponding plurality of proteins, each capable of an individual activity level. In some embodiments, the method also includes (i) physically isolating the expressed proteins from one another at a time subsequent to the step prior to the expressing step; and (ii) predicting activities of one or more proteins encoded by genes outside the plurality of independently preselected genes of interest based at least in part on the sequences and activities of the plurality of independently preselected genes of interest determined in the sequencing and counting steps. In certain embodiments, a means for the predicting includes a machine learning method. In certain embodiments, the sequences and activities determined in the sequencing and counting steps include a training set for the machine learning method. In some embodiments, the method also includes applying the machine learning method and generating novel variants of one or more of the independently selected genes of interest. In some embodiments, the method also includes determining a pattern of the detectable mutation in one or more of the canvas polynucleotide sequences, wherein the determining occurs following the sequencing step.

According to another aspect of the invention, a composition is provided, that includes: (i) a preselected gene of interest contiguous to a canvas polynucleotide sequence and (ii) a polynucleotide sequence encoding a mutagenic protein, wherein, when expressed, activity of the encoded mutagenic protein accumulates a detectable mutation in the canvas polynucleotide sequence at a rate proportional to a molecular activity of the expression product of the preselected gene of interest. In certain embodiments, the mutagenic protein is or includes an enzyme. In some embodiments, the mutagenic protein is capable of randomly introducing the detectable mutation in the canvas polynucleotide sequence. In some embodiments, the mutagenic protein is capable of introducing the detectable mutation in one or more specific sites adjacent to the polynucleotide encoding the preselected gene of interest. In some embodiments, the mutagenic protein is capable of introducing the detectable mutation 5′ of the polynucleotide encoding the preselected gene of interest. In certain embodiments, the mutagenic protein is capable of introducing the detectable mutation 3′ of the polynucleotide encoding the preselected gene of interest. In certain embodiments, the mutagenic protein is capable of introducing the detectable mutation within the polynucleotide sequence encoding the preselected gene of interest, and the introduction does not disrupt a genotypic information of the preselected gene of interest. In some embodiments, the mutagenic protein is capable of introducing the detectable mutation into one or more of: (a) an intron in the polynucleotide encoding the preselected gene of interest and (b) one or more synonymous bases of the polynucleotide encoding the preselected gene of interest. In some embodiments, the mutagenic protein is capable of introducing an epigenetic change in the polynucleotide encoding the preselected gene of interest In certain embodiments, the epigenetic change is detectable by sequencing, optionally nanopore sequencing. In some embodiments, the enzyme is a deaminase, a terminal transferase, a nuclease, a recombinase, or a methylase. In some embodiments, the enzyme is a base editor attached to a DNA-binding protein that binds to one or more sites adjacent to or within the polynucleotide encoding the preselected gene of interest. In some embodiments, the composition also includes one or a plurality of guide RNAs capable of targeting the base editor. In certain embodiments, the enzyme is a CRISPR base editor, CRISPR nuclease, or a CRISPR prime editor. In some embodiments, the enzyme is a mutagenic polymerase capable of moving along the polynucleotide encoding the preselected gene of interest or a sequence adjacent to the 5′ or 3′ end of the polynucleotide sequence encoding the preselected gene of interest. In some embodiments, the enzyme is a retron. In certain embodiments, the preselected gene of interest is a gene encoding a wherein the preselected gene of interest is a gene encoding a: oxidoreductase, transferase, hydrolase, lyase, isomerase, ligase (all six major enzyme classes), DNA-binding protein, RNA-binding protein, protein-binding protein, lipid-binding protein. If we need be more specific, recombinase, integrase, protease, polymerase, reverse transcriptase, nuclease, nickase, tRNA, aminoacyl tRNA synthetase, or ribosome. In some embodiments, the canvas polynucleotide sequence includes one or more predetermined polynucleotide sequences. In some embodiments, the predetermined polynucleotide sequence includes a repeated nucleic acid sequence. In certain embodiments, the repeated nucleic acid sequence includes 2, 3, 4, 5, 6, 7, 8, 9, 10 or more repeats of a preselected nucleic acid sequence. In some embodiments, the repeated nucleic acid sequence includes a TetO array. In some embodiments, the predetermined polynucleotide sequence includes one or more of a gI, gIV and gVI sequence of an M13 bacteriophage. In certain embodiments, the composition also includes a polynucleotide sequence encoding a detectable protein. In certain embodiments, the detectable protein is a fluorescent or luminescent protein.

According to another aspect of the in invention, a method of determining a sequence and activity of a preselected genes of interest is provided, the method including: (a) preparing a composition of any embodiment of an aforementioned aspect of the invention, (b) positioning the prepared composition in a transcription/translation-suitable (TTS) environment; expressing the preselected gene of interest and the encoded mutagenic protein in the TTS environment; (c) extracting DNA from the TTS environment at a time after the expressing; (d) sequencing the preselected gene of interest and the canvas polynucleotide sequence in the extracted DNA; and (e) assessing the detectable mutation in the canvas polynucleotide sequence, wherein the assessment of the detectable mutation correlates with the activity of the sequenced preselected gene of interest, and the sequencing and assessing determines the sequence and activity of the preselected gene of interest. In some embodiments, the assessing includes counting a number of the detectable mutation in the canvas polynucleotide and wherein the counted number of the detectable mutations are proportional to the activity of the sequenced preselected gene of interest in each of the plurality of compositions. In some embodiments, the assessing includes determining a pattern of the detectable mutations in the canvas polynucleotide.

According to another aspect of the invention, a method of determining sequences and activities of a plurality of independently preselected genes of interest is provided, the method including (a) preparing a plurality of compositions of any embodiment of an aforementioned aspect of the invention, each composition including an independently preselected gene of interest adjacent to a canvas polynucleotide sequence and a polynucleotide sequence encoding a mutagenic protein, wherein, when expressed, activity of the encoded mutagenic protein in each composition accumulates the detectable mutation in the canvas polynucleotide sequence in the composition at a rate proportional to the molecular activity of the expression product of the independently selected preselected gene of interest in the composition; (b) positioning the plurality of the prepared compositions in a transcription/translation-suitable (TTS) environment; (c) expressing the preselected genes of interest and the encoded mutagenic proteins in the TTS environment; (d) extracting DNA from the TTS environment at a time after the expressing; (e) sequencing the preselected genes of interest and the canvas polynucleotide sequences in the extracted DNA; and (f) assessing the detectable mutation in the canvas polynucleotide sequences, wherein the assessment of the detectable mutations correlates with the activity of the activity of the sequenced preselected gene of interest in each of the plurality of compositions, and the sequencing and assessing determines the sequences and activities of the independently preselected genes of interest. In some embodiments, the assessing includes counting a number of the detectable mutation in the canvas polynucleotide, and wherein the counted number of the detectable mutations are proportional to the activity of the sequenced preselected gene of interest in each of the plurality of compositions. In certain embodiments, the assessing includes determining a pattern of the detectable mutations in the canvas polynucleotide. In some embodiments, the assessing comprises counting numbers of the detectable mutation in the canvas polynucleotide in samples collected during continuous growth at different time points, and wherein the logarithmically transformed maximum rate of mutation accumulation is proportional to the activity of the sequenced preselected gene of interest in each of the plurality of compositions.

### BRIEF DESCRIPTION OF THE DRAWINGS

FIG. 1 presents a flowchart illustrating a typical workflow for a molecular activity assay of physically separated nucleic acids.

FIG. 2 presents graphs illustrating cytidine to thymidine mutation rates within targeted sequences in proportion to the magnitude of molecular activity of nucleic acid measured. The nucleic acid encoding T7 RNA polymerase in these experiments was expressed under the control of an IPTG (isopropyl β-D-1-thiogalactopyranoside)-inducible promoter. In each group of four bars at each IPTG dosage level, the left-most bar shows the C to T mutation rate at 0.0 hours, the center-left bar shows the C to T mutation rate at 6.0 hours, the center-right bar shows C to T mutation rate at 24.0 hours, and the rightmost bar shows the C to T mutation rate at 40.0 hours.

FIG. 3 is a schematic diagram of a base editor map used in certain embodiments of the invention.

FIG. 4 provides schematic diagram of a guide array used in certain embodiments of the invention.

FIG. 5 provides bar graphs showing rate of targeted C to T transitions in phage variants propagated in reporter cells. Phage variants encoding T7 polymerase with various levels of activity on T7 promoter were collected from a continuous evolution experiments. Reporter cells were infected with phage variants, and samples were collected after 12 hrs of incubation. Canvas fragments were then directly amplified and barcoded for nanopore sequencing and numbers of C to T mutations were counted for each of the 10 regions targeted by the base editor using a guide array.

FIG. 6A-B provides a schematic of design of an example of a plasmid construct and a schematic diagram of a general workflow of an embodiment of a system of the invention. Design of plasmid constructs and general workflow. FIG. 6A shows annotation of a typical “recorder plasmid (RP)” construct used for system validation. Base editor and GFP are transcribed in opposite directions and insulated with a strong terminator upstream in each direction. Identical promoters and RBSs used for both coding sequences allow calibration of base editor activity in the form of mutations recorded against fluorescence signal corresponding to such activity. FIG. 6B shows general workflow of a direct high-throughput activity recording and measurement assay (DHARMA).

FIG. 7 provides graphs showing Representative mutation profiles of the canvas region over time. Four conditions were chosen to test the effects of base editor promoter strength, sgRNA promoter strength, and their interaction on base editing activity observed as accumulation of mutations in the canvas region. Condition A: weak base editor promoter/weak sgRNA promoter, condition B: weak base editor promoter/strong sgRNA promoter, condition C: strong base editor promoter/weak sgRNA promoter, and condition D: strong base editor promoter/strong sgRNA promoter.

FIG. 8A-B provides schematic plasmid maps. The maps of plasmids are examples of those used in cloning and system validation. FIG. 8A shows a vector plasmid for library cloning. PaqCI sites flanking the lacZα cassette allow scarless Golden Gate cloning of library fragments directly upstream of base editor and GFP. Blue-white screening can be used to estimate cloning efficiency and minimize background. FIG. 8B shows sgRNA expression plasmid, apFAB36, a strong constitutive promoter drives the expression of sgRNA targeting the repeats in canvas.

FIG. 9A-C provides graphs of result validation studies of DHARMA performance across 24 promoters with a wide range of activities. FIG. 9A is a graph, which, as in Table 2 shows data from insulated promoters fitted to generalized logistic functions. Mutation rate was set to 500 at t=0 to account for baseline mutations and sequencing errors. FIG. 9B is a graph showing the log-transformed total number of C to T mutations in the canvas region plotted against the corresponding log-transformed GFP fluorescence intensity for each insulated promoter at different sampling time. FIG. 9C is a graph of Vmax calculated from fitted curves in FIG. 7B plotted against log-transformed fluorescence intensity. Error bars represent standard deviation.

FIG. 10 provides graph of results of validation of DHARMA performance across 24 promoters with a wide range of activities. Data were collected as in FIG. 9. The graph shows the log-transformed total number of C to T mutations in the canvas region at 8 h after electroporation plotted against the corresponding log-transformed GFP fluorescence intensity for each library member. Error bar represents standard deviation. Inset; plot of insulated promoters.

FIG. 11A-B provides schematic plasmid maps used in T7 polymerase library screening. FIG. 11A shows a vector plasmid for library cloning. SapI sites allow scarless Golden Gate cloning of library fragments directly into the T7 RNA polymerase coding sequence. FIG. 11B shows an sgRNA expression plasmid, apFAB36, a strong constitutive promoter drives the expression of sgRNA targeting the repeats in canvas.

FIG. 12A-C provides graphs and a diagram of results of screening of a library of T7 RNA polymerase variants on T3 promoter with saturation mutagenesis at three AA residues. FIG. 12A shows distribution of number of C to T mutations in canvas region normalized by number of reads obtained for each individual variant. Each data point represents the polymerase activity on T3 promoter of an individual member in the library. FIG. 12B is an amino acid residue logo plot at positions 748, 756 and 758. Height of individual character is proportional to frequency in top 1% of active variants. FIG. 12C is a graph showing normalized C-to-T mutations plotted again log-transformed luminescence adjusted by growth. Each point represents the polymerase activity on T3 promoter of an individual member in the library. Variants were either randomly picked from the whole library or from the most active variants identified in the molecular recording-based assay.

### BRIEF DESCRIPTION OF SEQUENCES

SEQ ID NO: 1 is the sequence of an embodiment of a guide RNA array, referred to herein as Array 1:

ttgacagtagatcagagggttgctataatcgacagttccttctggtaactttgttgttttagatcacgaaagtgaaaagttaaaataagcctagccc
gttaccaactggaaacagtgacttaagaccgccggtcttgtccactaccttgcagtaatgcggtggacaggatcggcggttttcttttcttcact
tctcgttggcacgaaaagggcaataagatttacggattactatcttgacactaccgagacagtgacatataataggaccacaccctgaacaa
agtcagagttgtagagctagcaatagcaggttacaataaggctcgtccgttataaacatgaaaatgtgactaaaaaggccgctctgcggcctt
ttttctttttgcggataaagttgatacccttacctgagttcttctgaaaataacggactttgacacgatgcttgctgctacctataataacatatacc
gaccgtgtgataaatagatttcgagctaggcatagcaagtgaaattaaggctggtccattaacaccttgaaaaagggaacaataaggcctcc
ctttagggggggccttttttattgatgaaaagcaatccctcgtgaagtaactcaatagtgttctctggtatcgtattgacaactgctcagcgaaat
actataatgactacacatgctcgtaaattaggatttttcagatttggaaacaaaacgttgaaaaaaggcaagtccgttatgaacgcgaaagcgt
gcgaaaaaacccgcttcggcgggtttttttatagttacaatcagcagtcagaacttttacgaagaatagtggtcgctcaaccttttgacaggtga
acgctcagctcttataatgcctatcgaacctcccgacttgcggggatgtagatgtagaaatacaaggttacattaaggcccgtccgtaatcaa
cttgaagaagtgttccatcgggtccgaattttcggaccttttctccgcagtgaacgacactactatttcttacgagatacttattctggaagcaac
ggtttgacaaagtactactgtattagtataattgtcattataacccaacctaagccgggttttggacctagaaataggaagtcaaaataaggctg
gaccgacatgtaatcgaaagatttagaaaaaagcccgcacctgacagtgcgggctttttttttcgatattcacttccctcacagattcgttcaga
gataaaagcgttggtaacagtttgacatgcgtgatttaacattctataattgcacatacccgttcttggaatgatagttggagagcaagacattg
caagttccaataaggcgtgtccgataaaagcttgagaaagcaaagtaatacaaaacaggcccaggcggcctgttttgtctttttaatgctgga
atacagataaggatagcgtcgttacaatagtcactcgtagaacttttgacataagtcgtattcaaagatataatataggtgcctcgcgttcttaga
atacatctgagagccaaaaatggcaagttcagataaggccagaccgttaccagcttaaataagcgatcctaaagccccgaattttttataaatt
cggggcttttttactaggagattactttacagaagactcacttatttcacggaactggtgctgacaattgacagaccttatctacatggttataatc
tgaattcctacgatgaaaataaaaagcttcagatccagaaatggaaagttgaagtgaggcaggtccggtagcaactcgaaagagtgagaaa
agaggggagcgggaaaccgctccccttttttcgtttttgattgagatttcaggactgtttaccgaaccttacactacgagcgataattgacacg
gatcttcgctgaacgtataatgagaaaccaacatgtaatttaggcagggaatagaaaacaaaagtttaagttattctaaggccagtccggaat
catcctaaaaaggagttattgaacacccgaaagggtgtttttttgttttagcccgtgtcttcttactggtggataataaagcaactgaacaacgat
tt.

SEQ ID NO: 2 is the sequence of an embodiment of a guide RNA, referred to herein as: Array 2:

ttgacagtagatcagagggttgctataatcgacagtataacccaacctaagccgggttttagatcacgaaagtgaaagttaaaataagcctag
cccgttaccaactggaaacagtgacttaagaccgccggtcttgtccactaccttgcagtaatgcggtggacaggatcggcggttttcttttcttc
acttctctgttggcacgaaaagggcaataagatttacggattactatcttgacactaccgagacagtgacatataataggaccttccttctggta
actttgttgttgtagagctagcaatagcaggttacaataaggctcgtccgttataaacatgaaaatgtgactaaaaaggccgctctgcggccttt
tttctttttgcggataaagttgatacccttacctgagttcttctgaaaataacggactttgacacgatgcttgctgctacctataataacatata
cccgttcttggaatgatagatttcgagctaggcatagcaagtgaaattaaggctggtccattaacaccttgaaaaagggaacaataaggcctccc
tttagggggggccttttttattgatgaaaagcaatccctcgtgaagtaactcaatagtgttctctggtatcgtattgacaactgctcagcgaaa
tactataatgactacacaccctgaacaaagtcagattttcagatttggaaacaaaacgttgaaaaaaggcaagtccgttatgaacgcgaaagcgt
gcgaaaaaacccgcttcggcgggtttttttatagttacaatcagcagtcagaacttttacgaagaatagtggtcgctcaaccttttgacaggtga
acgctcagctcttataatgcctattaccgaccgtgtgataaatagatgtagatgtagaaatacaaggttacattaaggcccgtccgtaatcaact
tgaagaagtgttccatcgggtccgaattttcggaccttttctccgcagtgaacgacactactatttcttacgagatacttattctggaagcaacg
gtttgacaaagtactactgtattagtataattgtcatgcctcgcgttcttagaatacgttttggacctagaaataggaagtcaaaataaggctgg
accgacatgtaatcgaaagatttagaaaaaagcccgcacctgacagtgcgggctttttttttcgatattcacttccctcacagattcgttcagag
ataaaagcgttggtaacagtttgacatgcgtgatttaacattctataattgcacacgaacctcccgacttgcggggttggagagcaagacattgc
aagttccaataaggcgtgtccgataaaagcttgagaaagcaaagtaatacaaaacaggcccaggcggcctgttttgtctttttaatgctggaata
cagataaggatagcgtcgttacaatagtcactcgtagaacttttgacataagtcgtattcaaagatataatataggtccaacatgtaatttaggc
agatctgagagccaaaaatggcaagttcagataaggccagaccgttaccagcttaaataagcgatcctaaagccccgaattttttataaattcg
gggcttttttactaggagattactttacagaagactcacttatttcacggaactggtgctgacaattgacagaccttatctacatggttataatc
tgaattcctacgatgaaaataaaaagcttcagatccagaaatggaaagttgaagtgaggcaggtccggtagcaactcgaaagagtgagaaaa
gaggggagcgggaaaccgctccccttttttcgtttttgattgagatttcaggactgtttaccgaaccttacactacgagcgataattgacacgg
atcttcgctgaacgtataatgagaaaacatgctcgtaaattaggatggaatagaaaacaaaagtttaagttattctaaggccagtccggaatca
tcctaaaaaggagttattgaacacccgaaagggtgtttttttgttttagcccgtgtcttcttactggtggataataaagcaactgaacaacgatt
t.

SEQ ID NO: 3 is sequence of an embodiment of a canvas polynucleotide sequence:

gtcgacatgccagttcttttgggtattccgttattattgcgtttcctcggtttccttctggtaactttgttcggctatctgcttacttttcttaaaa
agggcttcggtaagatagctattgctatttcattgtttcttgctcttaattattgggcttaactcaattcttgtgggttatctctctgatattagcg
ctcaattacccctctgactttgttcagggtgttcagttaattctcccgtctaatgcgcttccctgtttttatgttattctctctgtaaaggctgcta
ttttcatttttgacgttaaacaaaaaatcgtttcttatttggattgggataaataatatggctgtttattttgtaactggcaaattaggctctggaa
agacgctcgttagcgttggtaagattcaggataaaattgtagctgggtgcaaaatagcaactaatcttgatttaaggcttcaaaacctcccgcaagt
cgggaggttcgctaaaacgcctcgcgttcttagaataccggataagccttctatatctgatttgcttgctattgggcgcggtaatgattcctacgat
gaaaataaaaacggcttgcttgttctcgatgagtgcggtacttggtttaatacccgttcttggaatgataaggaaagacagccgattattgattggt
ttctacatgctcgtaaattaggatgggatattatttttcttgttcaggacttatctattgttgataaacaggcgcgttctgcattagctgaacatgt
tgtttattgtcgtcgtctggacagaattactttaccttttgtcggtactttatattctcttattactggctcgaaaatgcctctgcctaaattacat
gttggcgttgttaaatatggcgattctcaattaagccctactgttgagcgttggctttatactggtaagaatttgtataacgcatatgatactaaac
aggctttttctagtaattatgattccggtgtttattcttatttaacgccttatttatcacacggtcggtatttcaaaccattaaatttaggtcagaa
gatgaaattaactaaaatatatttgaaaaagttttctcgcgttctttgtcttgcgattggatttgcatcagcatttacatatagttatataacccaa
cctaagccggaggttaaaaaggtagtctctcagacctatgattttgataaattcactattgactcttctcagcgtcttaatctaagctatcgctatg
ttttcaaggattctaagggaaaattaattaatagcgacgatttacagaagcaaggttattcactcacatatattgatttatgtactgtttccattaa
aaaaggtaattcaaatgaaattgttaaatgtaattaattttgttttcttgatgtttgtttcatcatcttcttttgctcaggtaattgaaatgaataa
ttcgcctctgcgcgattttgtaacttggtattcaaagcaatcaggcgaatccaagctt.

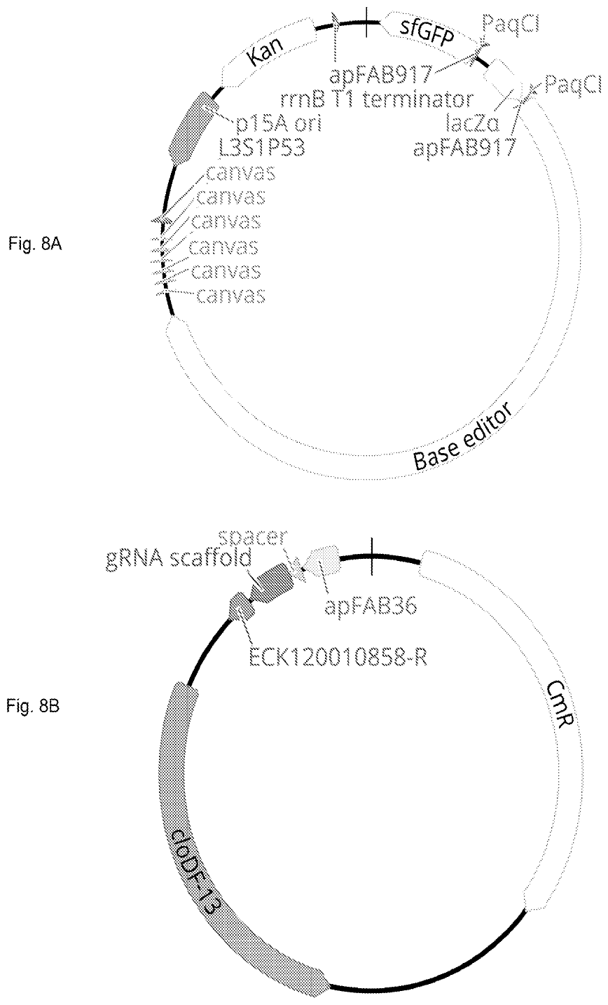

SEQ ID NO: 28 is sequence of vector plasmid in FIG. 8A

cgacactcactatagggagagcggcgtcgtaactagtagtgtcgtaaataaaaaaggcacgtcagatgacgtgccttttttcttgtgttagtgatg
gtggtggtgatggcttcccttgtagagttcgtccattccgtgagtaatgcctgcagcggtcacgaattctaacagaaccatgtgatcgcgttttt
cgttcggatctttgctcaggactgactgggtgctcaggtagtggttgtcaggtaacagtacaggcccgtcaccaatcggcgtgttttgttgata
atgatccgctaattgaacgctgccgtcttccacgttatggcgaattttgaaatttgccttgataccgtttttctgtttatcggccgtaatatacacg
ttatgagaattaaagttatattccagcttatggcctaaaatgttgccgtcttctttgaagtcgatacctttcagttcgatacggtttactaatgtgt
cgccttcaaatttcacttccgcacgcgttttataggtgccatcgtccttgaagctgattgtacgctcttgaacatatccttcaggcatggctgattt
gaaaaaatcgtgttgtttcatatgatctgggtaacgactgaagcactgaacgccataggtcagggtggtcactaaggtgggccacggaacgggta
atttgcctgttgtgcaaataaacttcagggtcaatttaccattggtagcgtcaccttcgccttcgccgcgcacgctaaatttgtgaccgttcacgt
cgccgtctaattccactaagatcggtactacacccgtaaacagctcttcacctttgctcatatgaagcaggtgtaatgtgagttagctcactcatt
aggcaccccaggctttacactttatgcttccggctcgtatgttgtgtggaattgtgagcggataacaatttcacacaggaaacagctatgacca
tgattacgccaagcttgcatgcctgcaggtcgactctagaggatccccgggtaccgagctcgaattcactggccgtcgttttacaacgtcgtg
actgggaaaaccctggcgttacccaacttaatcgccttgcagcacatccccctttcgccagctggcgtaatagcgaagaggcccgcaccga
tcgcccttcccaacagttgcgcagcctgaatggcgaatggcgcctgatgcggtattttctccttacgcatctgtgcggtatttcacaccgcatat
ggtgcactctcagtacaatctgctctgatgccgcatagcacctgcgaagatcttaatctagcggaggagactttcatatgtctaccgacgctga
atacgttcgtatccacgaaaaactggacatctacaccttcaaaaaacagttctctaacaacaaaaaatctgtttctcaccgttgctacgttctgtt
cgaactgaaacgtcgtggtgaacgtcgtgcttgcttctggggttacgctgttaacaaaccgcagtctggtaccgaacgtggtatccacgctga
aatcttctctatccgtaaagttgaagaatacctgcgtgacaacccgggtcagttcaccatcaactggtactcttcttggtccccgtgcgctgact
gcgctgaaaaaatcctggaatggtacaaccaggaactgcgtggtaacggtcacaccctgaagatatgggtctgcaagctgtactacgaaaa
aaacgctcgtaaccagatcggtctgtggaacctgcgtgacaacggtgttggtctgaacgttatggtttctgaacactaccagtgctgccgtaa
aatcttcatccagtcttctcacaaccagctgaacgaaaaccgttggctggaaaaaaccctgaaacgtgctgaaaaacgtcgttctgaactgtc
tatcatgttccaggttaaaatcctgcacaccaccaaatcctccggctgttagcggcggttcttccggtggctcctctggttctgaaaccccgggt
acctctgaatctgctaccccggaatctagcggtggctcctctggcggttctgataagaaatactcaataggcttagctatcggcacaaatagc
gtcggatgggcggtgatcactgatgaatataaggttccgtctaaaaagttcaaggttctgggaaatacagaccgccacagtatcaaaaaaaa
tcttataggggctcttttatttgacagtggagagacagcggaagcgactcgtctcaaacggacagctcgtagaaggtatacacgtcggaaga
atcgtatttgttatctacaggagattttttcaaatgagatggcgaaagtagatgatagtttctttcatcgacttgaagagtcttttttggtggaaga
agacaagaagcatgaacgtcatcctatttttggaaatatagtagatgaagttgcttatcatgagaaatatccaactatctatcatctgcgaaaaaa
attggtagattctactgataaagcggatttgcgcttaatctatttggccttagcgcatatgattaagtttcgtggtcattttttgattgagggagat
ttaaatcctgataatagtgatgtggacaaactatttatccagttggtacaaacctacaatcaattatttgaagaaaaccctattaacgcaagtggag
tagatgctaaagcgattctttctgcacgattgagtaaatcaagacgattagaaaatctcattgctcagctccccggtgagaagaaaaatggctt
atttgggaatctcattgctttgtcattgggtttgacccctaattttaaatcaaattttgatttggcagaagatgctaaattacagctttcaaaagat
acttacgatgatgatttagataatttattggcgcaaattggagatcaatatgctgatttgtttttggcagctaagaatttatcagatgctattttac
tttcagatatcctaagagtaaatactgaaataactaaggctcccctatcagcttcaatgattaaacgctacgatgaacatcatcaagacttgactct
tttaaaagctttagttcgacaacaacttccagaaaagtataaagaaatcttttttgatcaatcaaaaaacggatatgcaggttatattgatggggga
gctagccaagaagaattttataaatttatcaaaccaattttagaaaaaatggatggtactgaggaattattggtgaaactaaatcgtgaagatttgc
tgcgcaagcaacggacctttgacaacggctctattccccatcaaattcacttgggtgagctgcatgctattttgagaagacaagaagacttttat
ccatttttaaaagacaatcgtgagaagattgaaaaaatcttgacttttcgaattccttattatgttggtccattggcgcgtggcaatagtcgttttg
catggatgactcggaagtctgaagaaacaattaccccatggaattttgaagaagttgtcgataaaggtgcttcagctcaatcatttattgaacgc
atgacaaactttgataaaaatcttccaaatgaaaaagtactaccaaaacatagtttgctttatgagtattttacggtttataacgaattgacaaagg
tcaaatatgttactgaaggaatgcgaaaaccagcatttctttcaggtgaacagaagaaagccattgttgatttactcttcaaaacaaatcgaaaa
gtaaccgttaagcaattaaaagaagattatttcaaaaaaatagaatgttttgatagtgttgaaatttcaggagttgaagatagatttaatgcttcat
taggtacctaccatgatttgctaaaaattattaaagataaagattttttggataatgaagaaaatgaagatatcttagaggatattgttttaacatt
gaccttatttgaagatagggagatgattgaggaaagacttaaaacatatgctcacctctttgatgataaggtgatgaaacagcttaaacgtcgccg
ttatactggttggggacgtttgtctcgaaaattgattaatggtattaggataagcaatctggcaaaacaatattagattttttgaaatcagatggtt
ttgccaatcgcaattttatgcagctgatccatgatgatagtttgacatttaaagaagacattcaaaaagcacaagtgtctggacaaggcgatagt
ttacatgaacatattgcaaatttagctggtagccctgctattaaaaaaggtattttacagactgtaaaagttgttgatgaattggtcaaagtaatgg
ggcggcataagccagaaaatatcgttattgaaatggcacgtgaaaatcagacaactcaaaagggccagaaaaattcgcgagagcgtatga
aacgaatcgaagaaggtatcaaagaattaggaagtcagattcttaaagagcatcctgttgaaaatactcaattgcaaaatgaaaagctctatct
ctattatctccaaaatggaagagacatgtatgtggaccaagaattagatattaatcgtttaagtgattatgatgtcgatcacattgttccacaaagt
ttccttaaagacgattcaatagacaataaggtcttaacgcgttctgataaaaatcgtggtaaatcggataacgttccagtgaagaagtagtca
aaaagatgaaaaactattggagacaacttctaaacgccaagttaatcactcaacgtaagtttgataatttaacgaaagctgaacgtggaggttt
gagtgaacttgataaagctggttttatcaaacgccaattggttgaaactcgccaaatcactaagcatgtggcacaaattttggatagtcgcatg
aatactaaatacgatgaaaatgataaacttattcgagaggttaaagtgattaccttaaaatctaaattagtttctgacttccgaaaagatttccaat
tctataaagtacgtgagattaacaattaccatcatgcccatgatgcgtatctaaatgccgtcgttggaactgctttgattaagaaatatccaaaact
tgaatcggagtttgtctatggtgattataaagtttatgatgttcgtaaaatgattgctaagtctgagcaagaaataggcaaagcaaccgcaaaat
atttcttttactctaatatcatgaacttcttcaaaacagaaattacacttgcaaatggagagattcgcaaacgccctctaatcgaaactaatgggg
aaactggagaaattgtctgggataaagggcgagattttgccacagtgcgcaaagtattgtccatgccccaagtcaatattgtcaagaaaaca
gaagtacagacaggcggattctccaaggagtcaattttaccaaaaagaaattcggacaagcttattgctcgtaaaaaagactgggatccaaa
aaaatatggtggttttgatagtccaacggtagcttattcagtcctagtggttgctaaggtggaaaaagggaaatcgaagaagttaaaatccgtt
aaagagttactagggatcacaattatggaaagaagttcctttgaaaaaaatccgattgactttttagaagctaaaggatataaggaagttaaaa
aagacttaatcattaaactacctaaatatagtctttttgagttagaaaacggtcgtaaacggatgctggctagtgccggagaattacaaaaagg
aaatgagctggctctgccaagcaaatatgtgaattttttatatttagctagtcattatgaaaagttgaagggtagtccagaagataacgaacaaa
aacaattgtttgtggagcagcataagcattatttagatgagattattgagcaaatcagtgaattttctaagcgtgttattttagcagatgccaattt
agataaagttcttagtgcatataacaaacatagagacaaaccaatacgtgaacaagcagaaaatattattcatttatttacgttgacgaatcttgga
gctcccgctgcttttaaatattttgatacaacaattgatcgtaaacgatatacgtctacaaaagaagttttagatgccactcttatccatcaatcca
tcactggtctttatgaaacacgcattgatttgagtcagctaggaggtgacagcggcggtagcggcggtagcggtggtagcactaacctgagc
gacatcattgagaaggagactggtaaacagctggttattcaggagtccatcctgatgctgccggaggaggtggaggaagtgatcggcaac
aagccagagtctgacatcctggtgcaaccgcctacgacgagtccaccgatgagaacgtgatgcttctgacctctgacgccccggagtata
agccgtgggccctggttatccaggattctaacggcgagaacaagatcaagatgctgagcggtggttccggtggttctggtggtagcaccaa
cctgtctgacatcatcgagaaggagacgggcaagcagctggttattcaggagtccatcctgatgctgccggaggaggtggaggaagtgat
cggcaacaagccagagtctgacatcctggtgcacaccgcctacgacgagtccaccgatgagaacgtgatgcttctgacctctgacgcccc
ggagtataagccgtgggccctggttatccaggattccaacggtgagaacaaaatcaaaatgctgtaacctcggtaccaaattccagaaaag
aggcctcccgaaaggggggccttttttcgttttggtccacgaaaaaggcgcgccattaatcatccggaatgcaccatacacatatgtctcaag
tgaggtagcaacatgacaattcttgtggctggctcgaattgcccagaaccgcacccaccgaggtcacttctctgttggcacgaaaagggca
ataagatttacggattactatcgccagaaccgcacccaccgtggtgaaaagcaatccctcgtgaagtaactcaatagtgttctctggtatcgt
agcccagaaccgcacccaccggggttacaatcagcagtcagaacttttacgaagaatagtggtcgctcaaccttgcccagaaccgcaccc
accggggtgaacgacactactatttcttacgagatacttattctggaagcaacggtgcccagaaccgcacccaccgtggtattcacttccctc
acagattcgttcagagataaaagcgttggtaacagtgcccagaaccgcacccaccgaggctggaatacagataaggatagcgtcgttaca
atagtcactcgtagaactttaagatacaatggtaaccacaagaattgcaatgaccatgccacattacataacccaattattgaaggcctcccaa
atcggggggccttttttattgataacaaaaacgaagacggcgcgagaccacagtgactgcatgctagcggtctctacgatacagcggccg
ctgtagcctgccatggaaaatcgatgttcttaggctaggtggaggctcagtgatgataagtctgcgatggtggatgcatgtgtcatggtcatag
ctgtttcctgtgtgaaattgttatccgctcagagggcacaatcctattccgcgctatccgacaatctccaagacattaggtggagttcagttcgg
cgagcggaaatggcttacgaacggggcggagatttcctggaagatgccaggaagatacttaacagggaagtgagagggccgcggcaaa
gccgtttttccataggctccgccccctgacaagcatcacgaaatctgacgctcaaatcagtggtggcgaaacccgacaggactataaagat
accaggcgtttccccctggcggctccctcgtgcgctctcctgttcctgcctttcggtttaccggtgtcattccgctgttatggccgcgtttgtctc
attccacgcctgacactcagttccgggtaggcagttcgctccaagctggactgtatgcacgaaccccccgttcagtccgaccgctgcgcctt
atccggtaactatcgtcttgagtccaacccggaaagacatgcaaaagcaccactggcagcagccactggtaattgatttagaggagttagtc
ttgaagtcatgcgccggttaaggctaaactgaaaggacaagttttggtgactgcgctcctccaagccagttacctcggttcaaagagttggta
gctcagagaaccttcgaaaaaccgccctgcaaggcggttttttcgttttcagagcaagagattacgcgcagaccaaaacgatctcaagaag
atcatcttattaagtctgacgctctattcaacaaagccgccgtccatgggtagggggcttcaaatcgtccgctctgccagtgttacaaccaatta
acaaattctgattagaaaaactcatcgagcatcaaatgaaactgcaatttattcatatcaggattatcaataccatatttttgaaaaagccgtttct
gtaatgaaggagaaaactcaccgaggcagttccataggatggcaagatcctggtatcggtcgcgattccgactcgtccaacatcaatacaa
cctattaatttcccctcgtcaaaaataaggttatcaagtgagaaatcaccatgagtgacgactgaatccggtgagaatggcaaaagcttatgca
tttctttccagacttgttcaacaggccagccattacgctcgtcatcaaaatcactcgcatcaaccaaaccgttattcattcgtgattgcgcctgag
cgagacgaaatacgcgatcgctgttaaaaggacaattacaaacaggaatcgaatgcaaccggcgcaggaacactgccagcgcatcaaca
atatttcacctgaatcaggatattcttctaatacctggaatgctgttttcccggggatcgcagtggtgagtaaccatgcatcatcaggagtacg
gataaaatgcttgatggtcggaagaggcataaattccgtcagccagtttagtctgaccatctcatctgtaacatcattggcaacgctacctttgc
catgtttcagaaacaactctggcgcatcgggcttcccatacaatcgatagattgtcgcacctgattgcccgacattatcgcgagcccatttata
cccatataaatcagcatccatgttggaatttaatcgcggcctcgagcaagacgtttcccgttgaatatggctcataacaccccttgtattactgtt
tatgtaagcagacagttttattgttcatgatgatatatttttatcttgtgcaatgtaacatcagagattttgagacacaacgtggctttcccccgcc
gctctagaactagtggatccaaataaaacgaaaggctcagtcgaaagactgggcctttcgttttatctgttgtttgtcgcattatacgagacgtcc
aggttgggatacctgaaacaaaacccatcgtacggccaaggaagtctccaataactgtgatccaccacaagcgccagggttttcccagtca
cgacgttgtaaaacgacggccagtcatgcataatccgcacgcatctggaataaggaagtgccattccgcctgacct.

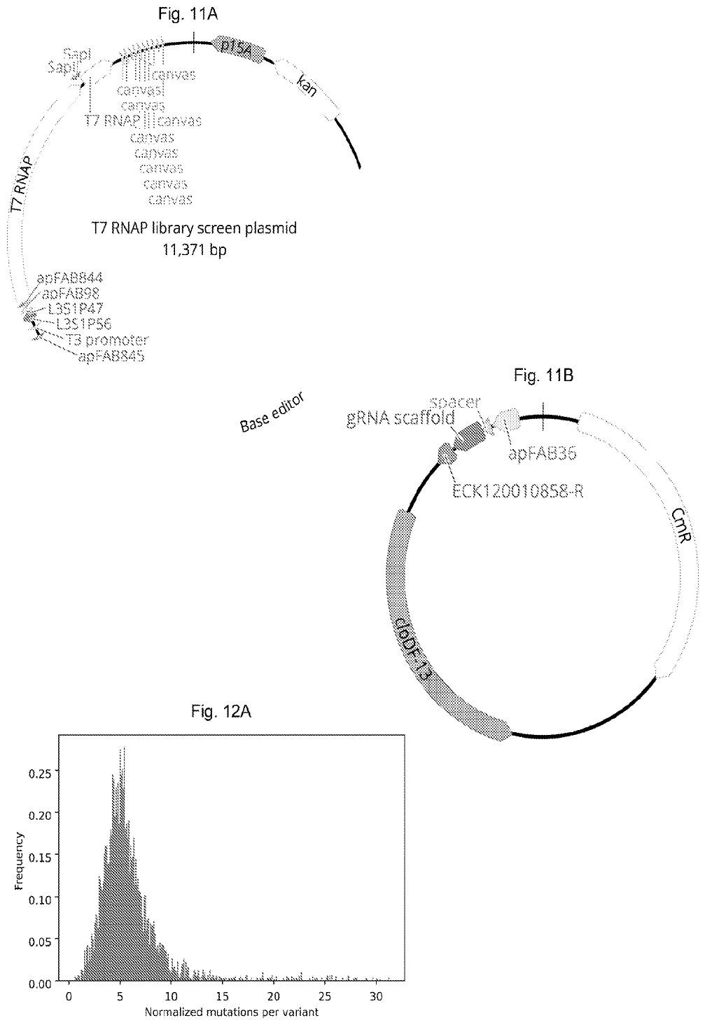

SEQ ID NO: 29 is sequence of sgRNA expression plasmid in FIGS. 8B and 11B

tccaactttcaccataatgaaataagatcactaccgggcgtatttttgagttatcgagattttcaggagctaaggaagctaaaatggagaaaaa
aatcactggatataccaccgttgatatatcccaatggcatcgtaaagaacattttgaggcatttcagtcagttgctaaatgtacctataaccaga
ccgttcagctggatattacggccttttaaagaccgtaaagaaaaataagcacaagttttatccggcctttattcacattcttgcccgcctgatga
atgctcatccggagttccgtatggcaatgaaagacggtgagctggtgatatgggatagtgttcacccttgttacaccgttttccatgagcaaac
tgaaacgttttcatcgctctggagtgaataccacgacgatttccggcagtttctacacatatattcgcaagatgtggcgtgttacggtgaaaacc
tggcctatttccctaaagggtttattgagaatatgtttttcgtctcagccaatccctgggtgagtttcaccagttttgatttaaacgtggccaatat
ggacaacttcttcgcccccgttttcactatgggcaaatattatacgcaaggcgacaaggtgctgatgccgctggcgattcaggttcatcatgcc
gtttgtgatggcttccatgtcggcagaatgcttaatgaattacaacagtactgcgatgagtggcagggcggggcgtaaacgccatgggcatg
tagtcaaaagcctccggtcggaggcttttgacttggctgaggaagtgccgttaattaagtccgtggggaaaaaatcatggcaattctggaaga
aatagcgcctttcagccggcaaacctgaagccggatctgcgattctgataacaaactagcaacaccagaacagcccgtttgcgggcagcaa
aatagcgctttcagccggcaaacctgaagccggatctgcgattctgataacaaactagcaacaccagaacagcccgtttgcgggcagcaa
aacccgtaccctaggtctagggcggcggatttgtcctactcaggagagcgttcaccgacaaacagataaaacgaaaggcccagtcttt
cgactgagcctttcgttttatttgatgcctctagttgggcgcgccgggtgggcctttctgcgttgctggcgtttttccataggctccgcccccctg
acgagcatcacaaaaatcgatgctcaagtcagaggtggcgaaacccgacaggactataaagataccaggcgtttccccctggaagctccc
tcgtgcgctctcctgttccgaccctgccgcttaccggatacctgtccgcctttctcccttcgggaagcgtggcgctttctcatagctcacgctgt
aggtatctcagttcggtgtaggtcgttcgctccaagctgggctgtgtgcacgaaccccccgttcagcccgaccgctgcgccttatcctgtaac
tatcgtcttgagtccaacccggtaagacacgacttatcgccactggcagcagccactggtaacaggattagcagagcgaggtatgtaggcg
gtgctacagagttcttgaagtggtggcctaactacggctacactagaagaacagtatttggtatctgcgctctgctgaagccagttacctcgga
aaaagagttggtagctcttgatccggcaaacaaaccaccgctggtagcggtggtttttttgtttgcaagcagcagattacgcgcagaaaaaaa
ggatctcaagaagatcctttgattttctaccgaagaaagacccacccgtgaaggtgagccagtgagttgattgcagtccagttacgctggagt
caagactagtcgtctccacctgcatacagtgacgaccgtaataaaaaaggcacgtcagatgacgtgccttttttcttgtgttagcaccgactcg
gtgccactttttcaagttgataacggactagccttattttaacttgctatttctagctctaaaaccggtgggtgcggttctgggcagatcttcaatg
aatctattatacgagccggatgattaatagtcaatactctttttggcgcgccctaaccaggataagcaggtgatactgagacggcggccgcg
gcacgtaagaggt.

SEQ ID NO: 30 is sequence of Vector plasmid in FIG. 11A

tgtgaaattgttatccgctcagagggcacaatcctattccgcgctatccgacaatctccaagacattaggtggagttcagttcggcgagcgga
aatggcttacgaacggggcggagatttcctggaagatgccaggaagatacttaacagggaagtgagagggccgcggcaaagccgtttttc
cataggctccgcccccctgacaagcatcacgaaatctgacgctcaaatcagtggtggcgaaacccgacaggactataaagataccaggcg
tttccccctggcggctccctcgtgcgctctcctgttcctgcctttcggtttaccggtgtcattccgctgttatggccgcgtttgtctcattccacgc
ctgacactcagttccgggtaggcagttcgctccaagctggactgtatgcacgaaccccccgttcagtccgaccgctgcgccttatccggtaa
ctatcgtcttgagtccaacccggaaagacatgcaaaagcaccactggcagcagccactggtaattgatttagaggagttagtcttgaagtcat
gcgccggttaaggctaaactgaaaggacaagttttggtgactgcgctcctccaagccagttacctcggttcaaagagttggtagctcagaga
accttcgaaaaaccgccctgcaaggcggttttttcgttttcagagcaagagattacgcgcagaccaaaacgatctcaagaagatcatcttatta
agtctgacgctctattcaacaaagccgccgtccatgggtagggggcttcaaatcgtccgctctgccagtgttacaaccaattaacaaattctg
attagaaaaactcatcgagcatcaaatgaaactgcaatttattcatatcaggattatcaataccatatttttgaaaaagccgtttctgtaatgaagg
agaaaactcaccgaggcagttccataggatggcaagatcctggtatcggtctgcgattccgactcgtccaacatcaatacaacctattaatttc
ccctcgtcaaaaataaggttatcaagtgagaaatcaccatgagtgacgactgaatccggtgagaatggcaaaagcttatgcatttctttccag
acttgttcaacaggccagccattacgctcgtcatcaaaatcactcgcatcaaccaaaccgttattcattcgtgattgcgcctgagcgagacga
aatacgcgatcgctgttaaaaggacaattacaaacaggaatcgaatgcaaccggcgcaggaacactgccagcgcatcaacaatattttcac
ctgaatcaggatattcttctaatacctggaatgctgttttcccggggatcgcagtggtgagtaaccatgcatcatcaggagtacggataaaatg
cttgatggtcggaagaggcataaattccgtcagccagtttagtctgaccatctcatctgtaacatcattggcaacgctacctttgccatgtttcag
aaacaactctggcgcatcgggcttcccatacaatcgatagattgtcgcacctgattgcccgacattatcgcgagcccatttatacccatataaa
tcagcatccatgttggaatttaatcgcggcctcgagcaagacgtttcccgttgaatatggctcataacaccccttgtattactgtttatgtaagca
gacagttttattgttcatgatgatatatttttatcttgtgcaatgtaacatcagagattttgagacacaacgtggctttcccccgccgctctagaac
tagtggatccaaataaaacgaaaggctcagtcgaaagactgggcctttcgttttatctgttgtttgtcgcattatacgagacgtccaggttgggat
acctgaaacaaaacccatcgtacggccaaggaagtctccaataactgtgatccaccacaagcgccagggttttcccagtcacgacgttgta
aaacgacggccagtgtacttcgttcagtcttgtgtcccagttaccagggttgtaaaacgacggccagtcatgcataatccgcacgcatctgga
ataaggaagtgccattccgcctgacctcgactcactatagggagagcggcgtcgtaactagtagtgtcgtaaataaaaaaggcacgtcaga
tgacgtgccttttttcttgtgttacagcattttgattttgttctcaccgttggaatcctggataaccagggcccacggcttatactccggggcgtca
gaggtcagaagcatcacgttctcatcggtggactcgtcgtaggcggtgtgcaccaggatgtcagactctggcttgttgccgatcacttcctcc
acctcctccggcagcatcaggatggactcctgaataaccagctgcttgcccgtttccttctcgatgatgtcagacaggttggtgctaccacca
gaaccaccggaaccaccgctcagcatcttgatcttgttctcgccgttagaatcctggataaccagggcccacggcttatactccggggcgtc
agaggtcagaagcatcacgttctcatcggtggactcgtcgtaggcggtgtgcaccaggatgtcagactctggcttgttgccgatcacttcctc
cacctcctccggcagcatcaggatggactcctgaataaccagctgtttaccagtctccttctcaatgatgtcgctcaggttagtgctaccaccg
ctaccgccgctaccgccgctgtcacctcctagctgactcaaatcaatgcgtgtttcataaagaccagtgatggattgatggataagagtggca
tctaaaacttcttttgtagacgtatatcgtttacgatcaattgttgtatcaaaatatttaaaagcagcgggagctccaagattcgtcaacgtaaata
aatgaataatattttctgcttgttcacgtattggtttgtctctatgtttgttatatgcactaagaactttatctaaattggcatctgctaaaataac
acgcttagaaaattcactgatttgctcaataatctcatctaaataatgcttatgctgctccacaaacaattgtttttgttcgttatcttctggacta
cccttcaacttttcataatgactagctaaatataaaaaattcacatatttgcttggcagagccagctcatttcctttttgtaattctccggcactag
ccagcatccgtttacgaccgttttctaactcaaaaagactatatttaggtagtttaatgattaagtcttttttaacttccttatatcctttagcttc
taaaaagtcaatcggatttttttcaaaggaacttctttccataattgtgatccctagtaactctttaacggattttaacttcttcgatttccctttt
tccaccttagcaaccactaggactgaataagctaccgttggactatcaaaaccaccatatttttttggatcccagtcttttttacgagcaataagct
tgtcccgaatttctttttggtaaaattgactccttggagaatccgcctgtctgtacttctgttttcttgacaatattgacttggggcatggacaata
ctttgcgcactgtggcaaaatctcgccctttatcccagacaatttctccagtttccccattagtttcgattagagggcgtttgcgaatctctccatt
tgcaagtgtaatttctgttttgaagaagttcatgatattagagtaaaagaaatattttgcggttgctttgcctatttcttgctcagacttagcaatc
attttacgaacatcataaactttataatcaccatagacaaactccgattcaagttttggatatttcttaatcaaagcagttccaacgacggcattta
gatacgcatcatgggcatgatggtaattgttaatctcacgtactttatagaattggaaatcttttcggaagtcagaaactaatttagattttaaggt
aatcactttaacctctcgaataagtttatcattttcatcgtatttagtattcatgcgactatccaaaatttgtgccacatgcttagtgatttggcga
gtttcaaccaattggcgtttgataaaaccagctttatcaagttcactcaaacctccacgttcagctttcgttaaattatcaaacttacgttgagtga
ttaacttggcgtttagaagttgtctccaatagtttttcatctttttgactacttcttcacttggaacgttatccgatttaccacgatttttatcaga
acgcgttaagaccttattgtctattgaatcgtctttaaggaaactttgtggaacaatgtgatcgacatcataatcacttaaacgattaatatctaat
tcttggtccacatacatgtctcttccattttggagataatagagatagagcttttcattttgcaattgagtattttcaacaggatgctctttaagaa
tctgacttcctaattctttgataccttcttcgattcgtttcatacgctctcgcgaatttttctggcccttttgagttgtctgattttcacgtgccat
ttcaataacgatattttctggcttatgccgccccattactttgaccaattcatcaacaacttttacagtctgtaaaataccttttttaatagcaggg
ctaccagctaaatttgcaatatgttcatgtaaactatcgccttgtccagacacttgtgctttttgaatgtcttctttaaatgtcaaactatcatcat
ggatcagctgcataaaattgcgattggcaaaaccatctgatttcaaaaaatctaatattgttttgccagattgcttatccctaataccattaatcaa
ttttcgagacaaacgtccccaaccagtataacggcgacgtttaagctgtttcatcaccttatcatcaaagaggtgagcatatgttttaagtctttcc
tcaatcatctccctatcttcaaataaggtcaatgttaaaacaatatcctctaagatatcttcattttcttcattatccaaaaaatctttatctttaa
taatttttagcaaatcatggtaggtacctaatgaagcattaaatctatcttcaactcctgaaatttcaacactatcaaaacattctattttttgaaa
taatcttcttttaattgcttaacggttacttttcgatttgttttgaagagtaaatcaacaatggctttcttctgttcacctgaaagaaatgctggtt
ttcgcattccttcagtaacatatttgacctttgtcaattcgttataaaccgtaaaatactcataaagcaaactatgttttggtagtactttttcatt
tggaagatttttatcaaagtttgtcatgcgttcaataaatgattgagctgaagcacctttatcgacaacttcttcaaaattccatggggtaattgtt
tcttcagacttccgagtcatccatgcaaaacgactattgccacgcgccaatggaccaacataataaggaattcgaaaagtcaagattttttcaatct
tctcacgattgtcttttaaaaatggataaaagtcttcttgtcttctcaaaatagcatgcagctcacccaagtgaatttgatggggaatagagccgtt
gtcaaaggtccgttgcttgcgcagcaaatcttcacgatttagtttcaccaataattcctcagtaccatccattttttctaaaattggtttgataaat
ttataaaattcttcttggctagctcccccatcaatataacctgcatatccgtttttgattgatcaaaaaagatttctttatacttttctggaagttg
ttgtcgaactaaagcttttaaaagagtcaagtcttgatgatgttcatcgtagcgtttaatcattgaagctgataggggagccttagttatttcagta
tttactcttaggatatctgaaagtaaaatagcatctgataaattcttagctgccaaaaacaaatcagcatattgatctccaatttgcgccaataaat
tatctaaatcatcatcgtaagtatcttttgaaagctgtaatttagcatcttctgccaaatcaaaatttgatttaaaattaggggtcaaacccaatga
caaagcaatgagattcccaaataagccatttttcttctcaccggggagctgagcaatgagattttctaatcgtcttgatttactcaatcgtgcagaa
agaatcgctttagcatctactccacttgcgttaatagggttttcttcaaataattgattgtaggtttgtaccaactggataaatagtttgtccacat
cactattatcaggatttaaatctccctcaatcaaaaaatgaccacgaaacttaatcatatgcgctaaggccaaatagattaagcgcaaatccgcttt
atcagtagaatctaccaatttttttcgcagatgatagatagttggatatttctcatgataagcaacttcatctactatatttccaaaaataggatga
cgttcatgcttcttgtcttcttccaccaaaaaagactcttcaagtcgatgaaagaaactatcatctactttcgccatctcatttgaaaaaatctcct
gtagataacaaatacgattcttccgacgtgtataccttctacgagctgtccgtttaagacgagtcgcttccgctgtctctccactgtcaaataaaag
agcccctataagattttttttgatactgtggcggtctgtatttcccagaaccttgaactttttagacggaaccttatattcatcagtgatcaccgcc
catccgacgctatttgtgccgatagctaagcctattgagtatttcttatcagaaccgccagaggagccaccgctagattccggggtagcagattcag
aggtacccggggtttcagaaccagaggagccaccggaagaaccgccgctaacagccggagatttggtggtgtgcaggattttaacctggaacatgat
agacagttcagaacgacgtttttcagcacgtttcagggttttttccagccaacggttttcgttcagctggttgtgagaagactggatgaagatttta
cggcagcactggtagtgttcagaaaccataacgttcagaccaacaccgttgtcacgcaggttccacagaccgatctggttacgagcgtttttttcgt
agtacagcttgcagacccatatcttcagggtgtgaccgttaccacgcagttcctggttgtaccattccaggattttttcagcgcagtcagcgcacgg
ggaccaagaagagtaccagttgatggtgaactgacccgggttgtcacgcaggtattcttcaactttacggatagagaagatttcagcgtggatacca
cgttcggtaccagactgcggtttgttaacagcgtaaccccagaagcaagcacgacgttcaccacgacgtttcagttcgaacagaacgtagcaacggt
gagaaacagattttttgttgttagagaactgttttttgaaggtgtagatgtccagtttttcgtggatacgaacgtattcagcgtcggtagacatatg
aaagtctccccggctagattaagataaagttaaacaaaattatttgtagagggaaaccgttgttgtctcccagatactgcggctgcaggtctttctc
cctttagtgagggttaattcgcccaggggcgcgccattaatcatccggaatgcaccatacacatatgttttgttatcaataaaaaaggcccccgatt
tgggaggccttttttcgaaaataacttttcgaaaaaaggcctcccaaatcgggggccttttttatagcaacaaaaacgaactcggcgcgccaaaaaa
tttatttgctttcgcatctttttgtacctagatttaacgtatcccgaatcttaatctagcaggggacactttcatatgaacacgattaacatcgcta
agaacgacttctctgacatcgaactggctgctatcccgttcaacactctggctgaccattacggtgagcgtttagctcgcgaacagttggcccttga
gcatgagtcttacgagatgggtgaagcacgcttccgcaagatgtttgagcgtcaacttaaagctggtgaggttgcggataacgctgccgctaagcct
ctcatcactaccctactccctaagatgattgcacgcatcaacgactggtttgaggaagtgaaagctaagcgcggcaagcgcccgacagccttccagt
tcctgcaagaaatcaagccggaagccgtagcgtacatcacaattaagaccactctggcttgcctaaccagtgctgacaatacaaccgttcaggctgt
agcaagcgcaatcggtcgggccattgaggacgaggctcgcttcggtcgtatccgtgaccttgaagctaagcacttcaagaaaaacgttgaggaacaa
ctcaacaagcgcgtagggcacgtctacaagaaagcatttatgcaagttgtcgaggctgacatgctctctaagggtctactcggtggcgaggcgtg
gtcttcgtggcataaggaagactctattcatgtaggagtacgctgcatcgagatgctcattgagtcaaccggaatggttagcttacaccgcca
aaatgctggcgtagtaggtcaagactctgagactatcgaactcgcacctgaatacgctgaggctatcgcaacccgtgctggtgcgctggct
ggcatctctccgatgttccaaccttgcgtagttcctcctaagccgtggactggcattactggtggtggctattgggctaacggtcgtcgtcctct
ggcgctggtgcgtactcacagtaagaaagcactgatgcgctacgaagacgtttacatgcctgaggtgtacaaagcgattaacattgcgcaa
aacaccgcatggaaaatcaacaagaaagtcctagcggtcgccaacgtaatcaccaagtggaagcattgtccggtcgaggacatccctgcg
attgagcgtgaagaactcccgatgaaaccggaagacatcgacatgaatcctgaggctctcaccgcgtggaaacgtgctgccgctgctgtgt
accgcaaggacaaggctcgcaagtctcgccgtatcagccttgagttcatgcttgagcaagccaataagtttgctaaccataaggccatctgg
ttcccttacaacatggactggcgcggtcgtgtttacgctgtgtcaatgttcaacccgcaaggtaacgatatgaccaaaggactgcttacgctg
gcgaaaggtaaaccaatcggtaaggaaggttactactggctgaaaatccacggtgcaaactgtgcgggtgtcgataaggttccgttccctg
agcgcatcaagttcattgaggaaaaccacgagaacatcatggcttgcgctaagtctccactggagaacacttggtgggctgagcaagattct
ccgttctgcttccttgcgttctgctttgagtacgctggggtacagcaccacggcctgagctataactgctcccttccgctggcgtttgacgggtc
ttgctctggcatccagcacttctccgcgatgctccgagatgaggtaggtggtcgcgcggttaacttgcttcctagtgaaaccgttcaggacat
ctacgggattgttgctaagaaagtcaacgagattctacaagcagacgcaatcaatgggaccgataacgaagtagttaccgtgaccgatgag
aacactggtgaaatctctgagaaagtcaagctgggcactaaggcactggctggtcaatggctggcttacggtgttactcgcagtgtgactaa
gcgttcagtcatgacgctggcttacgggtccaaagagttcggcttccgtcaacaagtgctggaagataccattcagccagctattgattccgg
caagggtctgatgttcactcagccgaatcaggctgctggatacatggctaagctgatttgggaatctgtgagcgtgacggtggtagctgcgg
ttgaagcaatgaactggcttaagtctgctgctaagctgctggctgctgaggtcaaagataagaagactggagagattcttcgcaagcgttgc
gctgtgcattgggtaactcctgatggtttccctgtgtggcaggaatacagaagagcaagcaatggaactgctcttcaaaagatagcgagattg
atgcacacaaacaggagtctggtatcgctcctaactttgtacacagccaagacggtagccaccttcgtaagactgtagtgtgggcacacgag
aagtacggaatcgaatcttttgcactgattcacgactccttcggtaccattccggctgacgctgcgaacctgttcaaagcagtgcgcgaaact
atggttgacacatatgagtcttgtgatgtactggctgatttctacgaccagttcgctgaccagttgcacgagtctcaattggacaaaatgccagc
acttccggctaaaggtaacttgaacctccgtgacatcttagagtcggacttcgcgttcgcgtaacacaagaaaaaggcacgtcatctgacgt
gccttttttatttacgaaaaaggcgcgccattaatcatccggaatgcaccatacacatatggcccagaaccgcacccaccgtggtactttcag
acgagaaaacgaaacggcccagaaccgcacccaccgtggttccgggacgcattttaaagaagaggcccagaaccgcacccaccgggg
aacagaggcctagtccgatgcatgtgcccagaaccgcacccaccgtggtcgcccctagaaacgaggggtccctgcccgaaccgcacc
caccgaggactattatgagcgtttaagtacattgcccagaaccgcacccaccgtggacgttaatatattatggacgcccgcgcccagaacc
gcacccaccggggattgttaggtagctaaacattacgtgcccagaaccgcacccaccgaggacaaatacaattaagaaagtctcgcgccc
agaaccgcacccaccggggggccagtcccaccagcgcggagtaagcccagaaccgcacccaccgtgggtaataatgattaaggtcacc
aggttaacccaattattgaaggcctcccaaatcggggggccttttttattgataacaaaaacgaagacggcgcgagacccacagtgactgca
tgctagcggtctctacgatacagcggccgctgtagcctgccatggaaaatcgatgttcttaggctaggtggaggctcagtgatgataagtctg
cgatggtggatgcatgtgtcatggtcatagctgtttcctgtccgattctgcttctttctacctgagcaatacgtcatagctgtttcctg.

### DETAILED DESCRIPTION

Certain aspects of the invention include methods for recoding the magnitude of a molecular activity associated with a nucleic acid into a sequence contiguous with that nucleic acid, thereby allowing the genotype and associated phenotype to be determined in a single sequencing step. Using methods of the invention the activity of a gene-encoding biomolecule is linked to a molecular recorder, thereby permitting measurement of activity of the biomolecule itself. Embodiments of the molecular recorders set forth herein can be used both in high-throughput methods as well as at the single cell level. Methods and systems of the invention may be referred to herein as direct high-throughput activity recording and measurement assay (DHARMA).

Certain methods of the invention can replace use of an optically active marker gene with a molecular recorder that introduces mutations into a sequence contiguous with the gene of interest. This allows the method to be used to ascertain the genotype and measure the phenotype in a single sequencing step. Certain embodiments of the invention include in high-throughput methods, and some embodiments of the invention are applied at the single-molecule level, by physically separating a library of sequences into unique compartments for the recording step, as can be achieved by transforming the sequences into cells or generating bubbles of water within an oil emulsion. Because mutations can accumulate over an extended period, allowing the molecular recorder to run for an extended period can arbitrarily increase the sensitivity beyond that achievable using optical methods. In contrast, a fluorescent-assisted cell sorter (flow cytometry-based, FACS) can only sort molecules into approximate bins of activity for later sequencing, the invention as described herein can be used to obtain an individual measurement for the activity of the nucleic acid sequence in each cell or compartment in an emulsion. Overall, methods and composition described herein can be used to entirely replace FACS for most directed evolution experiments.

The invention, in part, includes compositions, which may comprise: (i) a preselected gene of interest contiguous to a canvas polynucleotide sequence and (ii) a polynucleotide sequence encoding a mutagenic protein, wherein, when expressed, activity of the encoded mutagenic protein accumulates a detectable mutation in the canvas polynucleotide sequence at a rate proportional to a molecular activity of the expression product of the preselected gene of interest. Additional information rand detail regarding components and use of compositions of the invention are provided elsewhere herein.

Some embodiments of methods of the invention include encoding a canvas for highly efficiently molecular recording contiguous with a nucleic acid sequence responsible for generating the informational signal to be detected such that identity of the sequence and its activity are linked; reading the linked identity and activity information with a single sequencing read; extending the time of recording to increase sensitivity of measurement; and performing the above steps for many related sequences at once to map the relationship between phenotype and genotype. Certain embodiments of modular recording methods of the invention can be applied to screen libraries of sequences when performing protein optimization or directed evolution, and also for generating datasets linking genotype and associated phenotype for machine learning.

As used herein, the term “molecular recorder” means a genetic circuit that transforms a detectable informational signal into increased targeted or untargeted mutagenesis of nucleic acid sequences, such that the level of exposure to the informational signal can be determined by sequencing the nucleic acids. A composition of the invention, in a TTS environment is considered to be a genetic circuit. The term “gene of interest” as used herein means a nucleic acid sequence associated with a molecular activity of interest. The term “canvas” as used herein is a nucleic acid sequence contiguous with a gene of interest wherein the nucleic acid sequence accumulates mutations proportional to the molecular activity of the gene of interest, such that the identity of the gene of interest and its associated level of molecular activity can be determined in a single sequencing step. As used herein the term “reporter cell” means a cell comprising a composition of the invention. As used herein the term “plurality” means more than one, and so may mean 2, 3, 4, 5, 6, 7, 8, 9, 10, or more.

In some embodiments of the invention, a molecular recorder is prepared and/or used to determine the sequence and activity of a protein encoded by a preselected gene of interest. The use of a molecular recorder as described herein permits rapid, single-step determination of the nucleic acid sequence of a preselected gene of interest and an activity of its encoded protein. Methods of the invention may include preparing and use of a composition comprising (1) a preselected gene of interest, (2) a canvas polynucleotide sequence, and (3) a polynucleotide sequence encoding a mutagenic protein. In some embodiments of methods of the invention, certain elements of the composition are positioned in the composition such that the sequence of the preselected gene of interest is contiguous with the canvas polynucleotide sequence, and as a result, when expressed activity of the encoded mutagenic protein accumulates a detectable mutation in the canvas polynucleotide sequence at a rate that is proportional to a molecular activity of the expression product of the preselected gene of interest, thereby allowing the identity of the gene and its phenotypic activity to be determined in a single sequencing step.

### Preselected Gene of Interest

Certain embodiments of methods of the invention can be used to determine a sequence and an activity of a preselected gene of interest. A preselected gene of interest can be a gene or portion of a gene that is selected for assessment using a method of the invention. For example, though not intended to be limiting, a preselected gene of interest may be a gene encoding protein that when expressed has an activity or function. A non-limiting example is a gene encoding: an oxidoreductase, a transferase, a hydrolase, a lyase, an isomerase, a ligase (all six major enzyme classes), a DNA-binding protein, an RNA-binding protein, a protein-binding protein, a lipid-binding protein. Additional non-limiting examples of a gene of interest that may be preselected for assessment using a method of the invention is a gene encoding a recombinase, an integrase, a protease, a polymerase, a reverse transcriptase, a nuclease, a nickase, a tRNA, aminoacyl tRNA synthetase, or a ribosome. Compositions and/or methods of the invention may include other preselected genes of interest.

It will be understood that in some embodiments of methods of the invention, two or more different preselected genes of interest may be assessed using compositions and methods of the invention and in certain embodiments of methods of the invention two or more of a preselected gene of interest may be assessed using compositions and methods of the invention. Two or more of a preselected gene of interest can, but need not, have identical sequences, and the protein product of two or more preselected genes of interest can, but need not, have identical amino acid sequences. Sequence differences between two or more of a preselected gene of interest may result from natural sequence variation, a non-limiting example of which is different alleles of a gene; an engineered sequence variation, in which 1, 2, 3, 4, 5, or more sequence alterations such as substitutions, deletions, insertions, etc. are introduced into the nucleic acid sequence of the gene and in the amino acid sequence of the expression product of the preselected gene of interest. A non-limiting example of an engineered preselected gene of interest is a sequence prepared using a method of directed evolution [see for example Sarker, I., et al., Science (2007) June 29: 316(5833):1912-5; Badran, A. H., et al., Nature (2016) May 5; 533(7601): 58-63; and Blum, T. R., et al. Science (2021) February 19; 371(6531):803-810, the content of each of which is incorporated herein by reference in its entirety]. It will be understood that a preselected gene of interest may also include one or more spontaneously arising sequence changes and the gene is considered to be the preselected gene of interest.

### Canvas Polynucleotide

A canvas polynucleotide included in a composition of the invention may be a nucleic acid sequence that is positioned in a composition of the invention in a position contiguous with a preselected gene of interest. The canvas polynucleotide is selected so the molecular activity of the expressed gene of interest is proportional to, and can be measured by the accumulation of mutations in the canvas polynucleotide sequence. A canvas polynucleotide sequence included in a composition and/or method of the invention may be selected based on characteristics of the canvas polynucleotide sequence that permit mutations to accumulate in the sequence as a result of expression of the mutagenic protein encoded in the composition. For example, activity of the expressed mutagenic protein results in mutations in the sequence of the canvas polynucleotide sequence.

In some embodiments of the invention, a characteristic of mutations introduced into the canvas polynucleotide is determined and provides a measure of a level of activity of the expression product of the preselected gene of interest. Accumulation of a mutation introduced into a canvas polynucleotide sequence in a composition comprising a first preselected gene of interest may be compared to accumulation of the introduced mutation in the canvas polynucleotide sequence in a composition that includes (1) a different preselected gene of interest instead of the first preselected gene of interest; or (2) a variant of the first preselected gene of interest instead of the first preselected gene of interest. In methods of the invention one or more characteristics of mutations introduced into the canvas polynucleotide sequence in a composition of the invention can be determined as a measure of activity of the expression product of the preselected gene of interest that is included in the composition.

Non-limiting examples of a characteristic of a mutation introduced into a canvas polynucleotide sequence is a number of introduced mutations and a pattern of introduced mutations. A composition of the invention may be prepared such that a characteristic of one or more mutations introduced into the canvas polynucleotide sequence is relative to activity of the expressed gene of interest that is also included in the composition. Some embodiments of methods of the invention include determining a number of a mutation that have been introduced into the canvas polynucleotide sequence by the mutagenic protein that is encoded in the composition.

A canvas polynucleotide sequence included in methods and compositions of the invention may be a predetermined polynucleotide sequence. As used herein, the term “predetermined” used in reference to a polynucleotide sequences means a sequence that is selected, for one or more reasons by a practitioner of the invention. A non-limiting example of a reason for selecting a particular polynucleotide sequence is the sequence itself, the identity of one or more of the other sequences included in the composition of the invention, etc. A non-limiting example of a predetermined polynucleotide sequence that may be included in a canvas polynucleotide sequence is one or more of a: gI, gIV, and gVI sequence of an M13 bacteriophage. In certain embodiments a predetermined polynucleotide sequence of a canvas polynucleotide sequence comprises a repeat nucleic acid sequence, which is also referred to herein as a “repeated nucleic acid sequence”. A repeat nucleic acid sequence may comprise 2, 3, 4, 5, 6, 7, 8, 9, 10, 11, 12, 13, 14, 15, 16, 17, 18, 19 or 20 repeats of a preselected nucleic acid sequence. A non-limiting example of a repeated nucleic acid sequence that may be included in a canvas polynucleotide sequence of the invention is a TetO array. Repeats of the Tet operator sequence, which is bound by the tet repressor protein IetR in a manner dependent on the presence or absence of tetracycline-class compounds [see for example Das, A. T. et al., Cur Gene Ther. 2016 June; 16(3):156-167, the content of which is incorporated herein by reference in its entirety]. Including repeats of the Tet operator or any other repeat sequence within the canvas permits targeted mutation of all repeats, which can readily be detected by sequencing the gene of interest and all of the repeats.

In certain embodiments of compositions and methods of the invention, the canvas polynucleotide sequence comprises one or more guide RNA target sites for a base editor that is a mutagenic protein encoded into the composition. Certain embodiments of methods of the invention include expressing one or a plurality of gRNAs that are capable of directing the base editor to one or more target polynucleotide sequences that are present in the canvas polynucleotide sequence.

### Mutagenic Proteins

As described herein, certain embodiments of methods of the invention include a polynucleotide sequence encoding a mutagenic protein. A mon-limiting example of a mutagenic protein that may be used in an embodiment of the invention is an enzyme, wherein when the encoding nucleic acid is expressed, the resulting mutagenic protein is capable of acting to introduce a detectable mutation in the canvas polynucleotide sequence. A non-limiting example of a mutagenic protein that may be encoded in a composition of the invention is a deaminase, a terminal transferase, a nuclease, a recombinase, and a methylase.

In certain embodiments of compositions and methods of the invention, a mutagenic enzyme is a base editor attached to a DNA-binding protein that binds to one or more sites adjacent to or within the polynucleotide encoding the preselected gene of interest. In an embodiment in which the mutagenic protein is a base editor, the canvas polynucleotide sequence comprises one or more guide RNA (gRNA) target sites for the base editor. Certain embodiments of methods of the invention include expressing one or a plurality of gRNAs that are capable of directing the base editor to one or more target polynucleotide sequences present in the canvas polynucleotide sequence. As a non-limiting example, the expressed one or the plurality of gRNAs are expressed by at least one gRNA-expressing array. Art known methods of selecting and expressing gRNAs and gRNA arrays can be used in methods of the invention. Information on identification and use of nucleic-acid-guided DNA-binding proteins can be found in Anzalone, A. V. et al., Nature Biotechnology, (2020) Vol. 38:824-844 (RNA-guided DNA-binding proteins) and in Gao, F., et al., Nature Biotechnology online publication, May 2, 2016: doi:10.1038/nbt.3547 (DNA-guided DNA-binding proteins), the content of each of which is incorporated herein by reference in its entirety.

In some embodiments of the invention, the mutagenic enzyme is or is derived from a CRISPR base editor used to convert cytosines into thymines [Thuronyl, B. W. et al., Nature Biotechnology (2019) September 37(9): 1070-1079] or adenosines into guanines [see Gaudelli, N. M. et al., Nature (2017) Vol. 551, 464-4711 within sites targeted by CRISPR, a CRISPR prime editor used to introduce random indels or a template into sites targeted by CRISPR (Anzalone, A. V., et al., Nature (2019) Vol. 576, 149-157], a CRISPR nuclease and self-targeting guide RNA(s) to successively generate mutations [Perli, S. D. et al., Science (2016) September 9; 353(6304)], an integrase that successively incorporates DNA sequences into a target site [Sheth, R. U., et al., Science (2017) December 15; 358(6369)], or a polymerase attached to a mutagenic enzyme that is localized to and therefore mutates a stretch of DNA adjacent to a compatible promoter [Chen, Ht., et al, Nature Biotechnology (2020) Vol. 38, 165-168], the content of each of which is incorporated by reference herein in its entirety.)

In some embodiments the mutagenic protein is a retron. A retron is a distinct DNA sequence found in the genome of many bacteria species that codes for reverse transcriptase and a unique single-stranded DNA/RNA hybrid called multicopy single-stranded DNA (msDNA). See for example: Farzadfard, F. & T. K. Lu Science (2014) November 14; 346(6211), the content of which is incorporated herein by reference in its entirety.

In addition, certain embodiments of methods of the invention include multiplexing a mutagenic protein. In such embodiments a composition of the invention may include one or more encoded mutagenic proteins that when expressed are capable of mutagenizing a plurality of nucleic acid sequences that are contiguous with the polynucleotide sequence of the preselected gene of interest. In some embodiments, a multiplexing method of the invention can target one or more of: (i) sequence(s) 5′ of the preselected gene of interest (e.g. target multiple sites in a canvas 5′); (ii) sequence(s) 3′ of the preselected gene of interest (e.g. target multiple sites in a canvas 3′); (iii) sequence(s) 5′ and 3′ of the preselected gene of interest (e.g. target multiple sites in a canvas 5′ and 3); (iv) sequence(s) within the preselected gene of interest; and (v) sequence(s) within an intron within the preselected gene of interest, which may be described elsewhere herein.

### Introduced Detectable Mutations

A mutagenic protein is encoded in a composition of the invention, and when expressed in a method of the invention, the mutagenic protein is capable of introducing a detectable mutation into the canvas polynucleotide sequence. In some embodiments, the activity of a mutagenic protein that is encoded in a composition of the invention is capable of randomly introducing one or more detectable mutations in the canvas polynucleotide sequence. In certain embodiments, the activity of the mutagenic protein introduces the detectable mutation at one or more specific sites in the canvas polynucleotide sequence.

In some embodiments the introduced detectable mutation(s) can be positioned at 1, 2, 3, 4, 5, 6, 7, 8, or more specific sites contiguous with the polynucleotide encoding the preselected gene of interest. In certain embodiments the introduced detectable mutation is positioned 5′ of the polynucleotide that encodes the preselected gene of interest. In some embodiments the introduced detectable mutation is positioned 3′ of the polynucleotide that encodes the preselected gene of interest.

In certain embodiments, the mutagenic protein introduces one or more of the detectable mutation within the polynucleotide sequence that encodes the preselected gene of interest, in a position, or positions such that the introduction does not disrupt genotypic information of the preselected gene of interest. For example, in some embodiments, the detectable mutation is introduced into one or more of: (a) an intron in the polynucleotide encoding the preselected gene of interest, and (b) one or more synonymous bases of the polynucleotide encoding the preselected gene of interest.

A mutagenic protein included in an embodiment of the invention may capable of introducing an epigenetic change in the polynucleotide that encodes the preselected gene of interest, and the epigenetic change can be detected by sequencing, a non-limiting example of which is nanopore sequencing.

A number of introduced mutations in a canvas polynucleotide sequence may increase (also referred to herein as “accumulate”) over time. In some embodiments of methods of the invention, counting a number of introduced mutations in the canvas polynucleotide sequence at two or more time points can be used to determine a level and/or change in level of activity of the expressed product of the preselected gene of interest. As a non-limiting example, a method of the invention is performed in which the number of introduced mutations in the canvas polynucleotide sequence is determined at 1, 2, 3, 4, 5, 6, 7, or more time points and the numbers compared. An increase in the number of the introduced is proportional to the activity level of the protein product of the preselected gene of interest. As non-limiting examples, tests are performed in which the number of introduced mutations in a canvas polynucleotide sequence is determined at 5 hours, 10 hours, and 15 hours after the composition is prepared. A determination of one mutation at the five-hour time point, two mutations at the ten-hour time point, and three mutations at the fifteen-hour time point indicates a steady level of activity of the expression product of the preselected gene of interest over that time span. A count of one mutation at the five-hour time point, two mutations at the ten-hour time point, and six mutations at the fifteen-hour time point indicates an increase in the activity level of the expression product of the preselected gene of interest over that time span.

In some embodiments of methods of the invention, the accumulation of an introduced mutation in a canvas polynucleotide sequence in a composition comprising a first preselected gene of interest may be compared to the accumulation of the introduced mutation in the canvas polynucleotide sequence in a composition that includes a different preselected gene of interest instead of the first preselected gene of interest. Similarly, certain embodiments of methods of the invention, the accumulation of an introduced mutation in a canvas polynucleotide sequence in a composition comprising a first of a preselected gene of interest may be compared to the accumulation of the introduced mutation in the canvas polynucleotide sequence in a composition that includes a second of the preselected gene of interest instead of the first of the preselected gene of interest. Determined numbers and/or patterns of the introduced mutations generated in compositions that include a two or a plurality of one or more preselected genes of interest indicate activity of the expression product of the preselected gene(s) of interest, respectively, and can be compared. In addition, the sequences of a first and second of the preselected gene of interest or the preselected genes of interest can be determined using methods of the invention as described elsewhere wherein, and compared. Thus methods of the invention can be used to assess one or a plurality of preselected genes of interest and/or one or a plurality of a preselected gene of interest, thereby providing activity and sequence information for the preselected gene(s) of interest, respectively.

### Transcription/Translation, DNA Extraction and Sequencing

In certain embodiments of compositions of the invention the composition is positioned in a transcription/translation-suitable (TTS) environment and the preselected gene(s) of interest and the encoded mutagenic protein are expressed in the TTS environment. Content and use of a TTS environment are known in the art and can be used in methods and with compositions of the invention. For example: Miller, O. J., et al., Nature Methods (2006) Vol. 3, 561-570, the content of which is incorporated herein by reference in its entirety. Following the transcription/translation step, methods of the invention may include extracting DNA from the TTS environment. In some embodiments of methods of the invention, one or more conditions in the TTS environment may be adjusted in a manner suitable to induce onset, increase, decrease, cessation, of activity of a mutagenic protein that is encoded in the composition. Following transcription/translation of sequences in the composition, the resulting sequences can be assessed. A method of the invention may include a step of extracting DNA from the TTS environment during and/or after transcription/translation. It will be understood that in a TTS environment from which DNA is extracted at two or more different time points, transcription/translation may be continuing across the time points. Thus, in some embodiments of methods of the invention, DNA may be extracted during transcription/translation and in some embodiments of methods of the invention DNA may be extracted after the end of transcription/translation in the TTS.

After extraction, the extracted DNA can be assessed by one or more of (1) sequencing the preselected gene of interest and the canvas polynucleotide sequence present in the extracted DNA and (2) determining one or more characteristics of detectable mutations that were introduced into the canvas polynucleotide sequence in the extracted DNA. As indicated elsewhere herein a characteristic of the introduced mutations may be a number of the mutations and/or a pattern of the introduced mutations in the canvas polynucleotide sequence. The counted number of the detectable mutation is proportional to the activity of the sequenced preselected gene of interest, and therefore the sequencing and counting steps in the method determines the sequence and activity of the preselected gene of interest.

In some embodiments of the invention, the TTS environment is a transcription/translation (TT) reaction vessel. In some embodiments, the TTS environment is an in vitro cell, which may, but need not, be an in vitro cell in culture. In some embodiments of methods of the invention, a TTS reaction vessel comprises a plurality of compositions of the invention each comprising an independently preselected gene sequence of interest.

A non-limiting example of a method of the invention to determine a sequence and activity of a preselected gene of interest, includes extracting DNA from the TTS environment two or more times after the transcription/translation begins. The preselected gene of interest and the canvas polynucleotide in the extracted DNA from two or more different times are sequenced. The detectable mutation that were introduced into the canvas polynucleotide sequence in the extracted DNA from two or more different time are counted and/or their patterns determined. Sequences determined and the number and/or pattern of locations of the detectable mutations in at least two of the two or more DNA extractions are compared. In some embodiments, the two or more DNA extractions are separated by one or more of: at least 1 min., 5 min., 10 min., 20 min., 30 min., 40 min., 50 min., 60 min., 120 min., 180 min., 240 min., 300 min., 360 min., 420 min., 480 min., 540 min., 10 hr., 12 hr., 15 hr. 20 hr. 24 hr., 36 hr., 48 hr., 60 hr., 72 hr., 96 hr., 192 hr. 384 hr., and 800 hr.

In some embodiments of methods of the invention, the length of time between any two of the two or more DNA extractions is independently selected. As used herein the term “independently selected” means each of a given type of element may differ from others of the same type of element. So, with respect to time between DNA extractions, the time between each two consecutive DNA extractions can, but need not, be the same. As a non-limiting example, if there are three DNA extractions, the time between extractions one and two and between extractions two and three may each be five hours, or the time between extractions one and two may be 5 hours and the time between extractions two and three may be 10 hours. Thus, each independently selected length of time may be selected so as to be different than one or more other lengths of time between two DNA extractions or may be selected so as to be the same as one or more other lengths of time between two DNA extractions.

In some embodiments of methods of the invention microfluidic methods are used for one of more of the DNA extraction; counting one or more mutations introduced into a canvas polynucleotide sequence; and determining a pattern of one or more mutations introduced into a canvas polynucleotide sequence. Microfluidic methods suitable for use in methods of the invention are known in the art: See for example: Duncombe, T. A., et al., Nature Reviews Molecular Cell Biology (2015) Vol. 16, 554-567, the content of which is incorporated by reference herein in its entirety.

### Detectable Labels

An additional component that may be included in certain embodiments of compositions of the invention is a polynucleotide sequence encoding a detectable protein. In the TTs environment the encoded detectable protein is expressed and the level of the detectable protein expressed is relative to the level of the expression product of the preselected gene of interest. Thus, in some embodiments of compositions of the invention, the composition comprises a preselected gene of interest, a canvas polynucleotide sequence, a polynucleotide sequence encoding a mutagenic protein, and a polynucleotide sequence encoding a detectable protein. A non-limiting example of a detectable protein that may be included in a composition of the invention is a fluorescent or luminescent protein, although other art-known detectable proteins may also be appropriate for inclusion.

### Multiple Genes of Interest

Some methods of the invention include use of a composition of the invention to determine sequences and activities of a plurality of independently preselected genes of interest, Embodiments of such methods include (a) preparing a plurality of compositions, each comprising an independently preselected gene of interest adjacent to a canvas polynucleotide sequence and a polynucleotide sequence encoding a mutagenic protein, wherein, when expressed, activity of the encoded mutagenic protein in each composition accumulates the detectable mutation in the canvas polynucleotide sequence in the composition at a rate proportional to the molecular activity of the expression product of the independently selected preselected gene of interest in the composition; (b) positioning the plurality of the prepared compositions in a transcription/translation-suitable (TTS) environment; (c) expressing the preselected genes of interest and the encoded mutagenic proteins in the TTS environment; (d) extracting DNA from the TTS environment at a time ater the expressing; (e) sequencing the preselected genes of interest and the canvas polynucleotide sequences in the extracted DNA; and (f) counting a number of the detectable mutation in the canvas polynucleotide sequences. With this method, the counted numbers of the detectable mutations are proportional to the activity of the sequenced preselected gene of interest in each of the plurality of compositions, and the sequencing and counting identifies and determines the sequences and activities of the independently preselected genes of interest. In some embodiments, the method will also include a step of physically separating the compositions before expressing the preselected genes of interest and the encoded mutagenic proteins. In some embodiments, the physical separation occurs before extracting DNA from the TTS environment. Non-limiting means of the sequencing comprises one or more of: a high-throughput sequencing method, a Sanger sequencing method, and a barcoded high-throughput sequencing method. In certain embodiments the extracted DNA is pooled together and then sequenced, and optionally the pooled DNA is sequenced using a high-throughput sequencing method. In certain embodiments, a means for sequencing the extracted DNA includes one or more of; a nanopore sequencing methods, a PacBio sequencing method, and an Illumina sequencing method.

In some embodiments, the method described above includes physically isolating each encoding polynucleotide sequence or a plurality of identical encoding polynucleotide sequences within a cell. In certain embodiments the method of the invention includes physically isolating each encoding polynucleotide sequence or a plurality of identical encoding polynucleotide sequences within an emulsion.

Additional strategies that may be carried out using compositions and methods of the invention including in the method described above steps of (a) encoding the polynucleotide sequence(s) on phages or viruses: (b) infecting a reporter cell or plurality of reporter cells with the phages or viruses, wherein the infection comprises approximately one virus per reporter cell, wherein the reporter cell or plurality of cells each encode a recording machinery targeting a contiguous sequence in the phage or virus genome. In some embodiments, the encoded polynucleotide sequence(s) may be subjected to one or more of screening, selection, and directed evolution prior to the encoding of the polynucleotide sequence(s) on the phages or viruses. In certain embodiments, the phages or viruses encoding the polynucleotide sequence are subjected to one or more of screening, selection, and directed evolution prior to infection of the reporter cell or plurality of reporter cells. In certain embodiments, the method also includes detecting an activity of the reporter cell or plurality of reporter cells, wherein the detected activity of the reporter cell or each of the plurality of reporter cells informs an activity of all members of the evolving population. In certain embodiments, the method also includes detecting an activity of each reporter cell, wherein the detected activity of the reporter cell informs an activity of an individual member of the evolving population. A method of the invention may also include generating or identifying the plurality of independently preselected genes of interest.

In some embodiments, the plurality of independently preselected genes of interest encode a corresponding plurality of proteins, each capable of an individual activity level. Such embodiments may also include physically isolating the expressed proteins from one another and predicting activities of one or more proteins encoded by genes outside the plurality of independently preselected genes of interest based at least in part on the sequences and activities of the plurality of independently preselected genes of interest determined. A non-limiting example of a means for the predicting comprises a machine learning method, and sequences and activities determined using the method may include a training set for the machine learning method. Some embodiments of the invention also include applying the machine learning method and generating novel variants of one or more of the independently selected genes of interest.

### Cells

As described herein, a cell used in a composition and/or method of the invention may be an in vitro cell, which may or may not be a cultured cell. A non-limiting example of a type of cell that can be used in compositions and methods of the invention is a bacterial cell and an archaeal cell. In some embodiments, a cell used in a method and/or composition of the invention is a eukaryotic cell, a non-limiting example of which is: a mammalian cell, a non-human mammalian cell, an insect cell, a plant cell, and a fungal cell.

Compositions of the invention may be prepared in and/or delivered into cells of various organisms. In some aspects of the invention, a cell is a vertebrate or an invertebrate cell, in certain aspects of the invention, a cell is a eukaryotic or prokaryotic cell. A composition of the invention, in some embodiments of the invention is delivered into and/or prepared in a cell of: a bacteria, archaea, eukarya, an animal, a plant, a fungus, an insect, a fish, a reptile, an amphibian, a mammal, (horses, mice, non-human primates, humans, dogs, cats, etc.) a bird, etc.

### Sequence Variants

In a composition or method of the invention, a sequence of one or more of a preselected gene of interest, canvas polynucleotide sequence, a mutagenic protein-encoding polynucleotide sequence, and detectable label-encoding sequence may include variations, for example, one or more natural or engineered sequence changes. The terms “protein” and“polypeptide” are used interchangeably herein as are the terms “polynucleotide” and “nucleic acid” molecule. A nucleic acid molecule may comprise genetic material including, but not limited to: RNA. DNA, mRNA, cDNA, etc. As used herein with respect to polypeptides, proteins, or fragments thereof, and polynucleotides that encode such polypeptides the term “exogenous” means the one that has been introduced into a cell, cell line, organism, or organism strain and not naturally present in the wild-type background of the cell or organism strain.

In certain embodiments of the invention, a polypeptide or nucleic acid variant may be a polypeptide or nucleic acid, respectively that is modified from its “parent” polypeptide or nucleic acid sequence. Methods of the invention can be used to identify variant polynucleotide sequences and amino acid sequences and the effect, if any, of such variation on activity of the molecules.

The skilled artisan will also realize that conservative amino acid substitutions may be made in a polypeptide, for example in a Cas9 polypeptide, to design and construct a functional variant useful in a method or system of the invention. As used herein the term “variant” used in relation to polypeptides is a variant that retains a functional capability of the parent polypeptide. As used herein, a “conservative amino acid substitution” refers to an amino acid substitution that does not alter the relative charge or size characteristics of the polypeptide in which the amino acid substitution is made. Conservative substitutions of amino acids may, in some embodiments of the invention, include substitutions made amongst amino acids within the following groups: (a) M, I, L, V; (b) F, Y, W; (c) K, R, II; (d) A, G; (e) S, T; (t) Q, N; and (g) E, D. Polypeptide variants can be prepared according to methods for altering polypeptide sequence and known to one of ordinary skill in the art such. Non-limiting examples of functional variants of polypeptides for use daisy chain gene drives of the invention are functional variants of a Cas9 polypeptide, functional variants of a Cas protein, functional variants of a Cas12a protein, functional variants of reporter proteins, functional variants of a nuclease protein, etc.

As used herein the term “variant” in reference to a polynucleotide or polypeptide sequence refers to a change of 1, 2, 3, 4, 5, 6, 7, 8, 9, 10, or more nucleic acids or amino acids, respectively, in the sequence as compared to the corresponding parent sequence. For example, though not intended to be limiting, an amino acid sequence of variant reporter protein may be identical to that of its parent reporter protein sequence except that 1, 2, 3, 4, 5, 6, 7, 8, 9, 10, or more amino acid substitutions, deletions, insertions, or combinations thereof, may be present, thus making it a variant of the parent reporter protein. In another non-limiting example, the amino acid sequence of a variant Cas9 nuclease polypeptide may be identical to that of its parent Cas9 nuclease except that it has 1, 2, 3, 4, 5, 6, 7, 8, 9, 10, or more amino acid substitutions, deletions, insertions, or combinations thereof, and thus is a variant of the parent Cas9 nuclease.

Certain methods of the invention for designing and constructing methods and systems of the invention include methods to prepare and/or assess activity of variants of components of compositions of the invention. Methods provided herein, and other art-known methods can be used to prepare sequences for inclusion in compositions and methods of the invention, Methods of the invention provide means to test for activity and function of variant sequences and to determine whether an activity of a variant differs from activity of its parent molecule. Art-known methods can be used to assess relative sequence identity between two amino acid or nucleic acid sequences. For example, two sequences may be aligned for optimal comparison purposes, and the amino acid residues or nucleic acids at corresponding positions can be compared. When a position in one sequence is occupied by the same amino acid residue, or nucleic acid as the corresponding position in the other sequence, then the molecules have identity/similarity at that position. The percent identity or percent similarity between the two sequences is a function of the number of identical positions shared by the sequences (i.e., % identity or % similarity=number of identical positions/total number of positions×100). Such an alignment can be performed using any one of a number of well-known computer algorithms designed and used in the art for such a purpose. It will be understood that a variant polypeptide or polynucleotide sequence may be shorter or longer than their parent polypeptide and polynucleotide sequence, respectively. The term “identity” as used herein in reference to comparisons between sequences may also be referred to as “homology”.

### Examples

### Example 1

### Design and Testing of an Activity-Dependent Mutagenesis Assay

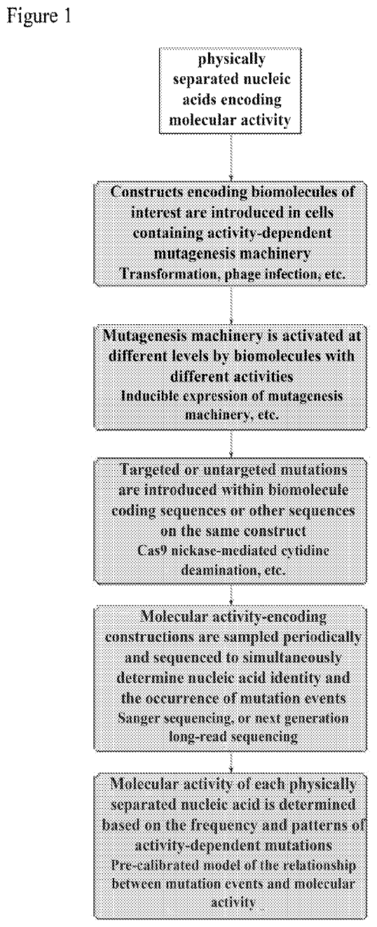

Activity-dependent mutagenesis assays directly measure the activity of an individual gene of interest. FIG. 1 shows an overview of a general work-flow for molecular activity assays of physically separated nucleic acids.

### Materials and Methods

### Genetic Circuit Construction

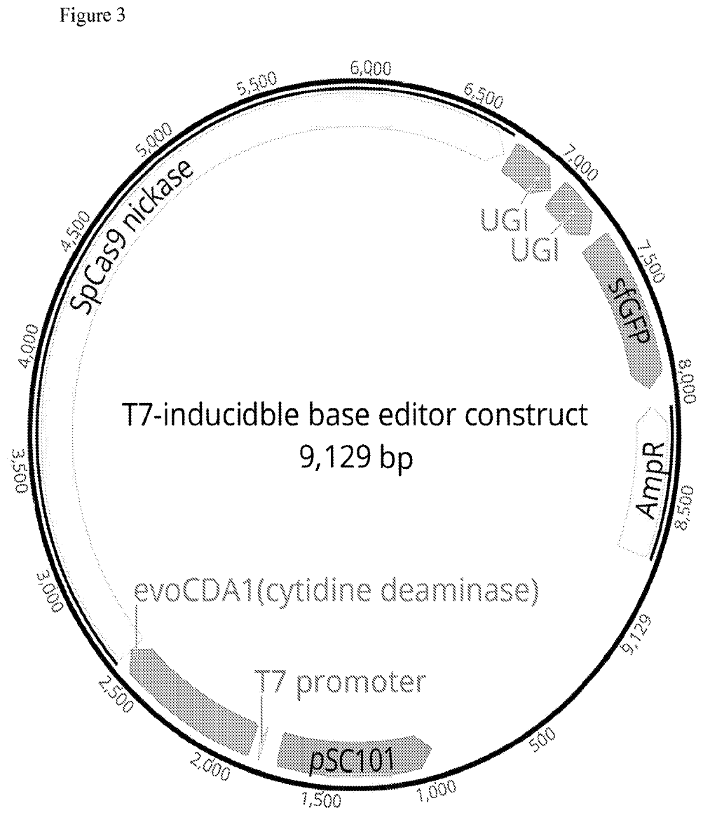

Using molecular methods known in the art, a genetic circuit was built using a sequence encoding the T7 RNA polymerase gene as the gene whose molecular activity would be measured. FIG. 3 shows a schematic diagram of a base editor map used in the study. The T7 RNA polymerase gene was placed under the transcriptional control of an IPTG (-inducible promoter (Gold Biotechnology, St. Louis, MO), and a canvas comprising the sequences of the gI, gIV, and gVI genes of M13 bacteriophage was placed downstream of the T7 RNA polymerase gene. A T7 promoter sequence was placed upstream of a sequence coding for a CRISPR/Cas9 cytosine base editor, such that the quantity of base editor protein produced was proportional to T7 RNA polymerase activity. The proportionality was confirmed by measuring the fluorescence of cell culture induced with different concentrations of IPTG, and comparing with the rates of targeted mutations in these conditions.

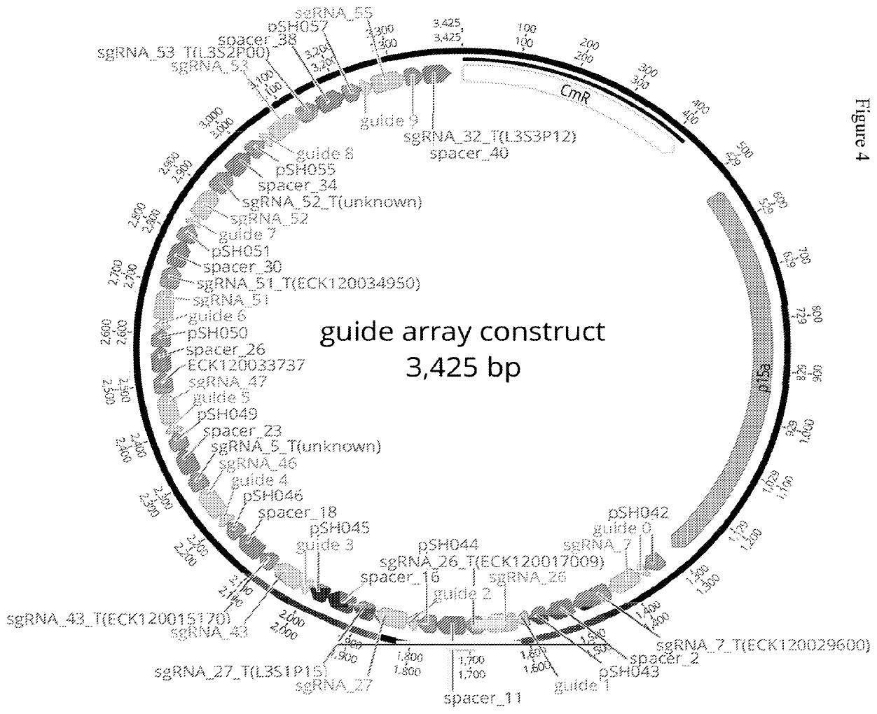

Guide RNAs targeting the CRISPR/Cas9 cytosine base editor to ten different target sequences within the canvas were expressed using one of two different guide RNA-expressing arrays. FIG. 4 provides a schematic diagram of one guide array used in the study. The two different guide arrays (“guide array 1” and “guide array 2”) expressed the same guide RNAs using different promoters and terminators (see FIG. 3). The guide arrays used in the study were on separate plasmids. SEQ ID NO; I is guide array 1 and SEQ ID NO. 2 is guide array 2 used in the study. The nucleic acid sequence of the canvas polynucleotide sequence used in the study is set forth as SEQ ID NO: 3.

The resulting constructs were transformed into E. coli bacteria using standard methods known in the art. The complete constructs were sequenced with Sanger sequencing to confirm their identities, and then simultaneously transformed into S2060 cells (wwwaddgene.org/105064/) via electroporation. The transformed cells were then plated with appropriate antibiotics to select for colonies containing the complete circuit. Chemical or electroporation transformation methods were both suitable; and in some studies electroporation was preferred for large libraries. The identity of each construct was confirmed by sequencing.

### Genetic Circuit Activity

The resulting E. coli cells containing a complete genetic circuit were grown under standard conditions. The cells were grown at 37° C. in a shaking incubator at 250 rpm and were exposed to 0, 80, or 400 μM IPTG to induce different levels of base editor activity.

### Cell Harvesting and Sequencing

E. coli containing a complete genetic circuit were grown as described above herein. At 0, 6, 24, and 40 hours, a fraction was removed. The cells in each fraction were lysed. For each fraction, the gene of interest (T7 RNA polymerase) and canvas were sequenced using a single nanopore sequencing run on an Oxford Nanopore MinION R10.3 flowcell with a different barcode for each time point. If the. The number of mutations (specifically, cytosine (C) to thymine Cr) conversions, in the canvas at each time point was counted using standard data-processing methods to analyze the data. In some studies Guppy software with CRF-based neural network model from Oxford Nanopore was used for base calling and demultiplexing. Individual targets in the canvas sequence were then identified using standard pairwise alignment algorithm and the number of C to T transitions were counted from such alignments.

### Results

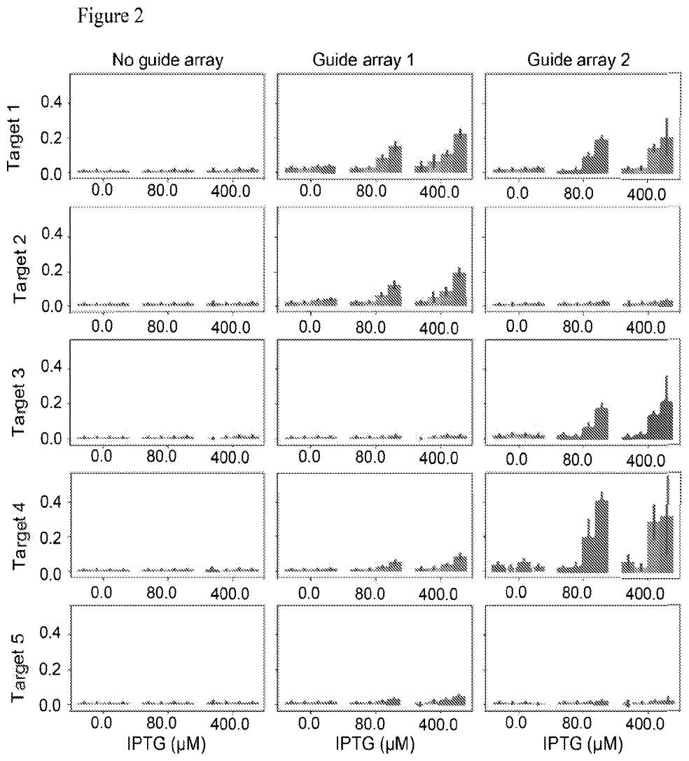

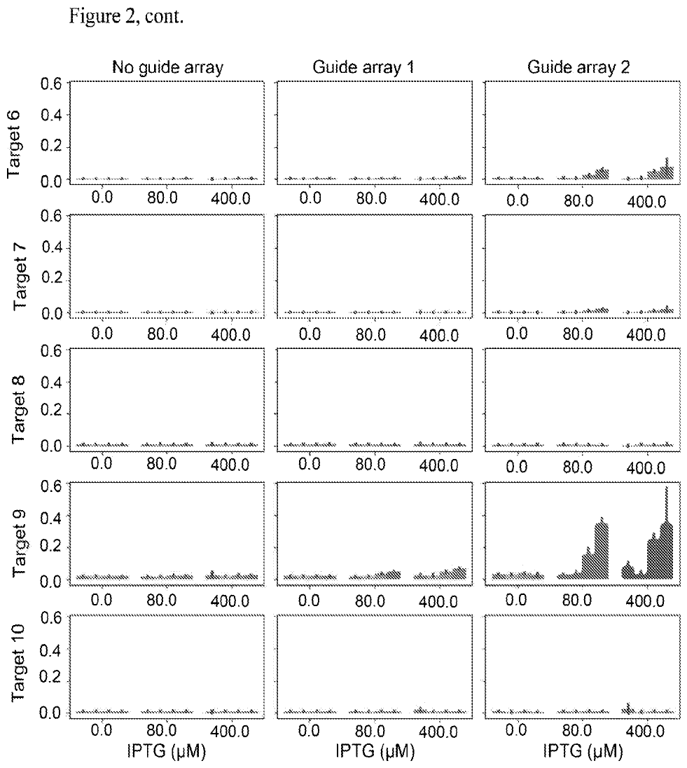

The fraction of each target site in which a cytosine had been converted to a thymine was measured (FIG. 2). The number of C to T mutations observed at target sites increased steadily with IPTG concentration and with time. Thus it was observed that higher concentrations of IPTG induced higher levels of expression, which led to a higher rate of mutation within sequences targeted by a Cas9-nickase-guided cytidine deaminase. These results demonstrated that the number of mutations in the canvas at each time point was proportional to the activity of the T7 RNA polymerase gene in the same sequencing read, and that the activity of the gene could be measured by sequencing. These results also demonstrated that signal sensitivity could be increased by growing the bacterial cells for a longer period of time.

### Example 2

### Additional Activity-Dependent Mutagenesis Assays

Activity-dependent mutagenesis assays are used to directly measure the activity of an individual gene of interest or the activity of variants of a gene of interest.

### Materials and Methods

### Genetic Circuit Construction

A genetic circuit is constructed as described above herein in Example 1, except as otherwise described herein below. Using molecular methods known in the art, a genetic circuit is built using a sequence encoding a gene of interest whose molecular activity are measured. The gene of interest is placed under the transcriptional control of an inducible promoter, and a canvas sequence within which mutagenic activity will be recorded is placed either upstream or downstream of the gene of interest. A sequence encoding a mutagenic protein is linked to the molecular activity of the gene of interest, as a non-limiting example through a promoter bound by the protein encoded by the gene of interest, such that the quantity of mutagenic protein produced is proportional to activity of the gene of interest. If the mutagenic protein can be targeted, it is targeted to mutate the canvas (as a non-limiting example, by means of concurrent expression of Cas9 guide RNAs targeting the canvas).

In some studies, a mutagenic protein that cannot be targeted is included in a composition. Thus, a dominant-negative dnaQ926 proofreading subunit of E. coli polymerase, the dam methylase of E. coli and accelerant seqA, the cytosine deaminase cda1 and repair inhibitor ugi, and the repressor emrR responsible for blocking export of mutagenic nucleobases are expressed. (Badran A. H. & D. R. Liu, Nature Communications (2015) Vol. 6, Article Number; 8425).

The resulting constructs are expressed in a transcription/translation-suitable (TTS) environment permissive for the function of the genetic circuit, for example, though not intended to be limiting, in a bacterial or mammalian cell, in an in vitro cell, or in an in vitro translational system.

### Genetic Circuit Activity

The resulting TTS environment containing a complete genetic circuit is subjected to conditions suitable to induce different levels of mutagenic protein activity.

### Harvesting and Sequencing

At various time points, for example, at 0, 6, 24, 48, and/or 96 hours, a fraction is removed and all DNA is extracted from the fraction. For each fraction, the gene of interest and canvas are sequenced using sequencing techniques including but not limited to Sanger sequencing or next-generation long-read sequencing. A different barcode may be used for each time point. The number of mutations made in the canvas at each time point is counted using standard data-processing methods to analyze the data.

### Results

Using standard data-processing methods to analyze the data, the number of mutations in the canvas at each time point are counted. The number of mutations in the canvas at each time point are proportional to the activity of the gene of interest.

A genetic circuit is designed such that it expresses a library of variants of the gene of interest, with each variant physically isolated from the other variants while still expressed in the context of the genetic circuit. Non-limiting examples of this strategy include transforming the variant library into bacteria encoding the circuit on a plasmid, or transducing mammalian cells harboring either a chromosomal or a transfected copy of the circuit with a lentivirus encoding the variant library. Sequencing analysis of the variant library and respective canvases may use a different barcode for each time point and/or for each variant. The number of mutations in the canvas at each time point are proportional to the activity of the version of the gene of interest in the same sequencing read. Different versions of certain genes of interest are observed to have higher or lower activity than that of the gene of interest.

A genetic circuit is designed such that it expresses both a mutagenic protein and a fluorescent or luminescent protein to calibrate the relative mutational activity level at each time point to light-based measurements (using light-based measurement techniques known in the art) in the TTS environment.

### Example 3

Characterizing Evolved Variants of a Gene of Interest Arising from Phage-Assisted Continuous Evolution

A phage-assisted continuous evolution is performed using a genetic circuit linking the activity of interest to production of a protein required for phage infection, such as pIII. A bacterial reporter cell line was constructed based on NEB turbo (F′) cells with an identical genetic circuit except the phage protein was replaced by the gene encoding nCas9-evoCDA1 cytosine base editor and guide RNAs targeting the regions downstream of the preselected gene of interest in the bacteriophage M13. The reporter cell line harbors a modified F plasmid and thus susceptible to phage infection. Samples of evolving bacteriophages from the evolution experiment that encode differing variants of the preselected gene of interest were used to infect reporter cells. After incubating with phage for 1 h, cells were washed with fresh media containing appropriate antibiotics. After periods of time varying from 10 minutes to 72 hours, reporter cells were removed and frozen. Canvas fragments were directly amplified from culture samples in PCR reactions using primers with unique barcodes indicating the time and phage sample, then sequenced to determine which mutations were present in the gene of interest and its corresponding molecular activity.

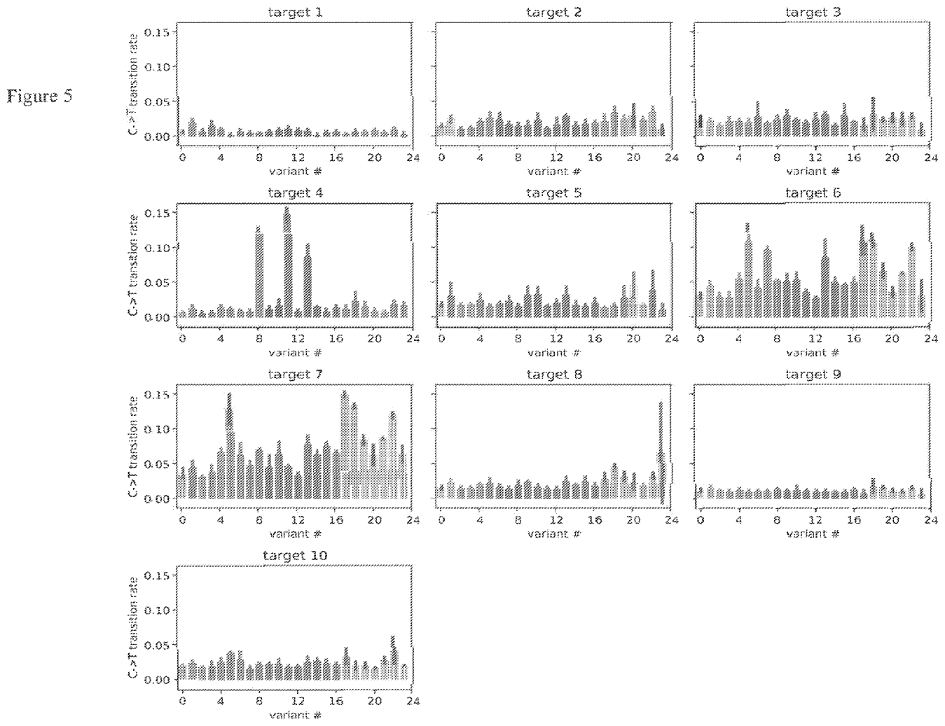

FIG. 5 provides bar graphs showing rate of targeted C to T transitions in phage variants propagated in reporter cells. Phage variants encoding T7 polymerase with various levels of activity on T7 promoter were collected from a continuous evolution experiments. Reporter cells were infected with phage variants, and samples were collected after 12 hrs of incubation. Canvas fragments were then directly amplified and barcoded for nanopore sequencing and numbers of C to T mutations were counted for each of the 10 regions targeted by the base editor using a guide array.

### Example 4

### Mammalian Cell Genotype-Phenotype Mapping

A genetic circuit is constructed that links the activity of a protein, such as a G-protein coupled receptor, to production of an nCas9-evoCDA1 cytosine base editor and a guide RNA targeting the Tet operator sequence (a related circuit for detection of GPCR activity and identification of constitutively active mutants was described in English J. G., et al., Cell, Vol. 178, Issue 3, 25 Jul. 2019, pp. 748-761. This circuit is integrated into the genome of a mammalian reporter cell line. A lentiviral library of variants of the protein with an adjacent TetO array is constructed by error-prone PCR and DNA shuffling. The lentiviral library is transduced into the reporter cell line using standard methods at low multiplicity of infection to avoid multiple insertions. After 24 to 96 hours, the cells are harvested, prepared for sequencing with barcodes indicating time point, and subjected to nanopore sequencing to determine the identity of the library member and its activity as measured by mutations in the adjacent TetO array.

### Examples 5-7

Studies were performed that included the (1) design, construction and characterization of a molecular recording system for activity reporting; (2) validation of molecular recording-based activity measurement using a library of 24 promoters; and (3) performance of high-throughput activity screening of T7 RNA polymerase variants on 13 promoter.

### Materials and Methods

### Plasmid Construction and Growth Conditions

Multilevel Golden Gate cloning and Gibson cloning were used to construct all plasmids used in the experiments. Plasmids were assembled from modules each containing a scarless transcription unit insulated by strong terminators. T4 DNA ligase and Type IIS endonucleases, BsaI, BsmBI, and PaqCI (New England Biolabs, Ipswich, MA) were used in different levels of modular assembly. To generate a library of plasmids each containing a different promoter, a lacZα cassette containing PaqCI sites was inserted between GFP and base editor RBS sequences via restriction digestion and Gibson cloning. Synthetic dsDNA fragments (Integrated DNA Technologies, Coralville, IA) each containing a different promoter and/or a bidirectional terminator, and a 24-base barcode were inserted searlessly to replace the lacZα cassette. Blue-white screening was performed as per manufacturer's instructions. NEB 10-beta cells (New England Biolabs, Ipswich, MA) were used for cloning and testing of all constructs. Transformation and selection conditions were based on manufacturer's recommendations. After plasmids were successfully cloned, all cells were grown in Davis Rich Media (see B. C. Dickinson, M. S. Packer, A. H. Badran, and D. R. Liu, Nature communications, Vol. 5, No. I, pp. 14, 2014) to lower background noise in fluorescence measurements.

### Activity Recording and Sample Collection

Commercial competent cells were transformed with plasmids carrying a canvas repeat sequence-targeting sgRNA cassette driven by strong constitutive promoter apFAB36 (see S. Kosuri, et al. Proceedings of the National Academy of Sciences, Vol. 110, No. 34, pp. 14024-14029, 2013), and rendered electrocompetent using standard procedures. Recorder plasmids were introduced either individually or as a library into the cells above via electroporation at 1700V. Cells were immediately resuspended in SOC medium and allowed to recover at 37° C. for 1 h. To eliminate plasmids that did not migrate into the cells during electroporation, cells were pelleted, washed with DNaseI reaction buffer (New England Biolabs. Ipswich, MA), resuspended in DNaseI buffer, and incubated at 37° C. for 10 min with 2 U of DNaseI. Cells were then resuspended in DRM supplemented with appropriate antibiotics at a density of approximately 0.5 OD600, and maintained at this density at 37° C. by periodically diluting the cultures with fresh DRM supplemented with appropriate antibiotics. One hundred microliters of cultures were collected hourly for GFP fluorescence measurement and PCR amplification for downstream sequencing. Samples were immediately cooled to 4-C to stop base editing activities.

### Reporter Assay and Nanopore Sequencing

Samples collected as described in the last section were washed with 10 mM HEPES buffer and loaded onto a plate reader (BMG Labtech, Ortenberg, Germany) for fluorescence measurement at 470/515 nm (Ex/Em). Absorbance at 600 nm was also measured for fluorescence normalization across different cell densities. To amplify the canvas and activity-encoding regions on the recorder plasmid construct, 0.5 μL of culture sample was directly used in a 10 μL PCR reaction using PrimeStar Max master mix (Takara Bio, San Jose, CA) under conditions recommended by the manufacturer. Primers (Azenta Life Sciences, Chelmsford, MA) used in the reactions include 24-base barcodes on the 5′ end to allow highly multiplexed sequencing across different time points on a single flow cell. PCR reactions were pooled and purified with magnetic beads (Aline Biosciences, Woburn, MA) at bead suspension to sample ratio of 0.8-1×. Sequencing library was prepared using the SQK-LSK112 Ligation Sequencing Kit and sequenced on a R10.4 flow cell as per manufacturer's instructions (Oxford Nanopore Technologies, Oxford, UK).

### Data Analysis

Basecalling and demultiplexing were performed using Guppy v5.0.7 (Oxford Nanopore Technologies, Oxford, UK) with the high-accuracy model shipped with the software. Consensus calling was performed within each group of demultiplexed reads using pbdagcon (//github.com/PacificBiosciences/pbdagcon) to identify or confirm the activity-encoding sequence. Demultiplexed raw reads were then truncated and aligned to the reference sequence of the repetitive canvas region using the Smith-Waterman algorithm implemented in Julia (see J. Bezanson, et al. SIAM Review, vol. 59, no. 1, pp. 65-98, 2017). For each read, the occurrences of mismatch where cytosine was replaced by thymidine and their positions within the canvas region were stored in a binary vector, the index of which corresponds to 7 position relative to the first base of canvas. The arithmetic sum of these vectors within each demultiplexed read group was normalized with the total number of full-length reads with the group to yield the mutation profile of the canvas sequences associated with a particular library member or variant. The area under curve (AUC) or the total number of mutations was computed to yield the metric for single-variant level activities. Samples with apparent demultiplexing or amplification problems were excluded. Data smoothing was performed by taking moving average with one neighboring data point. Time series mutation rate data were fitted to a generalized logistic function using the Levenberg-Marquardt algorithm. To validate system performance against independently measured activities, log-transformed area under curve (AUC) for each variant was plotted against corresponding log-transformed fluorescence intensity. Linear regression was performed on the log-transformed data using the least-squares algorithm.

### Validation Studies

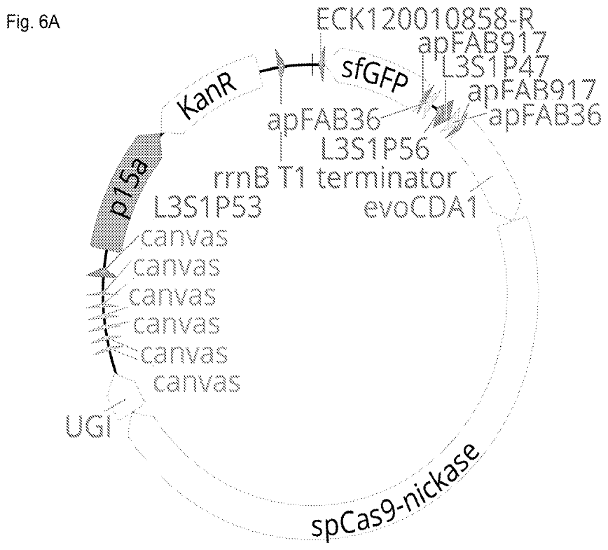

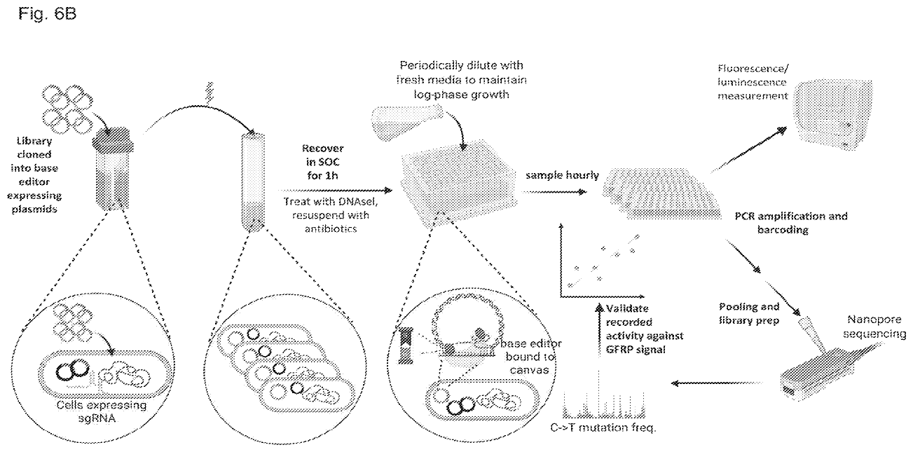

DNA fragments containing 24 promoters were synthesized and cloned into a plasmid as shown in FIG. 6A-B, such that identical promoters drove the expression of base editor and GFP. The resulting constructs were separately transformed into electrocompetent NEB 10-beta cells which already harbor a sgRNA expression plasmid. After 1 h of outgrowth, cells were treated with DNaseI and resuspended in media containing chloramphenicol and kanamycin. Samples were taken approximately every hour after outgrowth for fluorescence measurement and barcoding PCR amplification of the canvas sequences.

### Results

### (1) Results of Studies Comprising Design, Construction and Characterization of a Molecular Recording System for Activity Reporting.

Base editors are a class of molecular machinery that introduces single-base transitions to a specifically targeted DNA sequence without causing double strand breaks (N. M. Gaudelli, e t al. Nature, vol. 551, no. 7681, pp. 464-471, 2017) that have previously been used as population level molecular recorders to detect external stimuli (W. Tang and D. R. Liu, Science, vol. 360, no. 6385, p. eaap8992, 2018). Studies were performed to prepare and test molecular recorder capable of functioning at the single-cell level to measure the activity of genetically encoded signals. It was determined that as long as the concentration of guide RNA was not limiting, the expression level of a base editor within each individual cell was capable of determining the frequency of mutations in a targeted repetitive region, which was termed the “canvas”, which should accumulate over time. Modeling the relationship between base editor expression level and the measured mutation profile was used to quantify the absolute activity of any gene-encoded sequence whose activity could be coupled to production of the base editor. Sequencing samples exposed to the base editor for a brief window could differentiate between highly active and active sequences before saturation of the canvas, while sequencing samples exposed for many hours or days could differentiate between marginally and negligibly active sequences.

To test and validate a system in which the molecular activity of a functional sequence is recorded in a way such that the activity and the identity of the functional sequence can be obtained concomitantly via long-read sequencing, we designed a plasmid construct (FIG. 6A) consisting of a highly active cytosine base editor (B. W. Thuronyi, et al. et al., Nature biotechnology, vol. 37, no. 9, pp. 1070-1079, 2019), a functional sequence of interest, and a canvas, was designed and referred to as the recorder plasmid (RP). The evoCDA1 cytosine base editor was selected for use in certain embodiments of systems prepared because of its apparent distinction as the most highly active method of generating targeted mutations (J. L. Doman, et al. Nature biotechnology, vol. 38, no. 5, pp. 620-628, 2020). Its sensitivity was improved by introducing multiple target sites in the canvas. Although in principle other mutation-generating systems could also be used, the high activity and multi-site targeting of base editing is particularly suitable. For the purpose of system validation, the promoter driving the expression of the base editor was chosen as the functional sequence of interest, which was insulated from potential upstream read-through using a strong synthetic terminator. Upstream of the base editor cassette and in the reverse direction, an identical promoter was used to drive the transcription of GFP. Identical ribosome binding site (RBS) sequences were used for both coding sequences such that the activity of the promoter could be independently measured via fluorescence as a source of validation, an arrangement that proved more reliable than attempting to encode a fluorescent gene and the base editor in series (data not shown). Promoter and RBS sequences were synthetic and extensively characterized in previous works (S. Kosuri, et al. Proc. Natl. Acad. Sci. U.S.A., vol. 110, pp. 14024-14029, August 2013). The canvas sequence comprised either 5 or 10 repeats of an identical cytosine-rich CRISPR-Cas9 target, selected from a list of optimized targets with maximum editing efficiency (G. Chuai, et al. Genome biology, vol. 19, no. 1, pp. 1-18, 2018). For high-throughput testing of a library of functional sequences, the region flanked by RBS apFAB917 was replaced with a lacZα cassette flanked by two PaqCI restriction sites to allow scarless insertion of library members.

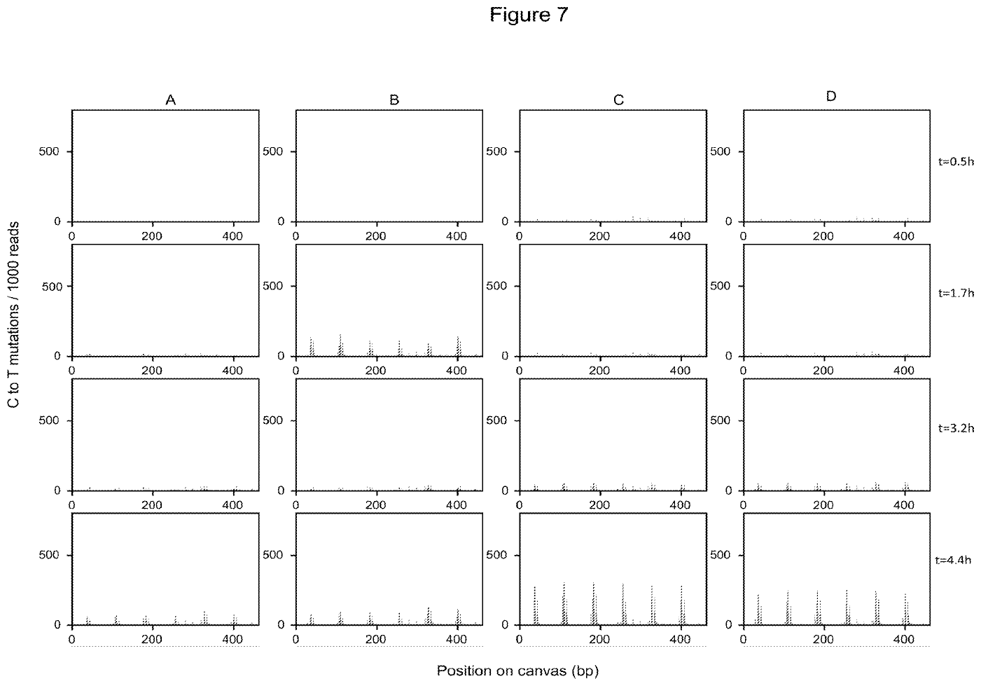

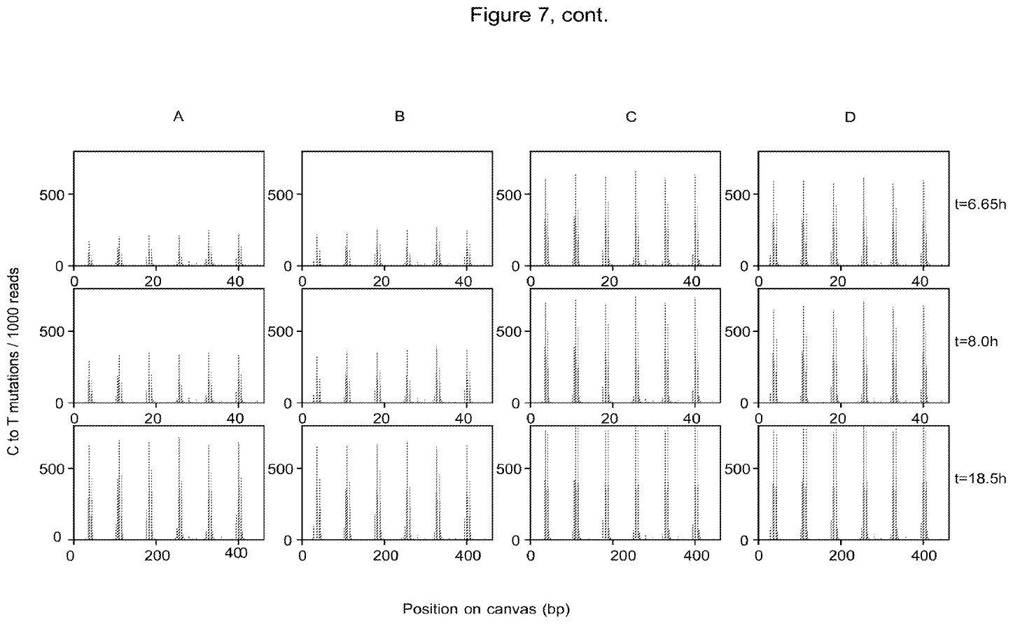

To minimize the impact of varying timing and quantity of sgRNA expression on the accumulation of mutations in the canvas, a separate plasmid was used to constitutively express sgRNA in cells that were later made electrocompetent. To probe the possible confounding effect of variation in the amount of sgRNA on mutation frequency on the canvas, cells expressing sgRNA driven by either a strong or a weak promoter were generated and introduced RPs into these cells via electroporation. After cells were allowed to recover for 1 h, liquid cultures with appropriate antibiotics were immediately started. This allowed sampling the cultures and taking snapshots of the base editing activities in the first few hours before the number of C to T mutations on some canvas reached saturation. To enable multiplexed sequencing of multiple sample collected at different time points, the region of interest was amplified with barcoded primers directly from the liquid culture and up to 480 samples were successfully sequenced on a single MinION R10.4 flow cells to yield sufficient number reads for quantification of mutations on canvas. Interestingly, the strength of the promoter driving the expression of sgRNA did not have a pronounced impact on the level of base editing activity regardless of the activity of the promoter driving the expression of the base editor, which might indicate that the sgRNA produced was in excess, and the concentration of base editor plays the predominant role in determining the number of mutations in the canvas region. As shown in FIG. 7, C to T mutations accumulated rapidly in the first 8 hours after electroporation and at a higher rate in cells where a strong promoter drove the expression of the base editor and the GFP gene, the fluorescence intensity of which confirmed the relative strengths of promoters. The rate of increase in number of mutations slowed in the next 10 hrs as the number of cytosines available for deamination decreased. The cells were allowed to grow to stationary phase overnight, which might have slowed metabolic activities in general.

(2) Results of Studies that Validated Molecular Recording-Based Activity Measurement Using a Library of 24 Promoters.

Studies were performed that demonstrated that the molecular recording-based approach (embodiments of which are described herein) can accurately quantify activity over a wide dynamic range. For the studies, 24 synthetic promoters were selected (Table 1)), the relative strengths (S. Kosuri, et al. Proc. Natl. Acad. Sci. U.S.A., vol. 110, pp. 14024-14029, August 2013) of which were previously determined to vary up to 100-fold.

### Table 1 Provides Sequences of Various Promoters Used in Certain Studies

| | | SEQ |
|---|---|---|
| | | ID |
| PROMOTER | SEQUENCE | NO |
| pro1 | GGCGCACAGCTAACACCACGTCGTCCCTATCTGCTG | 4 |
| | CCCTAGGTCTATGAGTGGTTGCTGGATAACTTTACGG | |
| | GCATGCATAAGGCTCGGTATCTATATTCAGGGAGACA | |
| | ACAACGGTTTCCCTCTACAAATAATTTTGTTTAACTTT | |
| pro2 | GGCGCACAGCTAACACCACGTCGTCCCTATCTGCTGCC | 5 |
| | CTAGGTCTATGAGTGGTTGCTGGATAACGCGGTGGGCAT | |
| | GCATAAGGCTCGTAGGCTATATTCAGGGAGACAACAAC | |
| | GGTTTCCCTCTACAAATAATTTTGTTTAACTTT | |
| pro3 | GGCGCACAGCTAACACCACGTCGTCCCTATCTGCTGCC | 6 |
| | CTAGGTCTATGAGTGGTTGCTGGATAACTTTACGGGCA | |
| | TGCATAAGGCTCGGAGGATATATTCAGGGAGACAACAA | |
| | CGGTTTCCCTCTACAAATAATTTTGTTTAACTTT | |
| pro4 | GGCGCACAGCTAACACCACGTCGTCCCTATCTGCTGCC | 7 |
| | CTAGGTCTATGAGTGGTTGCTGGATAACTTTAGGGGCAT | |
| | GCATAAGGCTCGGATGATATATTCAGGGAGACAACAAC | |
| | GGTTTCCCTCTACAAATAATTTTGTTTAACTTT | |
| pro5 | GGCGCACAGCTAACACCACGTCGTCCCTATCTGCT | 8 |
| | GCCCTAGGTCTATGAGTGGTTGCTGGATAACTTTAC | |
| | GGGCATGCATAAGGCTCGTAGGATATATTCAGGGA | |
| | GACAACAACGGTTTCCCTCTACAAATAATTTTGTTTAACTTT | |
| pro6 | GGCGCACAGCTAACACCACGTCGTCCCTATCTG | 9 |
| | CTGCCCTAGGTCTATGAGTGGTTGCTGGATAACT | |
| | TTACGGGCATGCATAAGGCTCGTAAAATATATTC | |
| | AGGGAGACAACAACGGTTTCCCTCTACAAATAATT | |
| | TTGTTTAACTTT | |
| proA | GGCGCACAGCTAACACCACGTCGTCCCTATCTGCTGCCCT | 10 |
| | AGGTCTATGAGTGGTTGCTGGATAACTTTACGGGCATGCA | |
| | TAAGGCTCGTAGGCTATATTCAGGGAGACAACAACGGTT | |
| | TCCCTCTACAAATAATTTTGTTTAACTTT | |
| proB | GGCGCACAGCTAACACCACGTCGTCCCTATCTGCT | 11 |
| | GCCCTAGGTCTATGAGTGCTTGCTGGATAACTTTAC | |
| | GGGCATGCATAAGGCTCGTAATATATATTCAGGGA | |
| | GACAACAACGGTTTCCCTCTACAAATAATTTTGTTTAACTTT | |
| apFAB49 | GGCGCGCCAAAAAGAGTATTGACTTTTATCCCTTG | 12 |
| | CGGCGAATACTTACAGCCATGTAGA | |
| apFAB52 | GGCGCGCCAAAAAGAGTATTGACTTTTATCCCTTGC | 13 |
| | GGCGACATAATTATTTCATAGTTC | |
| apFAB62 | GGCGCGCCTTGACAATTAATCATCCGGCTCGCATAATTAT | 14 |
| | TTCATTTCAG | |
| apFAB70 | GGCGCGCCTTGACATCGCATCTTTTTGTACCTATAATGTGT | 15 |
| | GGATAGAGT | |
| apFAB95 | GGCGCGCCAAAAAATTTATTTGCTTTCGCATCTTTTTGTACC | 16 |
| | TATAATGTGTGGATAATAA | |
| apFAB104 | GGCGCGCCTCGACATAAAGTCTAACCTATAGGATACTTACA | 17 |
| | GCCATAGCTT | |
| apFAB109 | GGCGCGCCTCGACAATTAATCATCCGGCTCGATACTTACAG | 18 |
| | CCATCGATT | |
| apFAB125 | GGCGCGCCTCGACATTTATCCCTTGCGGCGATATAATGTGT | 19 |
| | GGATAATCC | |
| apFAB140 | GGCGCGCCCACGGTGTTAGACATCAGGAAAATTTTTCTGTA | 20 |
| | TAATGTGTGGATGCTTA | |
| apFAB306 | GGCGCGCCTTGACAATTAATCATCCGGCTCGTAGTGTTTGTG | 21 |
| | GATGTTG | |
| apFAB311 | GGCGCGCCTTGACAATTAATCATCCGGCTCGTAGGTTGTGTG | 22 |
| | GACGGCT | |
| apFAB322 | GGCGCGCCTTGCGTATTAATCATCCGGCTCGTATAATGTGTG | 23 |
| | GATGATC | |
| apFAB345 | GGCGCGCCTTGACAATTAATCATCCGGCTCGTAGAGTATGTG | 24 |
| | GAGTATC | |
| BBa J23104 | GGCGCGCCTTGACAGCTAGCTCAGTCCTAGGTATTGTGCTA | 25 |
| | GCTTACG | |
| BBa J23116 | GGCGCGCCTTGACAGCTAGCTCAGTCCTAGGGACTATGCTA | 26 |
| | GCAGGAT | |
| pT7A1 | GGCGCGCCTCAAAAAGAGTATTGACTTAAAGTCTAACCTAT | 27 |
| | AGGATACTTACAGCCATCGAGAGCTGGG | |

An 1000-fold dynamic range in protein expression could be, achieved in combination with RBS with varying activities. To minimize the confounding effects of adjacent sequences, eight (8) insulated promoters were included among the 24 promoters chosen for system validation (J. H. Davis, et al. Nucleic acids research, vol. 39, no. 3, pp. 1131-1141, 2011). For the purpose of system validation, each promoter was separately cloned into a backbone comprising GFP, base editor, and canvas (FIGS. 6A and 8A-C), and each of the resulting constructs was individually transformed into cells expressing sgRNA driven by a strong constitutive promoter via electroporation. Because strong base editing activity can lead to saturation of the canvas in overnight cultures (data not shown), it was determined that bypassing the conventional post-transformation colony selection process and establishing liquid cultures immediately after post-electroporation outgrowth allowed capture of the base editing events at early time points. To prevent the loss of plasmids, antibiotics were added directly to liquid cultures after one hour of outgrowth. Examples of an antibiotic used are carbenicillin, kanamycin, chloramphenicol, spectinomycin. It will be understood that appropriate antibiotics for inclusion in a study were selected based on standard microbiology practice. Such cultures were maintained at constant density under log-phase growth condition using an automated liquid handler and sampled hourly for GFP assay and nanopore sequencing.

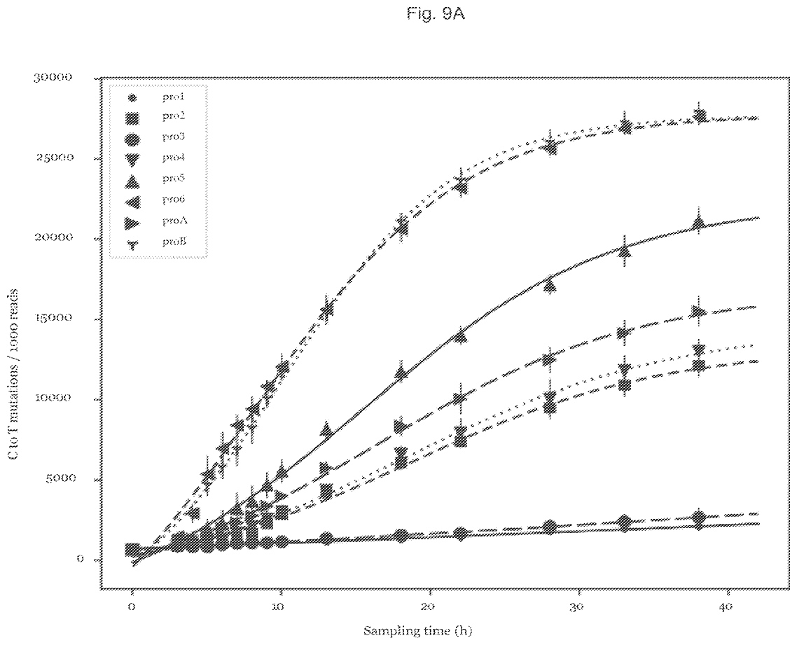

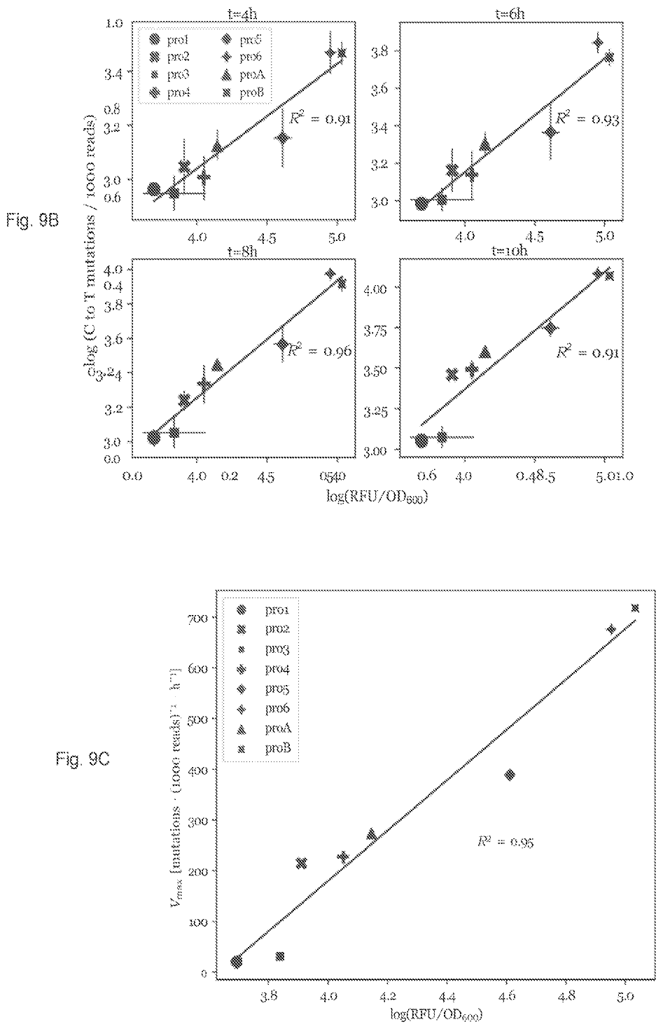

The relative strengths of promoters measured by GFP fluorescence intensity were largely consistent with those described in previous reports (data not shown). During a 42-hour period of incubation after electroporation, an accumulation of C to T transitions was observed in the canvas region over time among the majority of samples, with faster accumulation in samples with stronger promoters driving expression of the base editor. Among all the samples collected at different time points, the normalized C to T mutation frequency exhibited a wide numerical range from 500 to over 50000 mutations/10(K reads, and the mutation frequency appeared to be an increasing function of promoter activity represented by GFP fluorescence intensity (Table 2). Data smoothing of the time series was performed by taking the moving average with one neighboring data point. These time series of normalized mutation frequencies were then fitted to a generalized logistic model to assess the kinetics of base editor-mediated activity recording. Linear regression showed a strong correlation between the maximum rate of mutagenesis and the log-transformed GFP intensity (R2=0.96) among insulated promoters (FIG. 9A)). These eight promoters (FIG. 9B) also produced strong correlation between log-transformed mutation frequency and log-transformed GFP fluorescence intensity (R2>0.9) at several different time points.

Table 2 shows results demonstrating accumulation of C to T mutations in the canvas region of each construct with a different promoter driving expression of base editor. Canvas regions were amplified using barcoded primers and sequenced on a MinION R10.4 flow cell. Demultiplexed reads were aligned to the reference canvas sequence and numbers of C to T mismatches were counted and normalized against total number of full-length reads for each sample. Variable volumes of culture were replaced with fresh media every 20-30 min on an automated liquid handling platform to maintain log-phase growth.

TABLE 2
Promoter, time point, and the number of mutations per 100 reads.

| | | Number of | | | Number of |
|---|---|---|---|---|---|
| | Time | Mutations per | | Time | Mutations per |
| Promoter | Point | 1000 Reads | Promoter | Point | 1000 Reads |
| J23104 | 2 | 6031 | apFAB70 | 10 | 31624.47 |
| J23104 | 3 | 7543.65 | apFAB70 | 13 | 32213.75 |
| J23104 | 4 | 11389.47 | apFAB70 | 18 | 32842.39 |
| J23104 | 5 | 15694.71 | apFAB70 | 22 | 32954.96 |
| J23104 | 6 | 19525.35 | apFAB70 | 28 | 33547.12 |
| J23104 | 7 | 22062.04 | apFAB70 | 33 | 34627.9 |
| J23104 | 8 | 23540.06 | apFAB70 | 38 | 35308.11 |
| J23104 | 9 | 26027.69 | apFAB95 | 2 | 5576.44 |
| J23104 | 10 | 27887.56 | apFAB95 | 3 | 10320.58 |
| J23104 | 13 | 30019.6 | apFAB95 | 4 | 14894.72 |
| J23104 | 18 | 32349.12 | apFAB95 | 5 | 19331.8 |
| J23104 | 22 | 31983.5 | apFAB95 | 6 | 22448.98 |
| J23104 | 28 | 32302.58 | apFAB95 | 7 | 27134.72 |
| J23104 | 33 | 32587.74 | apFAB95 | 8 | 28133.66 |
| J23104 | 38 | 33353.49 | apFAB95 | 9 | 30051.68 |
| J23116 | 2 | 894 | apFAB95 | 10 | 30563.22 |
| J23116 | 3 | 958.79 | apFAB95 | 13 | 32760.54 |
| 123116 | 4 | 1037.67 | apFAB95 | 18 | 34892.87 |
| J23116 | 5 | 1135.55 | apFAB95 | 22 | 33990.86 |
| J23116 | 6 | 1417.98 | apFAB95 | 28 | 33792.98 |
| J23116 | 7 | 1885.4 | apFAB95 | 33 | 34098.66 |
| J23116 | 8 | 2489.13 | apFAB95 | 38 | 34380.74 |
| J23116 | 9 | 3015.63 | pT7A1 | 2 | 3060.84 |
| J23116 | 10 | 3460.14 | pT7A1 | 3 | 5032.34 |
| J23116 | 13 | 4948.92 | pT7A1 | 4 | 8858.44 |
| J23116 | 18 | 7048.99 | pT7A1 | 5 | 13487.89 |
| J23116 | 22 | 9028.33 | pT7A1 | 6 | 16610.61 |
| J23116 | 28 | 11232.34 | pT7A1 | 7 | 18276.34 |
| J23116 | 33 | 12144.97 | pT7A1 | 8 | 22306.79 |
| J23116 | 38 | 13575.12 | pT7A1 | 9 | 27879.74 |
| apFAB104 | 2 | 2852.64 | pT7A1 | 10 | 29397.95 |
| apFAB104 | 3 | 3398.11 | pT7A1 | 13 | 30937.19 |
| apFAB104 | 4 | 4501.34 | pT7A1 | 18 | 33940.05 |
| apFAB104 | 5 | 5862.31 | pT7A1 | 22 | 34748.46 |
| apFAB104 | 6 | 7125.95 | pT7A1 | 28 | 35793.99 |
| apFAB104 | 7 | 8795.58 | pT7A1 | 33 | 36085.48 |
| apFAB104 | 8 | 10297.87 | pT7A1 | 38 | 36979.09 |
| apFAB104 | 9 | 11742.49 | pro1 | 2 | 855.85 |
| apFAB104 | 10 | 13162.48 | pro1 | 3 | 890.43 |
| apFAB104 | 13 | 17413.95 | pro1 | 4 | 919.61 |
| apFAB104 | 18 | 20875.67 | pro1 | 5 | 947.2 |
| apFAB104 | 22 | 23250.96 | pro1 | 6 | 966.48 |
| apFAB104 | 28 | 26279.31 | pro1 | 7 | 988.17 |
| apFAB104 | 33 | 27041.98 | pro1 | 8 | 1051.48 |
| apFAB104 | 38 | 27628.81 | pro1 | 9 | 1101.02 |
| apFAB109 | 2 | 1134.69 | pro1 | 10 | 1125.49 |
| apFAB109 | 3 | 1158.66 | pro1 | 13 | 1198.23 |
| apFAB109 | 4 | 1155.81 | pro1 | 18 | 1356.43 |
| apFAB109 | 5 | 1148.26 | pro1 | 22 | 1502.97 |
| apFAB109 | 6 | 1157.91 | pro1 | 28 | 1828.01 |
| apFAB109 | 7 | 1171.17 | pro1 | 33 | 1986.36 |
| apFAB109 | 8 | 1187.85 | pro1 | 38 | 2135.52 |
| apFAB109 | 9 | 1211.29 | pro2 | 2 | 912.16 |
| apFAB109 | 10 | 1230.61 | pro2 | 3 | 939.57 |
| apFAB109 | 13 | 1251.89 | pro2 | 4 | 1113.72 |
| apFAB109 | 18 | 1265.86 | pro2 | 5 | 1368.17 |
| apFAB109 | 22 | 1283.83 | pro2 | 6 | 1451.71 |
| apFAB109 | 28 | 1344.38 | pro2 | 7 | 1455.99 |
| apFAB109 | 33 | 1432.22 | pro2 | 8 | 1739.45 |
| apFAB109 | 38 | 1534.66 | pro2 | 9 | 2342.12 |
| apFAB125 | 2 | 5395.02 | pro2 | 10 | 2877.32 |
| apFAB125 | 3 | 9018.17 | pro2 | 13 | 4231.97 |
| apFAB125 | 4 | 14133.94 | pro2 | 18 | 6114.01 |
| apFAB125 | 5 | 19100.65 | pro2 | 22 | 7428.7 |
| apFAB125 | 6 | 23084.62 | pro2 | 28 | 9523.32 |
| apFAB125 | 7 | 27026.75 | pro2 | 33 | 10939.31 |
| apFAB125 | 8 | 29293.18 | pro2 | 38 | 12148.05 |
| apFAB125 | 9 | 31726.79 | pro3 | 2 | 921.91 |
| apFAB125 | 10 | 32291.51 | pro3 | 3 | 938.42 |
| apFAB125 | 13 | 33708.7 | pro3 | 4 | 888.43 |
| apFAB125 | 18 | 33914.71 | pro3 | 5 | 880.26 |
| apFAB125 | 22 | 33711 | pro3 | 6 | 1011.16 |
| apFAB125 | 28 | 34508.94 | pro3 | 7 | 1097.52 |
| apFAB125 | 33 | 35491.22 | pro3 | 8 | 1124.28 |
| apFAB125 | 38 | 36372.07 | pro3 | 9 | 1141.37 |
| apFAB140 | 2 | 3273.47 | pro3 | 10 | 1185.42 |
| apFAB140 | 3 | 4514.98 | pro3 | 13 | 1398.46 |
| apFAB140 | 4 | 6947.79 | pro3 | 18 | 1605.72 |
| apFAB140 | 5 | 12402.04 | pro3 | 22 | 1715.07 |
| apFAB140 | 6 | 15853.52 | pro3 | 28 | 2146.77 |
| apFAB140 | 7 | 17451.83 | pro3 | 33 | 2449.3 |
| apFAB140 | 8 | 20627.93 | pro3 | 38 | 2697.59 |
| apFAB140 | 9 | 25041 | pro4 | 2 | 868.13 |
| apFAB140 | 10 | 26814.99 | pro4 | 3 | 901.6 |
| apFAB140 | 13 | 31274.24 | pro4 | 4 | 1012.79 |
| apFAB140 | 18 | 34060.01 | pro4 | 5 | 1141.71 |
| apFAB140 | 22 | 35000.71 | pro4 | 6 | 1376.7 |
| apFAB140 | 28 | 35261.62 | pro4 | 7 | 1738.76 |
| apFAB140 | 33 | 35814.99 | pro4 | 8 | 2148.07 |
| apFAB140 | 38 | 36055.86 | pro4 | 9 | 2623.04 |
| apFAB306 | 2 | 2597.42 | pro4 | 10 | 3075.33 |
| apFAB306 | 3 | 5459.06 | pro4 | 13 | 4385.64 |
| apFAB306 | 4 | 8514.56 | pro4 | 18 | 6655.1 |
| apFAB306 | 5 | 10456.1 | pro4 | 22 | 7975.48 |
| apFAB306 | 6 | 11949.86 | pro4 | 28 | 10093.39 |
| apFAB306 | 7 | 13607.31 | pro4 | 33 | 11842.8 |
| apFAB306 | 8 | 20470.49 | pro4 | 38 | 13045.9 |
| apFAB306 | 9 | 26396.9 | pro5 | 2 | 940.23 |
| apFAB306 | 10 | 28497.52 | pro5 | 3 | 1001.12 |
| apFAB306 | 13 | 31212.83 | pro5 | 4 | 1426.28 |
| apFAB306 | 18 | 32781.87 | pro5 | 5 | 1992.8 |
| apFAB306 | 22 | 33131.65 | pro5 | 6 | 2316.94 |
| apFAB306 | 28 | 33538.34 | pro5 | 7 | 3265.71 |
| apFAB306 | 33 | 33712.77 | pro5 | 8 | 3693.45 |
| apFAB306 | 38 | 34215.09 | pro5 | 9 | 4732.25 |
| apFAB311 | 2 | 1489.19 | pro5 | 10 | 5575.02 |
| apFAB311 | 3 | 1581.5 | pro5 | 13 | 8177.74 |
| apFAB311 | 4 | 1796.87 | pro5 | 18 | 11758.38 |
| apFAB311 | 5 | 2185.46 | pro5 | 22 | 14012.2 |
| apFAB311 | 6 | 2595.43 | pro5 | 28 | 17157.93 |
| apFAB311 | 7 | 3017.65 | pro5 | 33 | 19294.41 |
| apFAB311 | 8 | 3527.57 | pro5 | 38 | 21135.61 |
| apFAB311 | 9 | 4144.69 | pro6 | 2 | 1320.03 |
| apFAB311 | 10 | 4774.42 | pro6 | 3 | 1520.57 |
| apFAB311 | 13 | 7155.28 | pro6 | 4 | 2950.89 |
| apFAB311 | 18 | 11724.85 | pro6 | 5 | 5394.34 |
| apFAB311 | 22 | 16202.92 | pro6 | 6 | 6991.22 |
| apFAB311 | 28 | 21024.94 | pro6 | 7 | 8430.42 |
| apFAB311 | 33 | 23008.45 | pro6 | 8 | 9448.7 |
| apFAB311 | 38 | 24395.02 | pro6 | 9 | 10848.76 |
| apFAB322 | 2 | 8325.71 | pro6 | 10 | 12078.53 |
| apFAB322 | 3 | 10604.08 | pro6 | 13 | 15633.87 |
| apFAB322 | 4 | 12882.44 | pro6 | 18 | 20593.34 |
| apFAB322 | 5 | 15703.76 | pro6 | 22 | 23171.5 |
| apFAB322 | 6 | 18405.05 | pro6 | 28 | 25615.02 |
| apFAB322 | 7 | 21104.57 | pro6 | 33 | 26910.92 |
| apFAB322 | 8 | 24771.86 | pro6 | 38 | 27653.57 |
| apFAB322 | 9 | 29219.31 | proA | 2 | 943.38 |
| apFAB322 | 10 | 31153.19 | proA | 3 | 1045.04 |
| apFAB322 | 13 | 31237.88 | proA | 4 | 1341.82 |
| apFAB322 | 18 | 33097 | proA | 5 | 1674.13 |
| apFAB322 | 22 | 34202.89 | proA | 6 | 2027.13 |
| apFAB322 | 28 | 35535.63 | proA | 7 | 2376.13 |
| apFAB322 | 33 | 36298.09 | proA | 8 | 2820.35 |
| apFAB322 | 38 | 37195.8 | proA | 9 | 3409.89 |
| apFAB345 | 2 | 4678.03 | proA | 10 | 4037.41 |
| apFAB345 | 3 | 6470.82 | proA | 13 | 5775.76 |
| apFAB345 | 4 | 9411.19 | proA | 18 | 8392.53 |
| apFAB345 | 5 | 12861.98 | proA | 22 | 10073.64 |
| apFAB345 | 6 | 16868.27 | proA | 28 | 12497.55 |
| apFAB345 | 7 | 20316.86 | proA | 33 | 14162.97 |
| apFAB345 | 8 | 23747.92 | proA | 38 | 15526.67 |
| apFAB345 | 9 | 26916.18 | proB | 2 | 1186.81 |
| apFAB345 | 10 | 27946.47 | proB | 3 | 1492.76 |
| apFAB345 | 13 | 29792.69 | proB | 4 | 2939.71 |
| apFAB345 | 18 | 31821.41 | proB | 5 | 4710.71 |
| apFAB345 | 22 | 31763.63 | proB | 6 | 5820.01 |
| apFAB345 | 28 | 31741.99 | proB | 7 | 6921.77 |
| apFAB345 | 33 | 32893.58 | proB | 8 | 8256.2 |
| apFAB345 | 38 | 34148.29 | proB | 9 | 10069.04 |
| apFAB49 | 2 | 2701.51 | proB | 10 | 11697.11 |
| apFAB49 | 3 | 4182.54 | proB | 13 | 15553.38 |
| apFAB49 | 4 | 8304.14 | proB | 18 | 21071.39 |
| apFAB49 | 5 | 13257.84 | proB | 22 | 23638.17 |
| apFAB49 | 6 | 15037.16 | proB | 28 | 26007.43 |
| apFAB49 | 7 | 17227.22 | proB | 33 | 27242.71 |
| apFAB49 | 8 | 20764.63 | proB | 38 | 27877.9 |
| apFAB49 | 9 | 23455.85 | | | |
| apFAB49 | 10 | 23913.66 | | | |
| apFAB49 | 13 | 25642.23 | | | |
| apFAB49 | 18 | 28298.86 | | | |
| apFAB49 | 22 | 29184.47 | | | |
| apFAB49 | 28 | 30184.08 | | | |
| apFAB49 | 33 | 30640.3 | | | |
| apFAB49 | 38 | 30802.2 | | | |
| apFAB52 | 2 | 1975.55 | | | |
| apFAB52 | 3 | 3658.63 | | | |
| apFAB52 | 4 | 7388.3 | | | |
| apFAB52 | 5 | 11679.62 | | | |
| apFAB52 | 6 | 14718.4 | | | |
| apFAB52 | 7 | 19189.32 | | | |
| apFAB52 | 8 | 21450.76 | | | |
| apFAB52 | 9 | 26347.4 | | | |
| apFAB52 | 10 | 27754.23 | | | |
| apFAB52 | 13 | 30486.8 | | | |
| apFAB52 | 18 | 33637.05 | | | |
| apFAB52 | 22 | 34196.44 | | | |
| apFAB52 | 28 | 35319.53 | | | |
| apFAB52 | 33 | 36128.77 | | | |
| apFAB52 | 38 | 36683.17 | | | |
| apFAB62 | 2 | 2941.36 | | | |
| apFAB62 | 3 | 5362.92 | | | |
| apFAB62 | 4 | 10029.47 | | | |
| apFAB62 | 5 | 15383.92 | | | |
| apFAB62 | 6 | 19627.89 | | | |
| apFAB62 | 7 | 24300.81 | | | |
| apFAB62 | 8 | 27172.82 | | | |
| apFAB62 | 9 | 30713.39 | | | |
| apFAB62 | 10 | 32578.17 | | | |
| apFAB62 | 13 | 34613.56 | | | |
| apFAB62 | 18 | 35404.9 | | | |
| apFAB62 | 22 | 35322.62 | | | |
| apFAB62 | 28 | 35980.28 | | | |
| apFAB62 | 33 | 36252.53 | | | |
| apFAB62 | 38 | 36642.7 | | | |
| apFAB70 | 2 | 8466.57 | | | |
| apFAB70 | 3 | 11610.15 | | | |
| apFAB70 | 4 | 16583.54 | | | |
| apFAB70 | 5 | 22309.46 | | | |
| apFAB70 | 6 | 24585.32 | | | |
| apFAB70 | 7 | 26133.76 | | | |
| apFAB70 | 8 | 28724.74 | | | |
| apFAB70 | 9 | 31311.45 | | | |

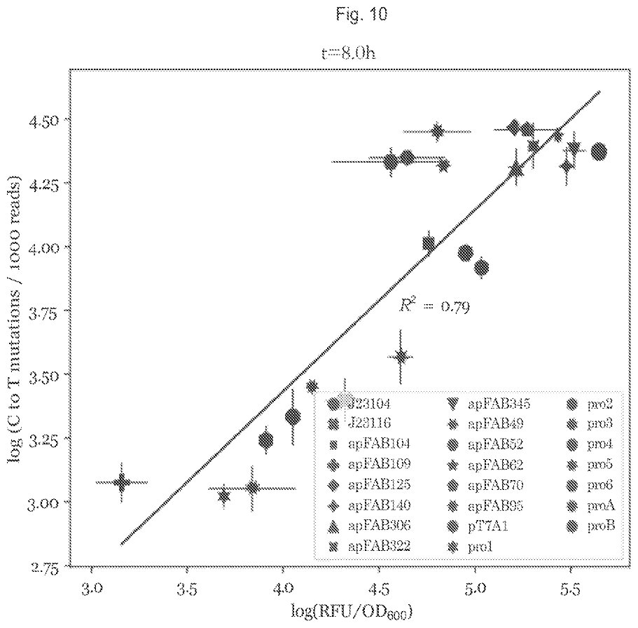

To model the numerical relationship between GFP signal and mutation frequency for all samples, the log-transformed mutation frequency observed in each promoter condition was plotted against the log-transformed normalized GFP fluorescence intensity measured at steady state in saturated cultures near the time points where Vmax was reached in most samples. As shown in FIG. 10, there was an apparent linear relationship between these two variables (R2=0.79) at 8 h after electroporation. At early time points, low-activity promoters produced indistinguishable numbers of mutations on canvas while high-activity promoters resulted mutations as apparent increasing functions of promoter strengths. At later time points, the relationship between mutation frequency and promoter strength became observable among low-activity promoters while high-activity promoters led to saturation of C to T mutations in the canvas region (See Table 2 and FIG. 10). These data indicate that the quantitative relationship between the mutation frequency in the canvas region and the underlying activity of functional sequence can be described with a tractable model over a dynamic range of at least 100-fold difference. Given the cumulative nature of base editing on canvas, a much higher dynamic range is likely to achieved if cells with low-activity functional sequences were allowed to grow in continuous culture for extended period of time until the mutation frequency reached a range of reliable quantification, a process similar to gain adjustment in a wide range of measurement techniques. Otherwise, the number of sgRNA target repeats in the canvas could be increased and baseline expression level of base editor could be tuned to increase the sensitivity of the activity recording mechanism.

(3) Results of Studies that Assessed Performance of High-Throughput Activity Screening of T7 RNA Polymerase Variants on T3 Promoter.

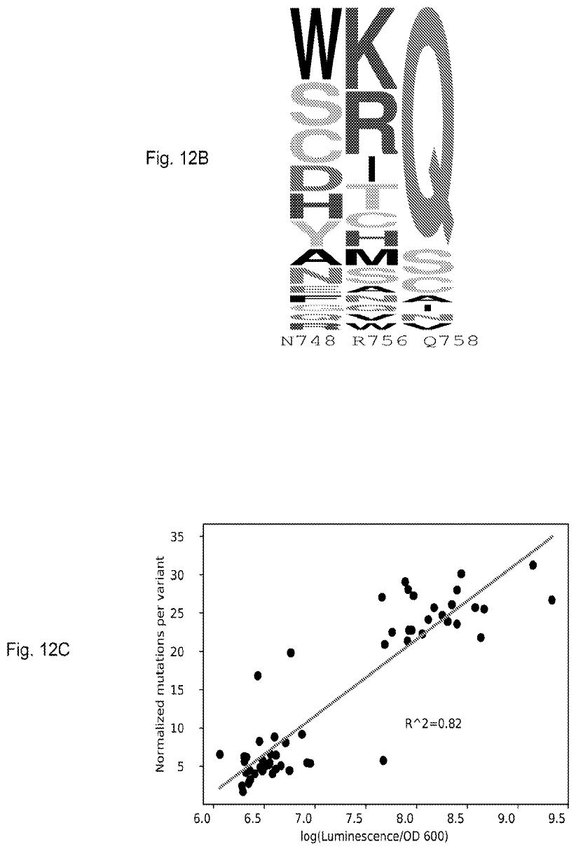

In these studies, a biological circuit was designed to couple the activities of T7 RNA polymerase variants to the number of mutations accumulated on the canvas sequence. As shown in FIG. 11A, the transcription of base editor was driven by a T3 promoter, which was recognized by a subset of variants of T7 polymerase. A weak constitutive promoter and a weak ribosomal binding site were used to prevent excessive level of expression of the 17 polymerase library, which could cause rapid saturation of the canvas and loss of activity information. This construct was introduced into cells harboring plasmids expressing the sgRNA via electroporation at the beginning of the activity recording experiment, After 12 hours of continuous growth in a turbidostat, the region of T7 polymerase library containing mutations and the canvas was amplified via PCR and subjected to long-read sequencing. Each variant in the library was assigned an activity score based on the number of C to T mutations in the canvas region normalized by the number of reads obtained for such variant. While saturated mutagenesis at the N748, R756 and Q758 yielded mostly low-activity variants (FIG. 12A) as residues at these positions are directly involved in promoter recognition, a small subset of the variants showed substantial polymerase activity as inferred from the number of C to T mutations on the canvas. Top 1% active variants are listed in Table 3.

### Table 3 Provides Positions in T7RNAP Sequence

| Position in T7 RNAP sequence | | | Normalized |
|---|---|---|---|
| 748 | 756 | 758 | Mutation |
| W | K | Q | 31.2 |
| W | S | Q | 30.1 |
| H | C | Q | 29.6 |
| W | T | Q | 29.1 |
| A | C | Q | 30.4 |
| H | R | Q | 28.0 |
| S | K | S | 27.1 |
| D | R | C | 28.0 |
| W | H | Q | 27.3 |
| D | R | V | 28.1 |
| W | K | S | 27.9 |
| D | R | I | 27.3 |
| C | T | Q | 26.7 |
| R | K | Q | 26.0 |
| C | M | Q | 25.7 |
| W | R | A | 25.1 |
| H | K | Q | 26.1 |
| C | V | Q | 25.3 |
| S | M | Q | 25.5 |
| S | S | Q | 24.1 |

As shown in FIG. 12B, the wild-type residue (Q) is highly preferred at position 758, while mutations from wild-type residues to tryptophan and lysine are most common at positions 748 and 756, respectively. To validate the activities inferred from number of C-to-T mutations accumulated in the canvas region, the base editor shown in FIG. 11A was replaced with luxAB and luciferase reporter assays were performed on variants picked either randomly or from active variants identified in FIG. 12A. All individual variants were grown to log phage and luminescence was measured after addition of 1 mM decanal and adjusted for cell growth. The linear correlation (R2=0.82) between normalized mutations per variant and log-transformed growth-adjusted luminescence indicated that the information recorded in the canvas region could be reliably used to inferred biological activities of T7 polymerase variants.

### Statement for all Examples

Means for designing constructing, integrating, and implementing such systems of the invention as well as preparing organism strains and releasing organisms of such strains, etc. that include such systems of the invention is carried out using the teaching presented herein, and in certain instances in conjunction with methods, components, and/or elements known in the art.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications cited or referred to in this application are incorporated herein in their entirety herein by reference.

What is claimed is:

1. A method of determining a sequence and activity of a preselected gene of interest, comprising:
(a) preparing a composition comprising a preselected gene of interest, a canvas polynucleotide sequence, and a polynucleotide sequence encoding a mutagenic protein, wherein the preselected gene of interest is contiguous with the canvas polynucleotide sequence and when expressed, activity of the encoded mutagenic protein accumulates a detectable mutation in the canvas polynucleotide sequence at a rate proportional to a molecular activity of the expression product of the preselected gene of interest;
(b) positioning the prepared composition in a transcription/translation-suitable (TTS) environment;
(c) expressing the preselected gene of interest and the encoded mutagenic protein in the TTS environment;
(d) extracting DNA from the TTS environment at a time after the expressing;
(e) sequencing the preselected gene of interest and the canvas polynucleotide sequence in the extracted DNA; and
(f) counting a number of the detectable mutation in the sequenced canvas polynucleotide sequence,
wherein the counted number of the detectable mutation is proportional to the activity of the sequenced preselected gene of interest, and the sequencing and counting determines the sequence and activity of the preselected gene of interest.

2. The method of claim 1, wherein the TTS environment is a transcription/translation (TT) reaction vessel, or the TTS environment is an in vitro cell, optionally wherein the in vitro cell is a cultured cell.

3-7. (canceled)

8. The method of claim 1, wherein the mutagenic protein comprises an enzyme.

9-17. (canceled)

18. The method of claim 8, wherein the enzyme is a base editor attached to a DNA-binding protein that binds to one or more sites adjacent to or within the polynucleotide encoding the preselected gene of interest, optionally wherein the canvas polynucleotide sequence comprises one or more guide RNA target sites for the base editor.

19. (canceled)

20. The method of claim 18, further comprising expressing one or a plurality of guide RNAs capable of directing the base editor to one or more target polynucleotide sequences, and optionally wherein the expressed one or the plurality of guide RNAs are expressed by at least one guide RNA-expressing array.

21-24. (canceled)

25. The method of claim 1, further comprising multiplexing the mutagenic protein and mutagenizing multiple nucleic acid sequences contiguous with the polynucleotide sequence of the preselected gene of interest.

26-28. (canceled)

29. The method of claim 1, wherein the canvas polynucleotide sequence comprises one or more predetermined polynucleotide sequences, and optionally wherein the one or more predetermined polynucleotide sequence comprises a repeated nucleic acid sequence.

30-33. (canceled)

34. The method of claim 1, further comprising extracting DNA from the TTS environment two or more times after the expressing;
(a) sequencing the preselected gene of interest and the canvas polynucleotide sequence in two or more DNA extractions;
(b) counting a number of the detectable mutation in the canvas polynucleotide sequence in the two or more DNA extractions; and
(c) comparing the sequence of the preselected gene of interest and the number of counted detectable mutations in at least two of the two or more DNA extractions.

35-36. (canceled)

37. The method of claim 1, wherein one or more of the extracting, sequencing, and counting methods comprises a microfluidics method.

38. The method of claim 1, wherein the composition further comprises a polynucleotide sequence encoding a detectable protein; the detectable protein is expressed in the TTS environment; and the level of detectable protein expressed is relative to the level of the expression product of the preselected gene of interest.

39-40. (canceled)

41. The method of claim 1, further comprising determining a pattern of the detectable mutation in the canvas polynucleotide sequence, wherein the determining occurs following at least one of step (d) and step (e).

42. A method of determining sequences and activities of a plurality of independently preselected genes of interest, comprising
(a) preparing a plurality of compositions, each comprising an independently preselected gene of interest adjacent to a canvas polynucleotide sequence and a polynucleotide sequence encoding a mutagenic protein, wherein, when expressed, activity of the encoded mutagenic protein in each composition accumulates the detectable mutation in the canvas polynucleotide sequence in the composition at a rate proportional to the molecular activity of the expression product of the independently selected preselected gene of interest in the composition;
(b) positioning the plurality of the prepared compositions in a transcription/translation-suitable (TTS) environment;
(c) expressing the preselected genes of interest and the encoded mutagenic proteins in the TTS environment;
(d) extracting DNA from the TTS environment at a time after the expressing;
(e) sequencing the preselected genes of interest and the canvas polynucleotide sequences in the extracted DNA; and
(f) counting a number of the detectable mutation in the canvas polynucleotide sequences, wherein the counted numbers of the detectable mutations are proportional to the activity of the sequenced preselected gene of interest in each of the plurality of compositions, and the sequencing and counting determines the sequences and activities of the independently preselected genes of interest.

43. The method of claim 42, further comprising physically separating the compositions before expressing the preselected genes of interest and the encoded mutagenic proteins, optionally wherein the physically separating is before extracting DNA from the TTS environment.

44-52. (canceled)

53. The method of claim 42, further comprising:
(a) encoding the polynucleotide sequence(s) on phages or viruses;
(b) infecting a reporter cell or plurality of reporter cells with the phages or viruses, wherein the infection comprises approximately one virus per reporter cell, wherein the reporter cell or plurality of cells each encode a recording machinery targeting a contiguous sequence in the phage or virus genome.

54-58. (canceled)

59. The method of claim 42, wherein the plurality of independently preselected genes of interest encode a corresponding plurality of proteins, each capable of an individual activity level and wherein the method further comprises
(i) physically isolating the expressed proteins from one another at a time subsequent to step (b) and prior to step (c); and
(ii) predicting activities of one or more proteins encoded by genes outside the plurality of independently preselected genes of interest based at least in part on the sequences and activities of the plurality of independently preselected genes of interest determined in steps (e)-(f).

60. (canceled)

61. The method of claim 59 wherein the predicting comprises a machine learning method, and optionally wherein the sequences and activities determined in steps (e) and (f) comprise a training set for the machine learning method.

62-64. (canceled)

65. A composition comprising:
(i) a preselected gene of interest contiguous to a canvas polynucleotide sequence and
(ii) a polynucleotide sequence encoding a mutagenic protein,
wherein, when expressed, activity of the encoded mutagenic protein accumulates a detectable mutation in the canvas polynucleotide sequence at a rate proportional to a molecular activity of the expression product of the preselected gene of interest.

66. The composition of claim 65, wherein the mutagenic protein comprises an enzyme.

67-90. (canceled)

91. A method of determining a sequence and activity of a preselected genes of interest, comprising
(a) preparing a composition of claim 65;
(b) positioning the prepared composition in a transcription/translation-suitable (TTS) environment;
(c) expressing the preselected gene of interest and the encoded mutagenic protein in the TTS environment;
(d) extracting DNA from the TTS environment at a time after the expressing;
(e) sequencing the preselected gene of interest and the canvas polynucleotide sequence in the extracted DNA; and
(f) assessing the detectable mutation in the canvas polynucleotide sequence, wherein the assessment of the detectable mutation correlates with the activity of the sequenced preselected gene of interest, and the sequencing and assessing determines the sequence and activity of the preselected gene of interest.

92-94. (canceled)

95. A method of determining sequences and activities of a plurality of independently preselected genes of interest, comprising
(a) preparing a plurality of compositions of claim 65, each composition comprising an independently preselected gene of interest adjacent to a canvas polynucleotide sequence and a polynucleotide sequence encoding a mutagenic protein, wherein, when expressed, activity of the encoded mutagenic protein in each composition accumulates the detectable mutation in the canvas polynucleotide sequence in the composition at a rate proportional to the molecular activity of the expression product of the independently selected preselected gene of interest in the composition;
(b) positioning the plurality of the prepared compositions in a transcription/translation-suitable (TTS) environment;
(c) expressing the preselected genes of interest and the encoded mutagenic proteins in the TTS environment;
(d) extracting DNA from the TIS environment at a time after the expressing;
(e) sequencing the preselected genes of interest and the canvas polynucleotide sequences in the extracted DNA; and
(f) assessing the detectable mutation in the canvas polynucleotide sequences, wherein the assessment of the detectable mutations correlates with the activity of the activity of the sequenced preselected gene of interest in each of the plurality of compositions, and the sequencing and assessing determines the sequences and activities of the independently preselected genes of interest.

96. (canceled)


SEQUENCE LISTING (30 sequences)

SEQ ID NO: 1 - DNA, 2082 bp, synthetic construct
  source 1..2082: mol_type = other DNA
ttgacagtagatcagagggttgctataatcgacagttccttctggtaactttgttgttttagatcacgaaagtgaaagttaaaataagcctagcccgttaccaactggaaacagtgacttaagaccgccggtcttgtccactaccttgcagtaatgcggtggacaggatcggcggttttcttttcttcacttctctgttggcacgaaaagggcaataagatttacggattactatcttgacactaccgagacagtgacatataataggaccacaccctgaacaaagtcagagttgtagagctagcaatagcaggttacaataaggctcgtccgttataaacatgaaaatgtgactaaaaaggccgctctgcggccttttttctttttgcggataaagttgatacccttacctgagttcttctgaaaataacggactttgacacgatgcttgctgctacctataataacatataccgaccgtgtgataaatagatttcgagctaggcatagcaagtgaaattaaggctggtccattaacaccttgaaaaagggaacaataaggcctccctttagggggggccttttttattgatgaaaagcaatccctcgtgaagtaactcaatagtgttctctggtatcgtattgacaactgctcagcgaaatactataatgactacacatgctcgtaaattaggatttttcagatttggaaacaaaacgttgaaaaaaggcaagtccgttatgaacgcgaaagcgtgcgaaaaaacccgcttcggcgggtttttttatagttacaatcagcagtcagaacttttacgaagaatagtggtcgctcaaccttttgacaggtgaacgctcagctcttataatgcctatcgaacctcccgacttgcggggatgtagatgtagaaatacaaggttacattaaggcccgtccgtaatcaacttgaagaagtgttccatcgggtccgaattttcggaccttttctccgcagtgaacgacactactatttcttacgagatacttattctggaagcaacggtttgacaaagtactactgtattagtataattgtcattataacccaacctaagccgggttttggacctagaaataggaagtcaaaataaggctggaccgacatgtaatcgaaagatttagaaaaaagcccgcacctgacagtgcgggctttttttttcgatattcacttccctcacagattcgttcagagataaaagcgttggtaacagtttgacatgcgtgatttaacattctataattgcacatacccgttcttggaatgatagttggagagcaagacattgcaagttccaataaggcgtgtccgataaaagcttgagaaagcaaagtaatacaaaacaggcccaggcggcctgttttgtctttttaatgctggaatacagataaggatagcgtcgttacaatagtcactcgtagaacttttgacataagtcgtattcaaagatataatataggtgcctcgcgttcttagaatacatctgagagccaaaaatggcaagttcagataaggccagaccgttaccagcttaaataagcgatcctaaagccccgaattttttataaattcggggcttttttactaggagattactttacagaagactcacttatttcacggaactggtgctgacaattgacagaccttatctacatggttataatctgaattcctacgatgaaaataaaaagcttcagatccagaaatggaaagttgaagtgaggcaggtccggtagcaactcgaaagagtgagaaaagaggggagcgggaaaccgctccccttttttcgtttttgattgagatttcaggactgtttaccgaaccttacactacgagcgataattgacacggatcttcgctgaacgtataatgagaaaccaacatgtaatttaggcagggaatagaaaacaaaagtttaagttattctaaggccagtccggaatcatcctaaaaaggagttattgaacacccgaaagggtgtttttttgttttagcccgtgtcttcttactggtggataataaagcaactgaacaacgattt

SEQ ID NO: 2 - DNA, 2082 bp, synthetic construct
  source 1..2082: mol_type = other DNA
ttgacagtagatcagagggttgctataatcgacagtataacccaacctaagccgggttttagatcacgaaagtgaaagttaaaataagcctagcccgttaccaactggaaacagtgacttaagaccgccggtcttgtccactaccttgcagtaatgcggtggacaggatcggcggttttcttttcttcacttctctgttggcacgaaaagggcaataagatttacggattactatcttgacactaccgagacagtgacatataataggaccttccttctggtaactttgttgttgtagagctagcaatagcaggttacaataaggctcgtccgttataaacatgaaaatgtgactaaaaaggccgctctgcggccttttttctttttgcggataaagttgatacccttacctgagttcttctgaaaataacggactttgacacgatgcttgctgctacctataataacatatacccgttcttggaatgatagatttcgagctaggcatagcaagtgaaattaaggctggtccattaacaccttgaaaaagggaacaataaggcctccctttagggggggccttttttattgatgaaaagcaatccctcgtgaagtaactcaatagtgttctctggtatcgtattgacaactgctcagcgaaatactataatgactacacaccctgaacaaagtcagattttcagatttggaaacaaaacgttgaaaaaaggcaagtccgttatgaacgcgaaagcgtgcgaaaaaacccgcttcggcgggtttttttatagttacaatcagcagtcagaacttttacgaagaatagtggtcgctcaaccttttgacaggtgaacgctcagctcttataatgcctattaccgaccgtgtgataaatagatgtagatgtagaaatacaaggttacattaaggcccgtccgtaatcaacttgaagaagtgttccatcgggtccgaattttcggaccttttctccgcagtgaacgacactactatttcttacgagatacttattctggaagcaacggtttgacaaagtactactgtattagtataattgtcatgcctcgcgttcttagaatacgttttggacctagaaataggaagtcaaaataaggctggaccgacatgtaatcgaaagatttagaaaaaagcccgcacctgacagtgcgggctttttttttcgatattcacttccctcacagattcgttcagagataaaagcgttggtaacagtttgacatgcgtgatttaacattctataattgcacacgaacctcccgacttgcggggttggagagcaagacattgcaagttccaataaggcgtgtccgataaaagcttgagaaagcaaagtaatacaaaacaggcccaggcggcctgttttgtctttttaatgctggaatacagataaggatagcgtcgttacaatagtcactcgtagaacttttgacataagtcgtattcaaagatataatataggtccaacatgtaatttaggcagatctgagagccaaaaatggcaagttcagataaggccagaccgttaccagcttaaataagcgatcctaaagccccgaattttttataaattcggggcttttttactaggagattactttacagaagactcacttatttcacggaactggtgctgacaattgacagaccttatctacatggttataatctgaattcctacgatgaaaataaaaagcttcagatccagaaatggaaagttgaagtgaggcaggtccggtagcaactcgaaagagtgagaaaagaggggagcgggaaaccgctccccttttttcgtttttgattgagatttcaggactgtttaccgaaccttacactacgagcgataattgacacggatcttcgctgaacgtataatgagaaaacatgctcgtaaattaggatggaatagaaaacaaaagtttaagttattctaaggccagtccggaatcatcctaaaaaggagttattgaacacccgaaagggtgtttttttgttttagcccgtgtcttcttactggtggataataaagcaactgaacaacgattt

SEQ ID NO: 3 - DNA, 1511 bp, synthetic construct
  source 1..1511: mol_type = other DNA
gtcgacatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccaagctt

SEQ ID NO: 4 - DNA, 148 bp, synthetic construct
  source 1..148: mol_type = other DNA
ggcgcacagctaacaccacgtcgtccctatctgctgccctaggtctatgagtggttgctggataactttacgggcatgcataaggctcggtatctatattcagggagacaacaacggtttccctctacaaataattttgtttaacttt

SEQ ID NO: 5 - DNA, 148 bp, synthetic construct
  source 1..148: mol_type = other DNA
ggcgcacagctaacaccacgtcgtccctatctgctgccctaggtctatgagtggttgctggataacgcggtgggcatgcataaggctcgtaggctatattcagggagacaacaacggtttccctctacaaataattttgtttaacttt

SEQ ID NO: 6 - DNA, 148 bp, synthetic construct
  source 1..148: mol_type = other DNA
ggcgcacagctaacaccacgtcgtccctatctgctgccctaggtctatgagtggttgctggataactttacgggcatgcataaggctcggaggatatattcagggagacaacaacggtttccctctacaaataattttgtttaacttt

SEQ ID NO: 7 - DNA, 148 bp, synthetic construct
  source 1..148: mol_type = other DNA
ggcgcacagctaacaccacgtcgtccctatctgctgccctaggtctatgagtggttgctggataactttacgggcatgcataaggctcggatgatatattcagggagacaacaacggtttccctctacaaataattttgtttaacttt

SEQ ID NO: 8 - DNA, 148 bp, synthetic construct
  source 1..148: mol_type = other DNA
ggcgcacagctaacaccacgtcgtccctatctgctgccctaggtctatgagtggttgctggataactttacgggcatgcataaggctcgtaggatatattcagggagacaacaacggtttccctctacaaataattttgtttaacttt

SEQ ID NO: 9 - DNA, 148 bp, synthetic construct
  source 1..148: mol_type = other DNA
ggcgcacagctaacaccacgtcgtccctatctgctgccctaggtctatgagtggttgctggataactttacgggcatgcataaggctcgtaaaatatattcagggagacaacaacggtttccctctacaaataattttgtttaacttt

SEQ ID NO: 10 - DNA, 148 bp, synthetic construct
  source 1..148: mol_type = other DNA
ggcgcacagctaacaccacgtcgtccctatctgctgccctaggtctatgagtggttgctggataactttacgggcatgcataaggctcgtaggctatattcagggagacaacaacggtttccctctacaaataattttgtttaacttt

SEQ ID NO: 11 - DNA, 148 bp, synthetic construct
  source 1..148: mol_type = other DNA
ggcgcacagctaacaccacgtcgtccctatctgctgccctaggtctatgagtggttgctggataactttacgggcatgcataaggctcgtaatatatattcagggagacaacaacggtttccctctacaaataattttgtttaacttt

SEQ ID NO: 12 - DNA, 60 bp, synthetic construct
  source 1..60: mol_type = other DNA
ggcgcgccaaaaagagtattgacttttatcccttgcggcgaatacttacagccatgtaga

SEQ ID NO: 13 - DNA, 60 bp, synthetic construct
  source 1..60: mol_type = other DNA
ggcgcgccaaaaagagtattgacttttatcccttgcggcgacataattatttcatagttc

SEQ ID NO: 14 - DNA, 50 bp, synthetic construct
  source 1..50: mol_type = other DNA
ggcgcgccttgacaattaatcatccggctcgcataattatttcatttcag

SEQ ID NO: 15 - DNA, 50 bp, synthetic construct
  source 1..50: mol_type = other DNA
ggcgcgccttgacatcgcatctttttgtacctataatgtgtggatagagt

SEQ ID NO: 16 - DNA, 61 bp, synthetic construct
  source 1..61: mol_type = other DNA
ggcgcgccaaaaaatttatttgctttcgcatctttttgtacctataatgtgtggataataa

SEQ ID NO: 17 - DNA, 51 bp, synthetic construct
  source 1..51: mol_type = other DNA
ggcgcgcctcgacataaagtctaacctataggatacttacagccatagctt

SEQ ID NO: 18 - DNA, 50 bp, synthetic construct
  source 1..50: mol_type = other DNA
ggcgcgcctcgacaattaatcatccggctcgatacttacagccatcgatt

SEQ ID NO: 19 - DNA, 50 bp, synthetic construct
  source 1..50: mol_type = other DNA
ggcgcgcctcgacatttatcccttgcggcgatataatgtgtggataatcc

SEQ ID NO: 20 - DNA, 58 bp, synthetic construct
  source 1..58: mol_type = other DNA
ggcgcgcccacggtgttagacatcaggaaaatttttctgtataatgtgtggatgctta

SEQ ID NO: 21 - DNA, 49 bp, synthetic construct
  source 1..49: mol_type = other DNA
ggcgcgccttgacaattaatcatccggctcgtagtgtttgtggatgttg

SEQ ID NO: 22 - DNA, 49 bp, synthetic construct
  source 1..49: mol_type = other DNA
ggcgcgccttgacaattaatcatccggctcgtaggttgtgtggacggct

SEQ ID NO: 23 - DNA, 49 bp, synthetic construct
  source 1..49: mol_type = other DNA
ggcgcgccttgcgtattaatcatccggctcgtataatgtgtggatgatc

SEQ ID NO: 24 - DNA, 49 bp, synthetic construct
  source 1..49: mol_type = other DNA
ggcgcgccttgacaattaatcatccggctcgtagagtatgtggagtatc

SEQ ID NO: 25 - DNA, 48 bp, synthetic construct
  source 1..48: mol_type = other DNA
ggcgcgccttgacagctagctcagtcctaggtattgtgctagcttacg

SEQ ID NO: 26 - DNA, 48 bp, synthetic construct
  source 1..48: mol_type = other DNA
ggcgcgccttgacagctagctcagtcctagggactatgctagcaggat

SEQ ID NO: 27 - DNA, 69 bp, synthetic construct
  source 1..69: mol_type = other DNA
ggcgcgcctcaaaaagagtattgacttaaagtctaacctataggatacttacagccatcgagagctgcg

SEQ ID NO: 28 - DNA, 9592 bp, synthetic construct
  source 1..9592: mol_type = other DNA
cgactcactatagggagagcggcgtcgtaactagtagtgtcgtaaataaaaaaggcacgtcagatgacgtgccttttttcttgtgttagtgatggtggtggtgatggcttcccttgtagagttcgtccattccgtgagtaatgcctgcagcggtcacgaattctaacagaaccatgtgatcgcgtttttcgttcggatctttgctcaggactgactgggtgctcaggtagtggttgtcaggtaacagtacaggcccgtcaccaatcggcgtgttttgttgataatgatccgctaattgaacgctgccgtcttccacgttatggcgaattttgaaatttgccttgataccgtttttctgtttatcggccgtaatatacacgttatgagaattaaagttatattccagcttatggcctaaaatgttgccgtcttctttgaagtcgatacctttcagttcgatacggtttactaatgtgtcgccttcaaatttcacttccgcacgcgttttataggtgccatcgtccttgaagctgattgtacgctcttgaacatatccttcaggcatggctgatttgaaaaaatcgtgttgtttcatatgatctgggtaacgactgaagcactgaacgccataggtcagggtggtcactaaggtgggccacggaacgggtaatttgcctgttgtgcaaataaacttcagggtcaatttaccattggtagcgtcaccttcgccttcgccgcgcacgctaaatttgtgaccgttcacgtcgccgtctaattccactaagatcggtactacacccgtaaacagctcttcacctttgctcatatgaagcaggtgtaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagcacctgcgaagatcttaatctagcggaggagactttcatatgtctaccgacgctgaatacgttcgtatccacgaaaaactggacatctacaccttcaaaaaacagttctctaacaacaaaaaatctgtttctcaccgttgctacgttctgttcgaactgaaacgtcgtggtgaacgtcgtgcttgcttctggggttacgctgttaacaaaccgcagtctggtaccgaacgtggtatccacgctgaaatcttctctatccgtaaagttgaagaatacctgcgtgacaacccgggtcagttcaccatcaactggtactcttcttggtccccgtgcgctgactgcgctgaaaaaatcctggaatggtacaaccaggaactgcgtggtaacggtcacaccctgaagatatgggtctgcaagctgtactacgaaaaaaacgctcgtaaccagatcggtctgtggaacctgcgtgacaacggtgttggtctgaacgttatggtttctgaacactaccagtgctgccgtaaaatcttcatccagtcttctcacaaccagctgaacgaaaaccgttggctggaaaaaaccctgaaacgtgctgaaaaacgtcgttctgaactgtctatcatgttccaggttaaaatcctgcacaccaccaaatctccggctgttagcggcggttcttccggtggctcctctggttctgaaaccccgggtacctctgaatctgctaccccggaatctagcggtggctcctctggcggttctgataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacagcggcggtagcggcggtagcggtggtagcactaacctgagcgacatcattgagaaggagactggtaaacagctggttattcaggagtccatcctgatgctgccggaggaggtggaggaagtgatcggcaacaagccagagtctgacatcctggtgcacaccgcctacgacgagtccaccgatgagaacgtgatgcttctgacctctgacgccccggagtataagccgtgggccctggttatccaggattctaacggcgagaacaagatcaagatgctgagcggtggttccggtggttctggtggtagcaccaacctgtctgacatcatcgagaaggagacgggcaagcagctggttattcaggagtccatcctgatgctgccggaggaggtggaggaagtgatcggcaacaagccagagtctgacatcctggtgcacaccgcctacgacgagtccaccgatgagaacgtgatgcttctgacctctgacgccccggagtataagccgtgggccctggttatccaggattccaacggtgagaacaaaatcaaaatgctgtaacctcggtaccaaattccagaaaagaggcctcccgaaaggggggccttttttcgttttggtccacgaaaaaggcgcgccattaatcatccggaatgcaccatacacatatgtctcaagtgaggtagcaacatgacaattcttgtggctggctcgaattgcccagaaccgcacccaccgaggtcacttctctgttggcacgaaaagggcaataagatttacggattactatcgcccagaaccgcacccaccgtggtgaaaagcaatccctcgtgaagtaactcaatagtgttctctggtatcgtagcccagaaccgcacccaccggggttacaatcagcagtcagaacttttacgaagaatagtggtcgctcaaccttgcccagaaccgcacccaccggggtgaacgacactactatttcttacgagatacttattctggaagcaacggtgcccagaaccgcacccaccgtggtattcacttccctcacagattcgttcagagataaaagcgttggtaacagtgcccagaaccgcacccaccgaggctggaatacagataaggatagcgtcgttacaatagtcactcgtagaactttaagatacaatggtaaccacaagaattgcaatgaccatgccacattacataacccaattattgaaggcctcccaaatcggggggccttttttattgataacaaaaacgaagacggcgcgagacccacagtgactgcatgctagcggtctctacgatacagcggccgctgtagcctgccatggaaaatcgatgttcttaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagtctgacgctctattcaacaaagccgccgtccatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 29 - DNA, 2072 bp, synthetic construct
  source 1..2072: mol_type = other DNA
tccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctaaatgtacctataaccagaccgttcagctggatattacggcctttttaaagaccgtaaagaaaaataagcacaagttttatccggcctttattcacattcttgcccgcctgatgaatgctcatccggagttccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcactatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgccatgggcatgtagtcaaaagcctccggtcggaggcttttgacttggctgaggagtgccgttaattaagtccgtggggaaaaaatcatggcaattctggaagaaatagcgctttcagccggcaaacctgaagccggatctgcgattctgataacaaactagcaacaccagaacagcccgtttgcgggcagcaaaacccgtaccctaggtctagggcggcggatttgtcctactcaggagagcgttcaccgacaaacaacagataaaacgaaaggcccagtctttcgactgagcctttcgttttatttgatgcctctagttgggcgcgccgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatcctgtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaagacccacccgtgaaggtgagccagtgagttgattgcagtccagttacgctggagtcaagactagtcgtctccacctgcatacagtgacgaccgtaataaaaaaggcacgtcagatgacgtgccttttttcttgtgttagcaccgactcggtgccactttttcaagttgataacggactagccttattttaacttgctatttctagctctaaaaccggtgggtgcggttctgggcagatcttcaatgaatctattatacgagccggatgattaatagtcaatactctttttggcgcgccctaaccaggataagcaggtgatactgagacggcggccgcggcacgtaagaggt

SEQ ID NO: 30 - DNA, 11371 bp, synthetic construct
  source 1..11371: mol_type = other DNA
tgtgaaattgttatccgctcagagggcacaatcctattccgcgctatccgacaatctccaagacattaggtggagttcagttcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagtctgacgctctattcaacaaagccgccgtccatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtgtacttcgttcagtcttgtgtcccagttaccagggttgtaaaacgacggccagtcatgcataatccgcacgcatctggaataaggaagtgccattccgcctgacctcgactcactatagggagagcggcgtcgtaactagtagtgtcgtaaataaaaaaggcacgtcagatgacgtgccttttttcttgtgttacagcattttgattttgttctcaccgttggaatcctggataaccagggcccacggcttatactccggggcgtcagaggtcagaagcatcacgttctcatcggtggactcgtcgtaggcggtgtgcaccaggatgtcagactctggcttgttgccgatcacttcctccacctcctccggcagcatcaggatggactcctgaataaccagctgcttgcccgtttccttctcgatgatgtcagacaggttggtgctaccaccagaaccaccggaaccaccgctcagcatcttgatcttgttctcgccgttagaatcctggataaccagggcccacggcttatactccggggcgtcagaggtcagaagcatcacgttctcatcggtggactcgtcgtaggcggtgtgcaccaggatgtcagactctggcttgttgccgatcacttcctccacctcctccggcagcatcaggatggactcctgaataaccagctgtttaccagtctccttctcaatgatgtcgctcaggttagtgctaccaccgctaccgccgctaccgccgctgtcacctcctagctgactcaaatcaatgcgtgtttcataaagaccagtgatggattgatggataagagtggcatctaaaacttcttttgtagacgtatatcgtttacgatcaattgttgtatcaaaatatttaaaagcagcgggagctccaagattcgtcaacgtaaataaatgaataatattttctgcttgttcacgtattggtttgtctctatgtttgttatatgcactaagaactttatctaaattggcatctgctaaaataacacgcttagaaaattcactgatttgctcaataatctcatctaaataatgcttatgctgctccacaaacaattgtttttgttcgttatcttctggactacccttcaacttttcataatgactagctaaatataaaaaattcacatatttgcttggcagagccagctcatttcctttttgtaattctccggcactagccagcatccgtttacgaccgttttctaactcaaaaagactatatttaggtagtttaatgattaagtcttttttaacttccttatatcctttagcttctaaaaagtcaatcggatttttttcaaaggaacttctttccataattgtgatccctagtaactctttaacggattttaacttcttcgatttccctttttccaccttagcaaccactaggactgaataagctaccgttggactatcaaaaccaccatatttttttggatcccagtcttttttacgagcaataagcttgtccgaatttctttttggtaaaattgactccttggagaatccgcctgtctgtacttctgttttcttgacaatattgacttggggcatggacaatactttgcgcactgtggcaaaatctcgccctttatcccagacaatttctccagtttccccattagtttcgattagagggcgtttgcgaatctctccatttgcaagtgtaatttctgttttgaagaagttcatgatattagagtaaaagaaatattttgcggttgctttgcctatttcttgctcagacttagcaatcattttacgaacatcataaactttataatcaccatagacaaactccgattcaagttttggatatttcttaatcaaagcagttccaacgacggcatttagatacgcatcatgggcatgatggtaattgttaatctcacgtactttatagaattggaaatcttttcggaagtcagaaactaatttagattttaaggtaatcactttaacctctcgaataagtttatcattttcatcgtatttagtattcatgcgactatccaaaatttgtgccacatgcttagtgatttggcgagtttcaaccaattggcgtttgataaaaccagctttatcaagttcactcaaacctccacgttcagctttcgttaaattatcaaacttacgttgagtgattaacttggcgtttagaagttgtctccaatagtttttcatctttttgactacttcttcacttggaacgttatccgatttaccacgatttttatcagaacgcgttaagaccttattgtctattgaatcgtctttaaggaaactttgtggaacaatgtgatcgacatcataatcacttaaacgattaatatctaattcttggtccacatacatgtctcttccattttggagataatagagatagagcttttcattttgcaattgagtattttcaacaggatgctctttaagaatctgacttcctaattctttgataccttcttcgattcgtttcatacgctctcgcgaatttttctggcccttttgagttgtctgattttcacgtgccatttcaataacgatattttctggcttatgccgccccattactttgaccaattcatcaacaacttttacagtctgtaaaataccttttttaatagcagggctaccagctaaatttgcaatatgttcatgtaaactatcgccttgtccagacacttgtgctttttgaatgtcttctttaaatgtcaaactatcatcatggatcagctgcataaaattgcgattggcaaaaccatctgatttcaaaaaatctaatattgttttgccagattgcttatccctaataccattaatcaattttcgagacaaacgtccccaaccagtataacggcgacgtttaagctgtttcatcaccttatcatcaaagaggtgagcatatgttttaagtctttcctcaatcatctccctatcttcaaataaggtcaatgttaaaacaatatcctctaagatatcttcattttcttcattatccaaaaaatctttatctttaataatttttagcaaatcatggtaggtacctaatgaagcattaaatctatcttcaactcctgaaatttcaacactatcaaaacattctatttttttgaaataatcttcttttaattgcttaacggttacttttcgatttgttttgaagagtaaatcaacaatggctttcttctgttcacctgaaagaaatgctggttttcgcattccttcagtaacatatttgacctttgtcaattcgttataaaccgtaaaatactcataaagcaaactatgttttggtagtactttttcatttggaagatttttatcaaagtttgtcatgcgttcaataaatgattgagctgaagcacctttatcgacaacttcttcaaaattccatggggtaattgtttcttcagacttccgagtcatccatgcaaaacgactattgccacgcgccaatggaccaacataataaggaattcgaaaagtcaagattttttcaatcttctcacgattgtcttttaaaaatggataaaagtcttcttgtcttctcaaaatagcatgcagctcacccaagtgaatttgatggggaatagagccgttgtcaaaggtccgttgcttgcgcagcaaatcttcacgatttagtttcaccaataattcctcagtaccatccattttttctaaaattggtttgataaatttataaaattcttcttggctagctcccccatcaatataacctgcatatccgttttttgattgatcaaaaaagatttctttatacttttctggaagttgttgtcgaactaaagcttttaaaagagtcaagtcttgatgatgttcatcgtagcgtttaatcattgaagctgataggggagccttagttatttcagtatttactcttaggatatctgaaagtaaaatagcatctgataaattcttagctgccaaaaacaaatcagcatattgatctccaatttgcgccaataaattatctaaatcatcatcgtaagtatcttttgaaagctgtaatttagcatcttctgccaaatcaaaatttgatttaaaattaggggtcaaacccaatgacaaagcaatgagattcccaaataagccatttttcttctcaccggggagctgagcaatgagattttctaatcgtcttgatttactcaatcgtgcagaaagaatcgctttagcatctactccacttgcgttaatagggttttcttcaaataattgattgtaggtttgtaccaactggataaatagtttgtccacatcactattatcaggatttaaatctccctcaatcaaaaaatgaccacgaaacttaatcatatgcgctaaggccaaatagattaagcgcaaatccgctttatcagtagaatctaccaatttttttcgcagatgatagatagttggatatttctcatgataagcaacttcatctactatatttccaaaaataggatgacgttcatgcttcttgtcttcttccaccaaaaaagactcttcaagtcgatgaaagaaactatcatctactttcgccatctcatttgaaaaaatctcctgtagataacaaatacgattcttccgacgtgtataccttctacgagctgtccgtttaagacgagtcgcttccgctgtctctccactgtcaaataaaagagcccctataagattttttttgatactgtggcggtctgtatttcccagaaccttgaactttttagacggaaccttatattcatcagtgatcaccgcccatccgacgctatttgtgccgatagctaagcctattgagtatttcttatcagaaccgccagaggagccaccgctagattccggggtagcagattcagaggtacccggggtttcagaaccagaggagccaccggaagaaccgccgctaacagccggagatttggtggtgtgcaggattttaacctggaacatgatagacagttcagaacgacgtttttcagcacgtttcagggttttttccagccaacggttttcgttcagctggttgtgagaagactggatgaagattttacggcagcactggtagtgttcagaaaccataacgttcagaccaacaccgttgtcacgcaggttccacagaccgatctggttacgagcgtttttttcgtagtacagcttgcagacccatatcttcagggtgtgaccgttaccacgcagttcctggttgtaccattccaggattttttcagcgcagtcagcgcacggggaccaagaagagtaccagttgatggtgaactgacccgggttgtcacgcaggtattcttcaactttacggatagagaagatttcagcgtggataccacgttcggtaccagactgcggtttgttaacagcgtaaccccagaagcaagcacgacgttcaccacgacgtttcagttcgaacagaacgtagcaacggtgagaaacagattttttgttgttagagaactgttttttgaaggtgtagatgtccagtttttcgtggatacgaacgtattcagcgtcggtagacatatgaaagtctccccggctagattaagataaagttaaacaaaattatttgtagagggaaaccgttgttgtctcccagatactgcggctgcaggtctttctccctttagtgagggttaattcgcccaggggcgcgccattaatcatccggaatgcaccatacacatatgttttgttatcaataaaaaaggcccccgatttgggaggccttttttcgaaaataacttttcgaaaaaaggcctcccaaatcgggggccttttttatagcaacaaaaacgaactcggcgcgccaaaaaatttatttgctttcgcatctttttgtacctagatttaacgtatcccgaatcttaatctagcaggggacactttcatatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgctaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcacaattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgctggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacagaagagcaagcaatggaactgctcttcaaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacacaagaaaaaaggcacgtcatctgacgtgccttttttatttacgaaaaaggcgcgccattaatcatccggaatgcaccatacacatatggcccagaaccgcacccaccgtggtactttcagacgagaaaacgaaacggcccagaaccgcacccaccgtggttccgggacgcattttaaagaagaggcccagaaccgcacccaccggggaacagaggcctagtccgatgcatgtgcccagaaccgcacccaccgtggtcgcccctagaaacgaggggtccctgcccagaaccgcacccaccgaggactattatgagcgtttaagtacattgcccagaaccgcacccaccgtggacgttaatatattatggacgcccgcgcccagaaccgcacccaccggggattgttaggtagctaaacattacgtgcccagaaccgcacccaccgaggacaaatacaattaagaaagtctcgcgcccagaaccgcacccaccggggggccagtcccaccagcgcggagtaagcccagaaccgcacccaccgtgggtaataatgattaaggtcaccaggttaacccaattattgaaggcctcccaaatcggggggccttttttattgataacaaaaacgaagacggcgcgagacccacagtgactgcatgctagcggtctctacgatacagcggccgctgtagcctgccatggaaaatcgatgttcttaggctaggtggaggctcagtgatgataagtctgcgatggtggatgcatgtgtcatggtcatagctgtttcctgtccgattctgcttctttctacctgagcaatacgtcatagctgtttcctg